(19) United States
(12) Patent Application Publication
(10) Pub. No.: US 2016/0318897 A1
(43) Pub. Date: Nov. 3, 2016

(54) AZOLE COMPOUNDS CARRYING AN IMINE-DERIVED SUBSTITUENT

(71) Applicant: BASF SE, Ludwigshafen (DE)

(72) Inventors: Karsten KOERBER, Eppelheim (DE); Wolfgang VON DEYN, Neustadt (DE); Jochen DIETZ, Karlsruhe (DE); Arun NARINE, Mannheim (DE); Jean-Yves WACH, Mannheim (DE); Raffael KOLLER, Mannheim (DE); Joachim DICKHAUT, Heidelberg (DE)

(21) Appl. No.: 15/104,063

(22) PCT Filed: Dec. 17, 2014
(86) PCT No.: PCT/EP2014/078216

§ 371 Date: Jun. 13, 2016

Related U.S. Application Data
(60) Provisional application No. 61/917,404, filed on Dec. 18, 2013.

(51) Int. Cl. C07D 401/06; A01N 43/647; A01N 43/56

(52) U.S. Cl. CPC C07D 401/06; A01N 43/56; A01N 43/647

(57) ABSTRACT

The present invention relates to azole compounds of formula I

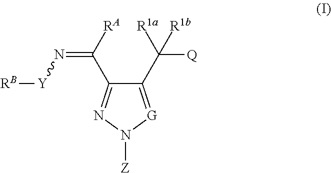

wherein the variables are as defined in the claims or the description.

The invention also relates to a method for controlling invertebrate pests by using these compounds and to plant propagation material and to an agricultural and a veterinary composition comprising said compounds.

The present invention relates to azole compounds carrying an imine-derived substituent which are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

Invertebrate pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an on-going need for new agents for combating invertebrate pests, in particular insects, arachnids and nematodes.

Related insecticidal azole compounds are described in WO 2013/063282 and WO 2013/173218. However, these documents do not describe compounds having the characteristic substituents and substituents' arrangement as claimed in the present invention.

It is an object of the present invention to provide compounds that have a good pesticidal activity, in particular insecticidal activity, and show a broad activity spectrum against a large number of different invertebrate pests, especially against difficult to control arthropod pests and/or nematodes.

It has been found that these objectives can be achieved by azole compounds of the formula I below carrying an imine-derived substituent, by their stereoisomers and by their salts, in particular their agriculturally or veterinarily acceptable salts.

Therefore, in a first aspect, the invention relates to azole compounds of formula I

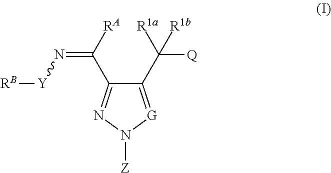

wherein
Q is a radical of formula Q-1, Q-2, Q-3, Q-4 or Q-5

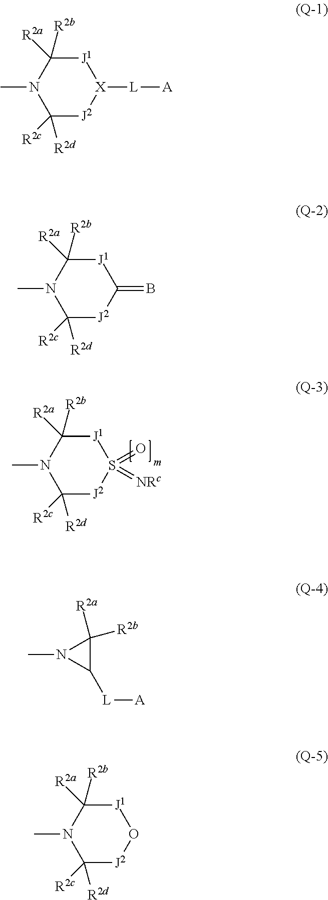

J¹ is a direct bond, —C(R^{3a}R^{3b})—, —C(R^{3a}R^{3b})—C(R^{3a}R^{3b})— or —C(═B)—;
J² is a direct bond, —C(R^{3c}R^{3d})— or —C(═B)—;
X is selected from CR^{3e} and N;
L is a single bond or is selected from the group consisting of C₁-C₁₂-alkylene, C₁-C₁₂-haloalkylene, C₂-C₁₀-alkenylene, C₂-C₁₀-haloalkenylene, C₂-C₁₀-alkynylene, C₂-C₁₀-haloalkynylene, C₃-C₈-cycloalkylene and C₃-C₈-halocycloalkylene, where the 8 aforementioned radicals can be substituted with one or more radicals R¹⁵;
A is selected from the group consisting of hydrogen, halogen, cyano, nitro, —SF₅, —SCN; C₁-C₆-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₆-alkenyl which may be partially or fully halogenated and/or may be substituted by one or more radicals R²⁰; C₂-C₆-alkynyl which may be partially or fully halogenated and/or may be substituted by one or more radicals R²⁰; phenyl which may be substituted by 1, 2, 3, 4 or 5 radicals R²⁴; a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴; —C(═O)R²⁰; —C(═O)OR²¹; —C(═O)N(R²²)R²³; —C(═S)R²⁰; —C(═S)OR²¹; —C(═S)N(R²²)R²³; —N(R²²)R²³; —NR²²C(═O)R²⁰; —NR²²C(═O)OR²¹; —N═SR³²R³³; —OR²¹; —SR²¹; —S(O)ₚR²¹; —S(O)ₙN(R²²)R²³; —CR^{d}═N—R^{c1}, —C(═N—N(R^{c1})R^{c2})R^{d}, —C(═NR^{c1})N(R^{c2})R^{c3}; —S(═N—R^{c1})R^{a}, —S(═O)(═N—R^{c1})—R^{a}, —C(═O)—N═S(R^{a})₂; —NR^{c1}—S—R^{a}, —NR^{c1}—S(═O)—R^{a}, —NR^{c1}—S(═O)₂—R^{a}; —NR^{c1}—S—N(R^{c2})R^{c3}; —NR^{c1}—S(═O)—N(R^{c2})R^{c3}, —NR^{c1}—S(═O)₂—N(R^{c2})R^{c3}; —N(═C(R^{d})R^{b}), and —N═(C(R^{d})N(R^{c1})R^{c2});
each B is independently selected from O, S, CR^{d1}R^{d2} and NR;
G is C—R¹⁴ or N;
Z is selected from phenyl which may carry 1, 2, 3, 4 or 5 radicals R^{4a}; a 5- or 6-membered heteroaromatic monocyclic ring and a 8-, 9- or 10-membered heteroaromatic bicyclic ring, the heteroaromatic mono- and bicyclic ring containing 1, 2, 3 or 4 heteroatoms selected from O, N and S as ring members, where the heteroaromatic mono- or bicyclic ring may carry 1, 2 or 3 radicals R^{4a};
Y is O, N—R^{y}, S(O)ₙ or a chemical bond;
R^{A} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₁-C₁₀-alkoxy; C₁-C₁₀-haloalkoxy; C₁-C₁₀-alkylthio; C₁-C₁₀-haloalkylthio; —C(═O)R²⁰; —C(═O)OR²¹; —C(═O)N(R²²)R²³; phenyl which may be substituted by 1, 2, 3, 4 or 5 radicals R²⁴; and a C-bound 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
R^{B} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰;
   —N(R²²)R²³; —N(R²²)C(═O)R²⁰; —OR²¹; —SR²¹; —S(O)ₚR²¹; —S(O)ₙN(R²²)R²³; —C(═O)R²⁰; —C(═O)OR²¹; —C(═O)N(R²²)R²³; —C(═S)R²⁰; —C(═S)OR²¹, —C(═S)N(R²²)R²³; —C(═NR²²)R²⁰; phenyl which may be substituted by 1, 2, 3, 4 or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
   with the proviso that R^{B} is not —OR²¹ if Y is O;
R^{y} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰;
   —N(R²²)R²³; —Si(R²⁶)₂R²⁵; —OR²¹; —SR²¹; —S(O)ₚR²¹; —S(O)ₙN(R²²)R²³; —C(═O)R²⁰; —C(═O)OR²¹; —C(═O)N(R²²)R²³; —C(═S)R²⁰; —C(═S)OR²¹; —C(═S)N(R²²)R²³; —C(═NR²²)R²⁰; phenyl which may be substituted by 1, 2, 3, 4 or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
   or R^{B} and R^{y} together form a group ═NR²² or ═NOR²¹;
   or R^{B} and R^{y} together form a C₂-C₇ alkylene chain, thus forming, together with the nitrogen atom to which they are bound, a 3-, 4-, 5-, 6-, 7- or 8-membered ring, where the alkylene chain may be interrupted by 1 or 2 O, S and/or NR²⁹ and/or 1 or 2 of the CH₂ groups of the alkylene chain may be replaced by a group C═O, C═S and/or C═NR²⁹; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
each R^{a} is independently selected from the group consisting of hydrogen, C₁-C₆-alkyl, C₂-C₆-alkenyl, C₂-C₆-alkynyl, C₃-C₈-cycloalkyl, C₁-C₆-alkylsulfinyl, C₁-C₆-alkylsulfonyl, wherein the aliphatic and cycloaliphatic moieties in the 6 aforementioned radicals may be partially or fully halogenated and/or may carry 1 or 2 substituents selected from C₁-C₄-alkoxy and oxo;
   phenyl, benzyl and pyridyl, wherein the three last-mentioned radicals may carry one or more substituents selected from halogen, C₁-C₆-alkyl, C₁-C₆-haloalkyl, C₁-C₆-alkoxy, C₁-C₆-haloalkoxy and C₁-C₆-alkoxycarbonyl;
each R^{b} is independently selected from the group consisting of hydrogen, C₁-C₆-alkyl, C₂-C₆-alkenyl, C₂-C₆-alkynyl, C₃-C₈-cycloalkyl, C₁-C₆-alkoxy, C₁-C₆-alkylthio, C₁-C₆-alkylsulfinyl, C₁-C₆-alkylsulfonyl, wherein the aliphatic and cycloaliphatic moieties in the 8 aforementioned radicals may be partially or fully halogenated and/or may carry 1 or 2 substituents selected from C₁-C₄-alkoxy and oxo;
   phenyl, benzyl, pyridyl and phenoxy, wherein the four last-mentioned radicals may carry one or more substituents selected from halogen, C₁-C₆-alkyl, C₁-C₆-haloalkyl, C₁-C₆-alkoxy, C₁-C₆-haloalkoxy, C₁-C₆-alkoxycarbonyl, C₁-C₆-alkylamino and di-(C₁-C₆-alkyl)amino;
R^{c}, R^{c1}, R^{c2} and R^{c3}, independently of each other and independently of each occurrence, are selected from the group consisting of hydrogen, cyano, C₁-C₆-alkyl, C₂-C₆-alkenyl, C₂-C₆-alkynyl, C₃-C₈-cycloalkyl, C₁-C₆-alkoxy, C₁-C₆-alkylthio, C₁-C₆-alkylsulfinyl, C₁-C₆-alkylsulfonyl, wherein the aliphatic and cycloaliphatic moieties in the 8 last-mentioned radicals may be partially or fully halogenated and/or may carry 1 or 2 substituents R¹⁸;
   phenyl, benzyl, pyridyl and phenoxy, wherein the four last-mentioned radicals may carry one or more substituents selected from halogen, cyano, C₁-C₆-alkyl, C₁-C₆-haloalkyl, C₂-C₆-alkenyl, C₂-C₆-alkynyl, C₃-C₈-cycloalkyl, C₁-C₆-alkoxy, C₁-C₆-haloalkoxy, C₁-C₆-alkoxycarbonyl, C₁-C₆-alkylamino and di-(C₁-C₆-alkyl)amino; or
   R^{c1} and R^{c2}, or R^{c2} and R^{c3}, together with the nitrogen atom to which they are bound, form a 3-, 4-, 5-, 6- or 7-membered saturated, partially unsaturated or maximally unsaturated heterocyclic ring which may additionally contain 1 or 2 further heteroatoms or heteroatom groups selected from N, O, S, NO, SO and SO₂ as ring members, where the heterocyclic ring may be substituted with one or more substituents selected from halogen, C₁-C₄-haloalkyl, C₁-C₄-alkoxy and C₁-C₄-haloalkoxy;
R^{d}, R^{d1} and R^{d2}, independently of each other and independently of each occurrence, are selected from the group consisting of hydrogen, halogen, cyano, nitro, —OH, —SH, —SCN, C₁-C₆-alkyl, C₂-C₆-alkenyl, C₂-C₆-alkynyl, C₃-C₈-cycloalkyl, C₁-C₆-alkoxy, C₁-C₆-alkylthio, C₁-C₆-alkylsulfinyl, C₁-C₆-alkylsulfonyl, wherein the aliphatic and cycloaliphatic moieties in the 8 last-mentioned radicals may be partially or fully halogenated and/or may carry 1 or 2 substituents selected from C₁-C₄-alkoxy and oxo;
   —OR^{a}, —NR^{c1}R^{c2}, —S(O)ₙR^{a}, —S(O)ₙNR^{c1}R^{c2}, —C(═O)R^{b}, —C(═O)NR^{c1}R^{c2}, —C(═O)OR^{a}, —C(═S)R^{b}, —C(═S)NR^{c1}R^{c2}, —C(═S)OR^{a}, —C(═S)SR^{a}, —C(═NR^{c1})R^{b}, —C(═NR^{c1})NR^{c2}R^{c3}, phenyl, benzyl, pyridyl and phenoxy, wherein the four last-mentioned radicals may carry one or more substituents selected from halogen, C₁-C₆-alkyl, C₁-C₆-haloalkyl, C₁-C₆-alkoxy and C₁-C₆-haloalkoxy;
R^{1a} is selected from hydrogen, cyano, C₁-C₆-alkyl, C₁-C₆-haloalkyl, C₃-C₆-cycloalkyl, C₃-C₆-halocycloalkyl, carboxyl, —C(═O)R^{5a}, —C(═O)OR^{6a} and —C(═O)NR^{7a}R^{8a};
R^{1b} is selected from hydrogen and C₁-C₆-alkyl;
R^{2a} and R^{2c}, independently of each other and independently of each occurrence, are selected from the group consisting of hydrogen, halogen, cyano, —C(═O)R⁵, —C(═S)R⁵, —C(═O)OR⁶, —C(═O)NR⁷R⁸; —C(═S)NR⁷R⁸, S(O)ₙR¹¹, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷;
R^{2b} and R^{2d}, independently of each other and independently of each occurrence, are selected from the group consisting of hydrogen, halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷; phenyl which may carry 1, 2 or 3 substituents R⁴; and a 5- or 6-membered heteroaromatic ring containing 1, 2 or 3 heteroatoms selected from O, N and S as ring members, where the heteroaromatic ring may carry 1, 2 or 3 substituents R⁴;
R^{3a} and R^{3c}, independently of each other and independently of each occurrence, are selected from the group consisting of hydrogen, halogen, cyano, —C(═O)R⁵, —C(═S)R⁵, —C(═O)OR⁶, —C(═O)NR⁷R⁸; —C(═S)NR⁷R⁸, OR¹², S(O)ₙR¹¹, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷;
R^{3b}, R^{3d} and R^{3e}, independently of each other and independently of each occurrence, are selected from the group consisting of hydrogen, halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, OR¹², S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷; phenyl which may carry 1, 2 or 3 substituents R⁴; and a 5- or 6-membered heteroaromatic ring containing 1, 2 or 3 heteroatoms selected from O, N and S as ring members, where the heteroaromatic ring may carry 1, 2 or 3 substituents R⁴;
   or R^{2a} and R^{2b}, or R^{2a} and R^{2c}, or R^{2c} and R^{2d}, or R^{3a} and R^{3b}, or R^{3a} and R^{3c}, or R^{3c} and R^{3d}, or R^{2a} and R^{3a}, or R^{2a} and R^{3c}, or R^{2c} and R^{3c}, or R^{2a} and R^{3e}, or R^{2c} and R^{3e}, or R^{3a} and R^{3e}, or R^{3c} and R^{3e}, together with the carbon atoms they are bound to, may form a 3- 4-, 5- or 6-membered saturated, partially unsaturated or maximally unsaturated carbocyclic or heterocyclic ring containing 1, 2 or 3 heteroatoms or heteroatom groups selected from N, O, S, NO, SO, SO₂, C(O) and C(S) as ring members, where the carbocyclic or heterocyclic ring may carry 1, 2 or 3 substituents selected from halogen, cyano, C₁-C₄-alkyl, C₁-C₄-haloalkyl, C₁-C₄-alkoxy and C₁-C₄-haloalkoxy;
R⁴ and R^{4a}, independently of each other and independently of each occurrence, are selected from the group consisting of halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, OR¹², S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
   or two radicals R⁴ or two radical R^{4a}, when bound to adjacent carbon atoms, together with these carbon atoms may form a 3-, 4-, 5- or 6-membered saturated, partially unsaturated or maximally unsaturated carbocyclic or heterocyclic ring containing 1, 2 or 3 heteroatoms or heteroatom groups selected from N, O, S, NO, SO, SO₂, C(O) and C(S) as ring members, where the carbocyclic or heterocyclic ring may carry 1, 2 or 3 substituents selected from halogen, cyano, C₁-C₄-alkyl, C₁-C₄-haloalkyl, C₁-C₄-alkoxy and C₁-C₄-haloalkoxy;
each R⁵ is independently selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{5a} is independently selected from the group consisting of hydrogen, C₁-C₆-alkyl, C₁-C₆-haloalkyl, C₃-C₈-cycloalkyl and C₃-C₈-halocycloalkyl;
each R⁶ is independently selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{6a} is independently selected from the group consisting of hydrogen, C₁-C₆-alkyl and C₁-C₆-haloalkyl;
R⁷ and R⁸, independently of each other and independently of each occurrence, are selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
   or R⁷ and R⁸, together with the nitrogen atom they are bound to, form a 5-, 6- or 7-membered saturated, partially unsaturated or maximally unsaturated heterocyclic ring, where the heterocyclic ring may additionally contain 1 or 2 heteroatoms or heteroatom groups selected from O, N, S, NO, SO, SO₂, C(O) and C(S) as ring members; where the heterocyclic ring may carry 1, 2 or 3 substituents R¹³;
R^{7a} and R^{8a}, independently of each other and independently of each occurrence, are selected from the group consisting of hydrogen, C₁-C₆-alkyl and C₁-C₆-haloalkyl;
R⁹ and R¹⁰, independently of each other and independently of each occurrence, are selected from the group consisting of hydrogen, —C(═O)R⁵, —C(═S)R⁵, —C(═O)OR⁶, —C(═O)NR⁷R⁸; —C(═S)NR⁷R⁸, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
   or R⁹ and R¹⁰, together with the nitrogen atom they are bound to, form a 5-, 6- or 7-membered saturated, partially unsaturated or maximally unsaturated heterocyclic ring, where the heterocyclic ring may additionally contain 1 or 2 heteroatoms or heteroatom groups selected from O, N, S, NO, SO, SO₂, C(O) and C(S) as ring members; where the heterocyclic ring may carry 1, 2 or 3 substituents R¹³;
R^{9a} and R^{10a}, independently of each other and independently of each occurrence, are selected from the group consisting of hydrogen, —C(═O)R^{5a}, —C(═S)R^{5a}, —C(═O)OR^{6a}, —C(═O)NR^{7a}R^{8a}; —C(═S)NR^{7a}R^{8a}, C₁-C₆-alkyl and C₁-C₆-haloalkyl;
each R¹¹ is independently selected from C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{11a} is independently selected from C₁-C₆-alkyl and C₁-C₆-haloalkyl;
each R¹² is independently selected from hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{12a} is independently selected from hydrogen, C₁-C₆-alkyl and C₁-C₆-haloalkyl;
each R¹³ is independently selected from the group consisting of halogen, cyano, nitro, —C(═O)R^{5a}, —C(═S)R^{5a}, —C(═O)OR^{6a}, —C(═O)NR^{7a}R^{8a}; —C(═S)NR^{7a}R^{8a}, NR^{9a}R^{10a}, OR^{12a}, S(O)ₙR^{11a}, S(O)₂NR^{9a}R^{10a}, C₁-C₆-alkyl, C₂-C₆-alkenyl, C₂-C₆-alkynyl, C₃-C₈-cycloalkyl and C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷;
   or two radicals R¹³, when bound to adjacent carbon atoms, together with these carbon atoms may form a 3- 4-, 5- or 6-membered saturated, partially unsaturated or maximally unsaturated carbocyclic or heterocyclic ring containing 1, 2 or 3 heteroatoms or heteroatom groups selected from N, O, S, NO, SO, SO₂, C(O) and C(S) as ring members, where the carbocyclic or heterocyclic ring may carry 1, 2 or 3 substituents selected from halogen, cyano, C₁-C₄-alkyl, C₁-C₄-haloalkyl, C₁-C₄-alkoxy and C₁-C₄-haloalkoxy;
R¹⁴ is selected from the group consisting of hydrogen, halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, OR¹², S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl and C₃-C₈-cycloalkenyl, wherein the aliphatic and cycloaliphatic moieties in the 6 last-mentioned radicals may be partially or fully halogenated and/or may carry 1 or 2 substituents R¹⁷;
each R¹⁵ is independently selected from cyano, nitro, —OH, —SH, —SCN, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-dialkylamino, C₁-C₆-alkylaminocarbonyl, C₁-C₆-dialkylaminocarbonyl, phenyl which may carry 1, 2, 3, 4 or 5 radicals R¹⁶; and a 3-, 4-, 5-, 6-, 7- or 8-membered saturated, partially unsaturated or maximally unsaturated heterocyclic ring containing 1, 2, 3 or 4 heteroatoms or heteroatom groups selected from N, S, O, NO, SO and SO₂ and optionally also 1 or 2 groups C(═O) or C(═S) as ring members, where the heterocyclic ring may be substituted by one or more radicals R¹⁶; and
   as a substituent on a cycloalkylene or halocycloalkylene moiety, R¹⁵ is additionally selected from C₁-C₆-alkyl, C₁-C₆-haloalkyl, C₂-C₆-alkenyl, C₂-C₆-haloalkenyl, C₂-C₆-alkynyl and C₂-C₆-haloalkynyl;
each R¹⁶ is independently selected from halogen, nitro, C₁-C₄-alkyl, C₁-C₄-haloalkyl, C₁-C₄-alkoxy, C₁-C₄-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-dialkylamino, C₁-C₆-alkylaminocarbonyl and C₁-C₆-dialkylaminocarbonyl;
each R¹⁷ is independently selected from cyano, nitro, OR^{12a} and S(O)ₙR^{11a}; and as a substituent on a cycloalkyl, cycloalkyl-alkyl or cycloalkenyl moiety, R¹⁷ is additionally selected from C₁-C₄-alkyl and C₁-C₄-haloalkyl;
each R¹⁸ is independently selected from cyano, nitro, OR^{12a}, oxo, S(O)ₙR^{11a}, C₃-C₈-cycloalkyl, C₃-C₈-halocycloalkyl, where the cycloaliphatic moieties in the two last-mentioned radicals may be substituted by one or more radicals selected oxo and cyano; C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-dialkylamino, C₁-C₆-alkylaminocarbonyl, C₁-C₆-dialkylaminocarbonyl, phenyl which may carry 1, 2, 3, 4 or 5 radicals R¹⁶; and a 3-, 4-, 5-, 6-, 7- or 8-membered saturated, partially unsaturated or maximally unsaturated heterocyclic ring containing 1, 2, 3 or 4 heteroatoms or heteroatom groups selected from N, S, O, NO, SO and SO₂ and optionally also 1 or 2 groups C(═O) or C(═S) as ring members, where the heterocyclic ring may be substituted by one or more radicals R¹⁶;
   and as a substituent on a cycloalkyl moiety, R¹⁸ is additionally selected from C₁-C₄-alkyl and C₁-C₄-haloalkyl;
each R²⁰ is independently selected from the group consisting of cyano, azido, nitro, —SCN, SF₅, C₃-C₈-cycloalkyl, C₃-C₈-halocycloalkyl, —Si(R²⁶)₂R²⁵, —OR²¹, —OSO₂R²¹, —SR²¹, —S(O)ₚR²¹, —S(O)ₙN(R²²)R²³, —N(R²²)R²³, —C(═O)N(R²²)R²³, —C(═S)N(R²²)R²³, —C(═O)OR²¹, —C(═O)R³⁰, phenyl which may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
   and, in case R²⁰ is bound to a cycloalkyl group or to a heterocyclic ring formed by R^{A} and R^{B} together with the atoms to which they are bound, R²⁰ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl in which the phenyl moiety may be substituted by 1, 2, 3, 4 or 5 radicals R²⁴;
   and in groups —C(═O)R²⁰, —C(═S)R²⁰, —C(═NR²²)R²⁰ and —N(R²²)C(═O)R²⁰, R²⁰ may additionally be selected from hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl in which the phenyl moiety may be substituted by 1, 2, 3, 4 or 5 radicals R²⁴;
   or two geminally bound radicals R²⁰ together form a group selected from ═CR²⁷R²⁸, ═S(O)ₘR²¹, ═S(O)ₘN(R²²)R²³, ═NR²², ═NOR²¹ and ═NNR²²;
   or two radicals R²⁰, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R²¹ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R²⁶)₂R²⁵, —SR³¹, —S(O)ₚR³¹, —S(O)ₙN(R²²)R²³, —N(R²²)R²³, —N═CR³²R³³, —C(═O)R³⁴, —C(═O)N(R²²)R²³, —C(═S)N(R²²)R²³, —C(═O)OR³⁴, phenyl which may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
   with the proviso that R²¹ is not C₁-C₆-alkoxy or C₁-C₆-haloalkoxy if it is bound to an oxygen atom;
R²² and R²³, independently of each other and independently of each occurrence, are selected from the group consisting of hydrogen, cyano, C₁-C₆-alkyl which may be partially or fully halogenated and/or may be substituted by one or more radicals R³⁰, C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R³⁰, C₃-C₈-cycloalkyl which may be partially or fully halogenated and/or may be substituted by one or more radicals R³⁰, C₃-C₈-cycloalkyl-C₁-C₄-alkyl where the cycloalkyl moiety may be partially or fully halogenated and/or may be substituted by one or more radicals R³⁰, C₂-C₆-alkenyl which may be partially or fully halogenated and/or may be substituted by one or more radicals R³⁰, C₂-C₆-alkynyl which may be partially or fully halogenated and/or may be substituted by one or more radicals R³⁰, —S(O)ₚR³¹, —S(O)ₙN(R³⁵)R³⁶, phenyl which may be substituted by 1, 2, 3, 4 or 5 radicals R²⁴, benzyl in which the phenyl moiety may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
   or R²² and R²³ together form a group ═CR²⁷R²⁸;
   or R²² and R²³, together with the nitrogen atom to which they are bound, may form a 3-, 4-, 5-, 6- or 7-membered saturated, partially unsaturated or maximally unsaturated heterocyclic ring which may additionally containing 1 or 2 further heteroatoms or heteroatom groups selected from N, O, S, NO, SO and SO₂, as ring members, where the heterocyclic ring may be substituted by one or more radicals R²⁴;
each R²⁴ is independently selected from the group consisting of halogen, cyano, azido, nitro, —SCN, SF₅, C₁-C₁₀-alkyl which may be partially or fully halogenated and/or may be substituted by one or more radicals R³⁰, C₃-C₈-cycloalkyl which may be partially or fully halogenated and/or may be substituted by one or more radicals R³⁰, C₂-C₁₀-alkenyl which may be partially or fully halogenated and/or may be substituted by one or more radicals R³⁰, C₂-C₁₀-alkynyl which may be partially or fully halogenated and/or may be substituted by one or more radicals R³⁰, —Si(R²⁶)₂R²⁵, —OR³¹, —OS(O)ₙR³¹, —SR³¹, —S(O)ₚR³¹, —S(O)ₙN(R³⁵)R³⁶, —N(R³⁵)R³⁶, C(═O)R³⁰, —C(═O)OR³¹, —C(═NR³⁵)R³⁶, —C(═O)N(R³⁵)R³⁶, —C(═S)N(R³⁵)R³⁶, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;
   or two radicals R²⁴ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR³⁵—, —CH₂CH═N—, —CH═CH—NR³⁵—, —OCH═N— and —SCH═N—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R²⁵ and R²⁶, independently of each other and independently of each occurrence, are selected from the group consisting of C₁-C₄-alkyl, C₃-C₆-cycloalkyl, C₁-C₄-alkoxy-C₁-C₄-alkyl, phenyl and benzyl;
R²⁷ and R²⁸, independently of each other and independently of each occurrence, are selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, —C(═O)R³⁰, —C(═O)OR³¹, —C(═NR³⁵)R³⁶, —C(═O)N(R³⁵)R³⁶, —C(═S)N(R³⁵)R³⁶, phenyl which may be substituted by 1, 2, 3, 4, or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R²⁴;
each R²⁹ is independently defined like R^{y};
each R³⁰ is independently selected from the group consisting of cyano, azido, nitro, —SCN, SF₅, C₃-C₈-cycloalkyl, C₃-C₈-halocycloalkyl, —Si(R²⁶)₂R²⁵, —OR³¹, —OSO₂R³¹, —SR³¹, —S(O)ₚR³¹, —S(O)ₙN(R³⁵)R³⁶, —N(R³⁵)R³⁶, —C(═O)N(R³⁵)R³⁶, —C(═S)N(R³⁵)R³⁶, —C(═O)OR³¹, —C(═O)R³¹, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   and, in case R³⁰ is bound to a cycloalkyl group, R³⁰ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl and C₂-C₆-haloalkynyl; and in groups —C(═O)R³⁰, R³⁰ may additionally be selected from hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, and C₂-C₆-haloalkynyl;
   or two geminally bound radicals R³⁰ together form a group selected from ═CR³⁷R³⁸, ═S(O)ₘR³¹, ═S(O)ₘN(R³⁵)R³⁶, ═NR³⁵, ═NOR³¹ and ═NNR³⁵;
   or two radicals R³⁰, together with the carbon atoms to which they are bound, form a 3-, 4-, 5-, 6-, 7- or 8-membered saturated or partially unsaturated carbocyclic or heterocyclic ring, where the heterocyclic ring contains 1, 2 or 3 heteroatoms or heteroatom groups selected from N, O, S, NO, SO and SO₂, as ring members;
each R³¹ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R²⁶)₂R²⁵, C₁-C₆-alkylaminosulfonyl, amino, C₁-C₆-alkylamino, di-(C₁-C₆-alkyl)amino, C₁-C₆-alkylcarbonyl, C₁-C₆-haloalkylcarbonyl, aminocarbonyl, C₁-C₆-alkylaminocarbonyl, di-(C₁-C₆-alkyl)-aminocarbonyl, C₁-C₆-alkoxycarbonyl, C₁-C₆-haloalkoxycarbonyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; benzyl in which the phenyl moiety may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   with the proviso that R³¹ is not C₁-C₆-alkoxy or C₁-C₆-haloalkoxy if it is bound to an oxygen atom;
R³² and R³³, independently of each other and independently of each occurrence, are selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R²⁴;
   with the proviso that R³² and R³³ are not hydrogen if bound to a sulfur atom (i.e. in —N═SR³²R³³, R³² and R³³ are not hydrogen);
each R³⁴ is independently selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl and benzyl; R³⁵ and R³⁶, independently of each other and independently of each occurrence, are selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; benzyl in which the phenyl moiety may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   or R³⁵ and R³⁶, together with the nitrogen atom to which they are bound, may form a 3-, 4-, 5-, 6- or 7-membered saturated, partially unsaturated or maximally unsaturated heterocyclic ring which may additionally contain 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy;
R³⁷ and R³⁸, independently of each other and independently of each occurrence, are selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, phenyl which may be substituted by 1, 2, 3, 4, or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R²⁴;
m is 0 or 1;
n is 0, 1 or 2; and
p is 1 or 2;
or the N-oxides or the tautomers or the agriculturally acceptable salts thereof.

The present invention also provides an agricultural composition comprising at least one compound of the formula I as defined herein, a stereoisomer thereof and/or at least one agriculturally acceptable salt thereof and at least one inert liquid and/or solid agriculturally acceptable carrier.

The present invention also provides a veterinary composition comprising at least one compound of the formula I as defined herein, a stereoisomer thereof and/or at least one veterinarily acceptable salt thereof and at least one inert liquid and/or solid veterinarily acceptable carrier.

The present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I, a stereoisomer thereof and/or at least one agriculturally acceptable salt thereof as defined herein.

The method serves in particular for protecting plants from attack or infestation by invertebrate pests, and thus comprises treating the plants with a pesticidally effective amount of at least one compound of the formula I as defined above, a stereoisomer thereof and/or at least one agriculturally acceptable salt thereof. The method further serves in particular for protecting plant propagation material and/or the plants which grow therefrom from attack or infestation by invertebrate pests, and thus comprises treating the plant propagation material with a pesticidally effective amount of at least one compound of the formula I as defined above, a stereoisomer thereof and/or at least one agriculturally acceptable salt thereof.

The present invention also relates to plant propagation material, in particular seed, comprising at least one compound of formula I, a stereoisomer thereof and/or at least one agriculturally acceptable salt thereof as defined herein.

The present invention further relates to a method for treating or protecting an animal from infestation or infection by parasites (invertebrate pests) which comprises bringing the animal in contact with a parasiticidally/pesticidally effective amount of a compound of the formula I, a stereoisomer thereof and/or at least one veterinarily acceptable salt thereof as defined herein. Bringing the animal in contact with the compound I, its salt or the veterinary composition of the invention means applying or administering it to the animal.

The present invention further relates to compounds of the formula I, stereoisomers thereof and/or veterinarily acceptable salts thereof as defined herein for use as a medicament, especially for use as a medicament for treating or protecting an animal from infestation or infection by parasites (invertebrate pests).

The term “stereoisomers” encompasses both optical isomers, such as enantiomers or diastereomers, the latter existing due to more than one center of chirality in the molecule, as well as geometrical isomers (cis/trans isomers).

The compounds I can be present as cis as well as trans isomers relating to the relative position of the group —Y—R^{B} to the azole ring containing the ring member G. These possible cis/trans stereoisomers are represented by the zigzag line between N and Y. In a specific embodiment, the group —Y—R^{B} is trans to the azole ring containing the ring member G (or, alternatively expressed, is cis to R^{A}).

Depending on the substitution pattern, the compounds of the formula I may have one or more centers of chirality, in which case they are present as mixtures of enantiomers or diastereomers. One center of chirality is for example the carbon atom carrying R^{1a}, R^{1b} and Q (if R^{1a} and R^{1b} are different). Also, the ring carbon atoms carrying R^{2a} and R^{2b} or R^{2c} and R^{2d} or the carbon atom(s) of the groups J¹ and J² or the carbon atom of X may form centers of chirality if suitably substituted. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term N-oxides relates to a form of compounds I in which at least one nitrogen atom is present in oxidized form (as NO). To be more precise, it relates to any compound of the present invention which has at least one tertiary nitrogen atom that is oxidized to an N-oxide moiety. N-oxides of compounds I can in particular be prepared by oxidizing e.g. the ring nitrogen atom of Q or X, and/or of any nitrogen-containing heterocyclic group present in group A, Z, R^{A} or R^{B} with a suitable oxidizing agent, such as peroxo carboxylic acids or other peroxides. The person skilled in the art knows if and in which positions compounds of the present invention may form N-oxides.

The compounds of the present invention may be amorphous or may exist in one ore more different crystalline states (polymorphs) which may have a different macroscopic properties such as stability or show different biological properties such as activities. The present invention includes both amorphous and crystalline compounds of the formula I, mixtures of different crystalline states of the respective compound I, as well as amorphous or crystalline salts thereof.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorids, sulphates, phosphates, and nitrates and salts of organic acids for example acetic acid, maleic acid, dimaleic acid, fumaric acid, difumaric acid, methane sulfenic acid, methane sulfonic acid, and succinic acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. The plant propagation materials may be treated prophylactically with a plant protection compound either at or before planting or transplanting. Said young plants may also be protected before transplantation by a total or partial treatment by immersion or pouring.

The term “plants” comprises any types of plants including “non-cultivated plants” and in particular “cultivated plants”.

The term “non-cultivated plants” refers to any wild type species or related species or related genera of a cultivated plant.

The term “cultivated plants” is to be understood as including plants which have been modified by breeding, mutagenesis or genetic engineering including but not limiting to agricultural biotech products on the market or in development (cf. http://www.bio.org). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibittors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink®(glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); BtXtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “alkyl” as used herein and in the alkyl moieties of alkoxy, alkylthio, alkylsulfinyl, alkylsulfonyl, alkylcarbonyl, alkoxycarbonyl and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”), 1 to 6 (“C₁-C₆-alkyl”), 1 to 8 (“C₁-C₈-alkyl”) or 1 to 10 (“C₁-C₁₀-alkyl”) carbon atoms. C₁-C₂-Alkyl is methyl or ethyl. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl and positional isomers thereof.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”), 1 to 6 (“C₁-C₆-haloalkyl”), 1 to 8 (“C₁-C₈-haloalkyl”) or 1 to 10 (“C₁-C₁₀-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like.

“Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”), 2 to 6 (“C₂-C₆-alkenyl”), 2 to 8 (“C₂-C₈-alkenyl”) or 2 to 10 (“C₂-C₁₀-alkenyl”) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “haloalkenyl” as used herein, which is also expressed as “alkenyl which is partially or fully halogenated”, refers to unsaturated straight-chain or branched hydrocarbon radicals having 2 to 3 (“C₂-C₃-haloalkenyl”), 2 to 4 (“C₂-C₄-haloalkenyl”), 2 to 6 (“C₂-C₆-haloalkenyl”), 2 to 8 (“C₂-C₆-haloalkenyl”) or 2 to 10 (“C₂-C₁₀-haloalkenyl”) carbon atoms and a double bond in any position (as mentioned above), where some or all of the hydrogen atoms in these groups are replaced by halogen atoms as mentioned above, in particular fluorine, chlorine and bromine, for example chlorovinyl, chloroallyl and the like.

The term “alkynyl” as used herein refers to straight-chain or branched hydrocarbon groups having 2 to 3 (“C₂-C₃-alkynyl”), 2 to 4 (“C₂-C₄-alkynyl”), 2 to 6 (“C₂-C₆-alkynyl”), 2 to 8 (“C₂-C₈-alkynyl”), or 2 to 10 (“C₂-C₁₀-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “haloalkynyl” as used herein, which is also expressed as “alkynyl which is partially or fully halogenated”, refers to unsaturated straight-chain or branched hydrocarbon radicals having 2 to 3 (“C₂-C₃-haloalkynyl”), 2 to 4 (“C₂-C₄-haloalkynyl”), 3 to 4 (“C₃-C₄-haloalkynyl”), 2 to 6 (“C₂-C₆-haloalkynyl”), 2 to 8 (“C₂-C₈-haloalkynyl”) or 2 to 10 (“C₂-C₁₀-haloalkynyl”) carbon atoms and one or two triple bonds in any position (as mentioned above), where some or all of the hydrogen atoms in these groups are replaced by halogen atoms as mentioned above, in particular fluorine, chlorine and bromine;

The term “cycloalkyl” as used herein refers to mono- or bi- or polycyclic saturated hydrocarbon radicals having 3 to 8 (“C₃-C₈-cycloalkyl”), in particular 3 to 6 (“C₃-C₆-cycloalkyl”) or 3 to 5 (“C₃-C₅-cycloalkyl”) or 3 to 4 (“C₃-C₄-cycloalkyl”) carbon atoms. Examples of monocyclic radicals having 3 to 4 carbon atoms comprise cyclopropyl and cyclobutyl. Examples of monocyclic radicals having 3 to 5 carbon atoms comprise cyclopropyl, cyclobutyl and cyclopentyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “halocycloalkyl” as used herein, which is also expressed as “cycloalkyl which is partially or fully halogenated”, refers to mono- or bi- or polycyclic saturated hydrocarbon groups having 3 to 8 (“C₃-C₈-halocycloalkyl”) or preferably 3 to 6 (“C₃-C₆-halocycloalkyl”) or 3 to 5 (“C₃-C₅-halocycloalkyl”) or 3 to 4 (“C₃-C₄-halocycloalkyl”) carbon ring members (as mentioned above) in which some or all of the hydrogen atoms are replaced by halogen atoms as mentioned above, in particular fluorine, chlorine and bromine.

The term “cycloalkyl-C₁-C₄-alkyl” refers to a C₃-C₈-cycloalkyl group (“C₃-C₈-cycloalkyl-C₁-C₄-alkyl”), preferably a C₃-C₆-cycloalkyl group (“C₃-C₆-cycloalkyl-C₁-C₄-alkyl”), more preferably a C₃-C₄-cycloalkyl group (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C₁-C₄-alkyl, are cyclopentylmethyl, cyclopentylethyl, cyclopentylpropyl, cyclohexylmethyl, cyclohexylethyl and cyclohexylpropyl. Examples for C₃-C₈-cycloalkyl-C₁-C₄-alkyl, apart those mentioned for C₃-C₆-cycloalkyl-C₁-C₄-alkyl, are cycloheptylmethyl, cycloheptylethyl, cyclooctylmethyl and the like.

The terms “C₃-C₈-halocycloalkyl-C₁-C₄-alkyl” and “C₃-C₆-halocycloalkyl-C₁-C₄-alkyl” refers to a C₃-C₈-halocycloalkyl or C₃-C₆-halocycloalkyl group as defined above which is bound to the remainder of the molecule via a C₁-C₄-alkyl group, as defined above.

The term “cycloalkenyl” as used herein refers to monocyclic hydrocarbon radicals with at least one C═C double bond in the ring, which ring is however not aromatic, the hydrocarbon radicals having 3 to 8 (“C₃-C₈-cycloalkyl) carbon atoms. Examples are cyclopropenyl, such as cycloprop-1-enyl and cycloprop-2-yl, cyclobutenyl, such as cyclobut-1-enyl and cyclobut-2-enyl, cyclopentenyl, such as cyclopent-1-enyl, cyclopent-2-enyl and cyclopent-3-enyl, cyclopentadienyl, such as cyclopenta-1,3-dienyl, cyclpenta-1,4-dienyl and cyclpenta-2,4-dienyl, cyclohexenyl, such as cyclohex-1-enyl, cyclohex-2-enyl and cyclohex-3-enyl, cyclohexadienyl, such as cyclohexa-1,3-dienyl, cyclohexa-1,4-dienyl, cyclohexa-1,5-dienyl and cyclohexa-2,5-dienyl, cycloheptenyl, cycloheptadienyl, cycloheptatrienyl cyclooctenyl, cyclooctadieny, cyclooctatrienyl and cyclooctatetraenyl.

The term “halocycloalkenyl” as used herein refers to monocyclic hydrocarbon radicals with at least one C—C double bond in the ring, which ring is however not aromatic, the hydrocarbon radicals having 3 to 8 (“C₃-C₈-halocycloalkyl”) carbon atoms, and wherein some or all of the hydrogen atoms are replaced by halogen atoms as mentioned above, in particular fluorine, chlorine and bromine.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is methoxy or ethoxy. C₁-C₃-Alkoxy is additionally, for example, n-propoxy and 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. The term “C₁-C₁₀-haloalkoxy” is a C₁-C₁₀-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₃-alkoxy-C₁-C₃-alkyl” as used herein, refers to a straight-chain or branched alkyl group having 1 to 3 carbon atoms, as defined above, where one hydrogen atom is replaced by a C₁-C₃-alkoxy group, as defined above. The term “C₁-C₄-alkoxy-C₁-C₄-alkyl” as used herein, refers to a straight-chain or branched alkyl group having 1 to 4 carbon atoms, as defined above, where one hydrogen atom is replaced by a C₁-C₄-alkoxy group, as defined above. The term “C₁-C₆-alkoxy-C₁-C₆-alkyl” as used herein, refers to a straight-chain or branched alkyl group having 1 to 6 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “C₁-C₄-alkoxy-methyl” as used herein, refers to methyl in which one hydrogen atom is replaced by a C₁-C₄-alkoxy group, as defined above. The term “C₁-C₆-alkoxy-methyl” as used herein, refers to methyl in which one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, pentyloxymethyl, hexyloxymethyl and the like.

C₁-C₆-Haloalkoxy-C₁-C₆-alkyl is a straight-chain or branched alkyl group having from 1 to 6, especially 1 to 4 carbon atoms (═C₁-C₆-haloalkoxy-C₁-C₄-alkyl), wherein one of the hydrogen atoms is replaced by a C₁-C₆-alkoxy group and wherein at least one, e.g. 1, 2, 3, 4 or all of the remaining hydrogen atoms (either in the alkoxy moiety or in the alkyl moiety or in both) are replaced by halogen atoms. C₁-C₄-Haloalkoxy-C₁-C₄-alkyl is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms (either in the alkoxy moiety or in the alkyl moiety or in both) are replaced by halogen atoms. Examples are difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoromethoxymethyl (CH₃OCF₂), 1,1-difluoro-2-methoxyethyl, 2,2-difluoro-2-methoxyethyl and the like.

The term “C₁-C₂-alkylthio” is a C₁-C₂-alkyl group, as defined above, attached via a sulfur atom. The term “C₁-C₃-alkylthio” is a C₁-C₃-alkyl group, as defined above, attached via a sulfur atom. The term “C₁-C₄-alkylthio” is a C₁-C₄-alkyl group, as defined above, attached via a sulfur atom. The term “C₁-C₆-alkylthio” is a C₁-C₆-alkyl group, as defined above, attached via a sulfur atom. The term “C₁-C₁₀-alkylthio” is a C₁-C₁₀-alkyl group, as defined above, attached via a sulfur atom. C₁-C₂-Alkylthio is methylthio or ethylthio. C₁-C₃-Alkylthio is additionally, for example, n-propylthio or 1-methylethylthio (isopropylthio). C₁-C₄-Alkylthio is additionally, for example, butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “C₁-C₂-haloalkylthio” is a C₁-C₂-haloalkyl group, as defined above, attached via a sulfur atom. The term “C₁-C₃-haloalkylthio” is a C₁-C₃-haloalkyl group, as defined above, attached via a sulfur atom. The term “C₁-C₄-haloalkylthio” is a C₁-C₄-haloalkyl group, as defined above, attached via a sulfur atom. The term “C₁-C₆-haloalkylthio” is a C₁-C₆-haloalkyl group, as defined above, attached via a sulfur atom. The term “C₁-C₁₀-haloalkylthio” is a C₁-C₁₀-haloalkyl group, as defined above, attached via a sulfur atom. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₃-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio or 1-(CH₂Br)-2-bromoethylthio. C₁-C₄-Haloalkylthio is additionally, for example, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The term “C₁-C₂-alkylsulfinyl” is a C₁-C₂-alkyl group, as defined above, attached via a sulfinyl [S(O)] group. The term “C₁-C₄-alkylsulfinyl” is a C₁-C₄-alkyl group, as defined above, attached via a sulfinyl [S(O)] group. The term “C₁-C₆-alkylsulfinyl” is a C₁-C₆-alkyl group, as defined above, attached via a sulfinyl [S(O)] group. The term “C₁-C₁₀-alkylsulfinyl” is a C₁-C₁₀-alkyl group, as defined above, attached via a sulfinyl [S(O)] group. C₁-C₂-Alkylsulfinyl is methylsulfinyl or ethylsulfinyl. C₁-C₄-Alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl) or 1,1-dimethylethylsulfinyl (tert-butylsulfinyl). C₁-C₆-Alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl. C₁-C₈-Alkylsulfinyl is additionally, for example, heptylsulfinyl, octylsulfinyl, 2-ethylhexylsulfinyl and positional isomers thereof. C₁-C₁₀-Alkylsulfinyl is additionally, for example, nonylsulfinyl, decylsulfinyl and positional isomers thereof.

The term “C₁-C₂-haloalkylsulfinyl” is a C₁-C₂-haloalkyl group, as defined above, attached via a sulfinyl [S(O)] group. The term “C₁-C₄-haloalkylsulfinyl” is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. The term “C₁-C₁₀-haloalkylsulfinyl” is a C₁-C₁₀-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHCl₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-Haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The term “C₁-C₂-alkylsulfonyl” is a C₁-C₂-alkyl group, as defined above, attached via a sulfonyl [S(O)₂] group. The term “C₁-C₃-alkylsulfonyl” is a C₁-C₃-alkyl group, as defined above, attached via a sulfonyl [S(O)₂] group. The term “C₁-C₄-alkylsulfonyl” is a C₁-C₄-alkyl group, as defined above, attached via a sulfonyl [S(O)₂] group. The term “C₁-C₆-alkylsulfonyl” is a C₁-C₆-alkyl group, as defined above, attached via a sulfonyl [S(O)₂] group. The term “C₁-C₁₀-alkylsulfonyl” is a C₁-C₁₀-alkyl group, as defined above, attached via a sulfonyl [S(O)₂] group. C₁-C₂-Alkylsulfonyl is methylsulfonyl or ethylsulfonyl. C₁-C₃-Alkylsulfonyl is additionally, for example, n-propylsulfonyl or 1-methylethylsulfonyl (isopropylsulfonyl). C₁-C₄-Alkylsulfonyl is additionally, for example, butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” is a C₁-C₂-haloalkyl group, as defined above, attached via a sulfonyl [S(O)₂] group. The term “C₁-C₃-haloalkylsulfonyl” is a C₁-C₃-haloalkyl group, as defined above, attached via a sulfonyl [S(O)₂] group. The term “C₁-C₄-haloalkylsulfonyl” is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. The term “C₁-C₁₀-haloalkylsulfonyl” is a C₁-C₁₀-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₃-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonylor 1-(CH₂Br)-2-bromoethylsulfonyl. C₁-C₄-Haloalkylsulfonyl is additionally, for example, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The substituent “oxo” replaces a CH₂ group by a C(═O) group.

“Carboxyl” is —C(O)OH.

The term “alkylcarbonyl” is a C₁-C₆-alkyl (“C₁-C₆-alkylcarbonyl”), preferably a C₁-C₄-alkyl (“C₁-C₄-alkylcarbonyl”) group, as defined above, attached via a carbonyl [C(═O)] group. Examples are acetyl (methylcarbonyl), propionyl (ethylcarbonyl), propylcarbonyl, isopropylcarbonyl, n-butylcarbonyl and the like.

The term “haloalkylcarbonyl” is a C₁-C₆-haloalkyl (“C₁-C₆-haloalkylcarbonyl”), preferably a C₁-C₄-haloalkyl (“C₁-C₄-haloalkylcarbonyl”) group, as defined above, attached via a carbonyl [C(═O)] group. Examples are trifluoromethylcarbonyl, 2,2,2-trifluoroethylcarbonyl and the like.

The term “alkoxycarbonyl” is a C₁-C₆-alkoxy (“C₁-C₆-alkoxycarbonyl”), preferably a C₁-C₄-alkoxy (“C₁-C₄-alkoxycarbonyl”) group, as defined above, attached via a carbonyl [C(═O)] group. Examples are methoxycarbonyl), ethoxycarbonyl, propoxycarbonyl, isopropoxycarbonyl, n-butoxycarbonyl and the like.

The term “haloalkoxycarbonyl” is a C₁-C₆-haloalkoxy (“C₁-C₆-haloalkoxycarbonyl”), preferably a C₁-C₄-haloalkoxy (“C₁-C₄-haloalkoxycarbonyl”) group, as defined above, attached via a carbonyl [C(═O)] group. Examples are trifluoromethoxycarbonyl, 2,2,2-trifluoroethoxycarbonyl and the like.

The term “C₁-C₆-alkylamino” is a group —N(H)C₁-C₆-alkyl. Examples are methylamino, ethylamino, propylamino, isopropylamino, butylamino and the like.

The term “di-(C₁-C₆-alkyl)amino” is a group —N(C₁-C₆-alkyl)₂. Examples are dimethylamino, diethylamino, ethylmethylamino, dipropylamino, diisopropylamino, methylpropylamino, methylisopropylamino, ethylpropylamino, ethylisopropylamino, dibutylamino and the like.

The term “C₁-C₆-alkylaminocarbonyl” is a group —C(O)—N(H)C₁-C₆-alkyl. Examples are methylaminocarbonyl, ethylaminocarbonyl, propylaminocarbonyl, isopropylaminocarbonyl, butylaminocarbonyl and the like.

The term “di-(C₁-C₆-alkyl)aminocarbonyl” is a group —C(O)—N(C₁-C₆-alkyl)₂. Examples are dimethylaminocarbonyl, diethylaminocarbonyl, ethylmethylaminocarbonyl, dipropylaminocarbonyl, diisopropylaminocarbonyl, methylpropylaminocarbonyl, methylisopropylaminocarbonyl, ethylpropylaminocarbonyl, ethylisopropylaminocarbonyl, dibutylaminocarbonyl and the like.

The term “C₁-C₆-alkylaminosulfonyl” is a group —S(O)₂—N(H)C₁-C₆-alkyl. Examples are methylaminosulfonyl, ethylaminosulfonyl, propylaminosulfonyl, isopropylaminosulfonyl, butylaminosulfonyl and the like.

C₂-C₄-Alkylene is a linear or branched divalent alkyl radical having 2, 3 or 4 carbon atoms. Examples are —CH₂CH₂—, —CH(CH₃)—, —CH₂CH₂CH₂—, —CH(CH₃)CH₂—, —CH₂CH(CH₃)—, —C(CH₃)₂—, —CH₂CH₂CH₂CH₂—, —CH(CH₃)CH₂CH₂—, —CH₂CH₂CH(CH₃)—, —C(CH₃)₂CH₂—, and —CH₂C(CH₃)₂—. C₁-C₄-Alkylene is a linear or branched divalent alkyl radical having 1, 2, 3 or 4 carbon atoms. Examples are the radicals stated above for C₂-C₄-alkylene and further —CH₂—. Linear or branched C₂-C₆-alkylene is a linear or branched divalent alkyl radical having 2, 3, 4, 5 or 6 carbon atoms. Examples, in addition to the radicals stated above for C₂-C₄-alkylene, are —CH₂CH₂CH₂CH₂CH₂—, —CH₂CH₂CH₂CH₂CH₂CH₂— and positional isomers thereof. C₁-C₆-Alkylene is a linear or branched divalent alkyl radical having 1, 2, 3, 4, 5 or 6 carbon atoms. Examples are the radicals stated above for C₂-C₆-alkylene and further —CH₂—. Linear or branched C₂-C₇-alkylene is a linear or branched divalent alkyl radical having 2, 3, 4, 5, 6 or 7 carbon atoms. Examples, in addition to the radicals stated above for C₂-C₆-alkylene, are —(CH₂)₇—, and positional isomers thereof. C₁-C₇-Alkylene is a linear or branched divalent alkyl radical having 1, 2, 3, 4, 5, 6 or 7 carbon atoms. Examples are the radicals stated above for C₂-C₇-alkylene and further —CH₂—. Linear or branched C₂-C₈-alkylene is a linear or branched divalent alkyl radical having 2, 3, 4, 5, 6, 7 or 8 carbon atoms. Examples, in addition to the radicals stated above for C₂-C₆-alkylene, are —(CH₂)₇—, —(CH₂)₈—, and positional isomers thereof. C₁-C₈-Alkylene is a linear or branched divalent alkyl radical having 1, 2, 3, 4, 5, 6, 7 or 8 carbon atoms. Examples are the radicals stated above for C₂-C₈-alkylene and further —CH₂—. Linear or branched C₂-C₁₀-alkylene is a linear or branched divalent alkyl radical having 2 to 10 carbon atoms. Examples, in addition to the radicals stated above for C₂-C₈-alkylene, are the higher homologs with 9 or 10 carbon atoms, such as —(CH₂)₉—, —(CH₂)₁₀—, and positional isomers thereof. C₁-C₁₀-Alkylene is a linear or branched divalent alkyl radical having 1, 2, 3, 4, 5, 6, 7, 8, 9 or 10 carbon atoms. Examples are the radicals stated above for C₂-C₁₀-alkylene and further —CH₂—. Linear or branched C₂-C₁₂-alkylene is a linear or branched divalent alkyl radical having 2 to 12 carbon atoms. Examples, in addition to the radicals stated above for C₂-C₁₀-alkylene, are the higher homologs with 11 or 12 carbon atoms, such as —(CH₂)₁₁—, —(CH₂)₁₂—, and positional isomers thereof. Linear or branched C₁-C₁₂-alkylene is a linear or branched divalent alkyl radical having 1 to 12 carbon atoms. Examples are the radicals stated above for C₂-C₁₂-alkylene and further —CH₂—.

Haloalkylene is a linear or branched divalent alkyl radical having 1 to 4 (C₁-C₄-haloalkylene) or 1 to 6 (C₁-C₆-haloalkylene) or 1 to 8 (C₁-C₈-haloalkylene) or 1 to 10 (C₁-C₁₀-haloalkylene) or 1 to 12 (C₁-C₁₂-haloalkylene) carbon atoms, in which a part or all of the hydrogen atoms are replaced by halogen atoms, especially F or Cl.

Alkenylene is a linear or branched aliphatic, singly or multiply, e.g., singly or doubly, olefinically unsaturated divalent radical having for example 2 to 12 (C₂-C₁₂-alkenylene) or 2 to 10 (C₂-C₁₀-alkenylene) or 2 to 8 (C₂-C₈-alkenylene) carbon atoms. If the radical contains more than one carbon-carbon double bond these bonds are preferably not vicinal, i.e., not allenic. Examples are —CH═CH—, —C(═CH₂)—, —CH₂—CH═CH—, —CH═CH—CH₂, —C(CH₃)═CH—, —CH═C(CH₃)—, —CH₂—CH₂—CH═CH—, —CH₂—CH═CH—CH₂—, —CH═CH—CH₂—CH₂—, —CH(CH₃)—CH═CH—, —CH═CH—CH═CH— and the like.

Haloalkenylene is an alkenylene radical in which a part or all of the hydrogen atoms are replaced by halogen atoms, especially F or Cl.

Alkynylene is a linear or branched aliphatic divalent radical having, for example, 2 to 12 (C₂-C₁₂-alkynylene) or 2 to 10 (C₂-C₁₀-alkynylene) or 2 to 8 (C₂-C₈-alkynylene) carbon atoms and containing one or more, e.g., 1 or 2, carbon-carbon triple bonds. Examples are —C≡C—, —CH₂—C≡C—, —C≡C—CH₂—, —CH₂—CH₂—C≡C—, —CH₂—C≡C—CH₂—, —C≡C—CH₂—CH₂—, —CH(CH₃)—C≡C—, —C≡C—C≡C— and the like.

Haloalkynylene is an alkynylene radical in which a part or all of the hydrogen atoms are replaced by halogen atoms, especially F or Cl.

C₃-C₈-Cycloalkylene stands for a divalent monocyclic, saturated hydrocarbon group having 3 to 8 carbon ring members. Examples are cyclopropane-1,1-diyl, cyclopropane-1,2-diyl, cyclobutane-1,1-diyl, cyclobutane-1,2-diyl, cyclobutane-1,3-diyl, cyclopentane-1,1-diyl, cyclopentane-1,2-diyl, cyclopentane-1,3-diyl, cyclohexane-1,1-diyl, cyclohexane-1,2-diyl, cyclohexane-1,3-diyl, cyclohexane-1,4-diyl, cycloheptane-1,1-diyl, cycloheptane-1,2-diyl, cycloheptane-1,3-diyl, cycloheptane-1,4-diyl, cyclooctane-1,1-diyl, cyclooctane-1,2-diyl, cyclooctane-1,3-diyl, cyclooctane-1,4-diyl, and cyclooctane-1,5-diyl.

Halocycloalkylene is a cycloalkylene radical in which a part or all of the hydrogen atoms are replaced by halogen atoms, especially F or Cl.

The term “3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members” denotes a 3-, 4-, 5-, 6- or 7-membered saturated, partially unsaturated or maximum unsaturated heteromonocyclic ring containing 1, 2 or 3 heteroatoms or heteroatom groups selected from N, O, S, NO, SO and SO₂, as ring members.

The term “3-, 4-, 5-, 6-, 7- or 8-membered saturated, partially unsaturated or maximally unsaturated heterocyclic ring containing 1, 2 or 3 (or 4) heteroatoms or heteroatom groups selected from N, O, S, NO, SO and SO₂, as ring members” [wherein “maximally (or maximum) unsaturated” includes also “aromatic” ] denotes a 3-, 4-, 5-, 6-, 7- or 8-membered saturated, partially unsaturated or maximum unsaturated heteromonocyclic ring or a 8-membered saturated, partially unsaturated or maximally unsaturated heterobicyclic ring containing 1, 2 or 3 (or 4) heteroatoms or heteroatom groups selected from N, O, S, NO, SO and SO₂, as ring members.

Unsaturated rings contain at least one C—C and/or C—N and/or N—N double bond(s). Maximally unsaturated rings contain as many conjugated C—C and/or C—N and/or N—N double bonds as allowed by the ring size. Maximally unsaturated 5- or 6-membered heterocyclic rings are aromatic. 7- and 8-membered rings cannot be aromatic. They are homoaromatic (7-membered ring, 3 double bonds) or are olefinic, having 4 double bonds (8-membered ring). The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent. If the heterocyclic ring is substituted, the substituent may be bound to a carbon or a nitrogen ring atom.

Examples of a 3-, 4-, 5-, 6- or 7-membered saturated heterocyclic ring include: Oxiranyl, thiiranyl, aziridinyl, diaziridinyl, oxetanyl, thietanyl, 1-oxothietanyl, 1,1-dioxothietanyl, azetidinyl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl, and the like. Examples of an 8-membered saturated heterocyclic ring include azocanyl, oxocanyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl. Examples of an 8-membered partially unsaturated heterocyclic ring include hexahydroazocine, tetrahydroazocine, dihydroazocine, hexahydrooxocine, tetrahydrooxocine, dihydrooxocine and the like.

Examples for a 3-, 4-, 5-, 6- or 7-membered maximally unsaturated (including aromatic) heterocyclic ring are 5- or 6-membered heteroaromatic rings, such as 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,2,3-triazol-1-yl, 1,2,3-triazol-2-yl, 1,2,3-triazol-4-yl, 1,3,4-triazol-1-yl, 1,3,4-triazol-2-yl, 1,3,4-triazol-3-yl, 1,2,3,4-tetrazol-1-yl, 1,2,3,4-tetrazol-2-yl, 1,2,3,4-tetrazol-5-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl, and also homoaromatic radicals, such as 1H-azepine, 1H-[1,3]-diazepine and 1H-[1,4]-diazepine. Examples for an 8-membered maximally unsaturated heterocyclic ring are azocine, diazocine and the like.

Examples for an 8-membered saturated heterobicyclic ring containing 1, 2 or 3 (or 4) heteroatoms or heteroatom groups selected from N, O, S, NO, SO and SO₂, as ring members are:

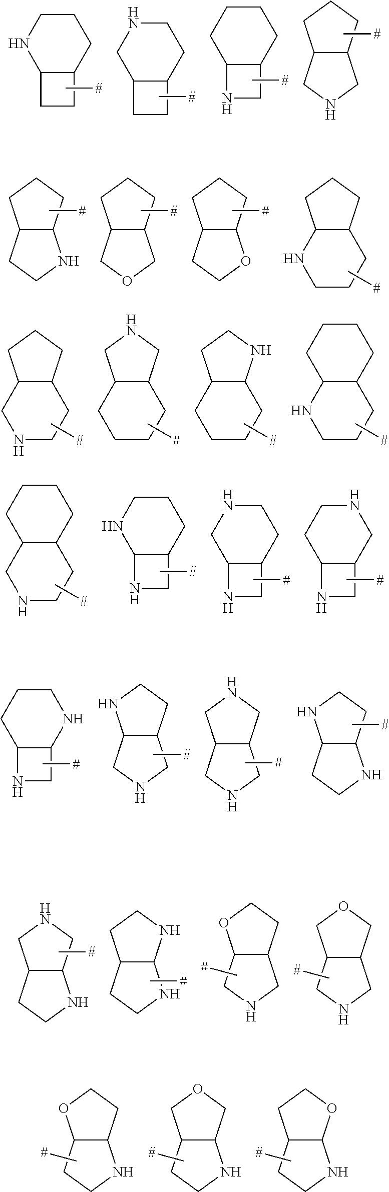

Examples for 5- or 6-membered heteroaromatic monocyclic rings containing 1, 2, 3 or 4 heteroatoms selected from O, N and S as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,2,3-triazol-1-yl, 1,2,3-triazol-2-yl, 1,2,3-triazol-4-yl, 1,3,4-triazol-1-yl, 1,3,4-triazol-2-yl, 1,3,4-triazol-3-yl, 1,2,3,4-tetrazol-1-yl, 1,2,3,4-tetrazol-2-yl, 1,2,3,4-tetrazol-5-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl.

Examples for a 8-, 9- or 10-membered heteroaromatic bicyclic ring containing 1, 2, 3 or 4 heteroatoms selected from O, N and S as ring members are:

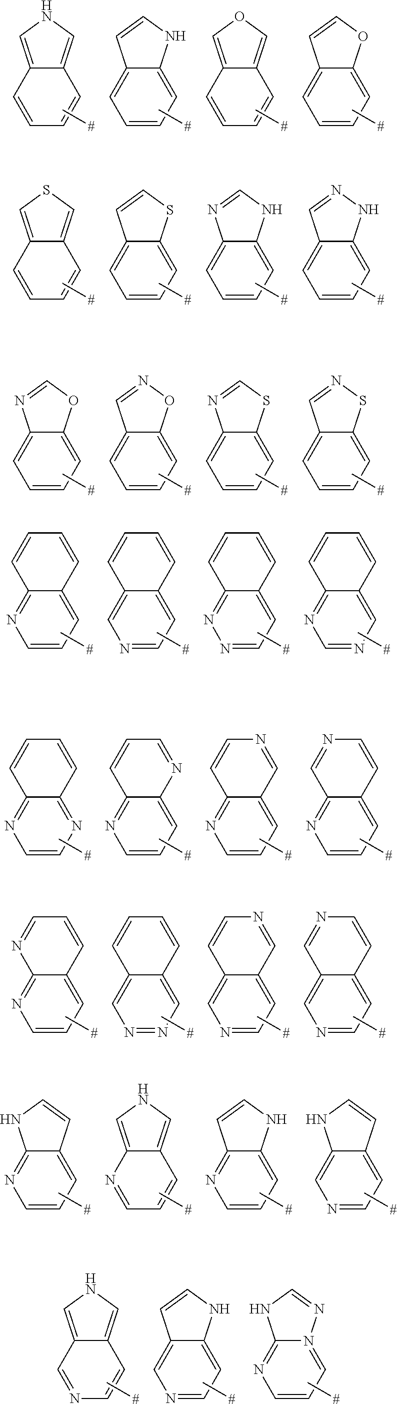

In the above structures # denotes the attachment point to the remainder of the molecule. The attachment point is not restricted to the ring on which is shown, but can be on either of the fused rings, and may be on a carbon or on a nitrogen ring atom. If the rings carry one or more substituents, these may be bound to carbon and/or to nitrogen ring atoms.

The term “3-, 4-, 5-, 6-, 7- or 8-membered saturated, partially unsaturated or maximally unsaturated heterocyclic ring containing 1, 2, 3 or 4 heteroatoms or heteroatom groups selected from N, S, O, NO, SO and SO₂ and optionally also 1 or 2 groups C(═O) or C(═S) as ring members” [wherein “maximally (or maximum) unsaturated” includes also “aromatic” ] denotes a 3-, 4-, 5-, 6-, 7- or 8-membered saturated, partially unsaturated or maximum unsaturated heteromonocyclic ring containing 1, 2 or 3 or 4 heteroatoms or heteroatom groups selected from N, O, S, NO, SO and SO₂, and optionally also 1 or 2 groups C(═O) or C(═S) as ring members. Examples therefor are the above-listed 3-, 4-, 5-, 6-, 7- or 8-membered saturated, partially unsaturated or maximally unsaturated heterocyclic ring containing 1, 2, 3 or 4 heteroatoms or heteroatom groups selected from N, S, O, NO, SO and SO₂ and further following rings: tetrahydrofuran-2-on-3-yl, tetrahydrofuran-2-on-4-yl, tetrahydrofuran-2-on-5-yl, tetrahydrofuran-2-thion-3-yl, tetrahydrofuran-2-thion-4-yl, tetrahydrofuran-2-thion-5-yl, pyrrolidin-2-on-1-yl, pyrrolidin-2-on-3-yl, pyrrolidin-2-on-4-yl, pyrrolidin-2-on-5-yl, pyrrolidin-2-thion-1-yl, pyrrolidin-2-thion-3-yl, pyrrolidin-2-thion-4-yl, pyrrolidin-2-thion-5-yl, pyrazolidin-3-on-1-yl, pyrazolidin-3-on-4-yl, pyrazolidin-3-on-5-yl, pyrazolidin-3-thion-1-yl, pyrazolidin-3-thion-4-yl, pyrazolidin-3-thion-5-yl, imidazolidin-2-on-1-yl, imidazolidin-2-on-4-yl, imidazolidin-4-on-1-yl, imidazolidin-4-on-2-yl, imidazolidin-4-on-3-yl, imidazolidin-4-on-5-yl, imidazolidin-2-thion-1-yl, imidazolidin-2-thion-4-yl, imidazolidin-4-thion-1-yl, imidazolidin-4-thion-2-yl, imidazolidin-4-thion-3-yl, imidazolidin-4-thion-5-yl, oxazolidin-2-on-3-yl, oxazolidin-2-on-4-yl, oxazolidin-2-thion-3-yl, oxazolidin-2-thion-4-yl, isoxazolidin-3-on-2-yl, isoxazolidin-3-on-4-yl, isoxazolidin-3-on-5-yl, isoxazolidin-3-thion-2-yl, isoxazolidin-3-thion-4-yl, isoxazolidin-3-thion-5-yl, tetrahydropyran-2-on-3-yl, tetrahydropyran-2-on-4-yl, tetrahydropyran-2-on-5-yl, tetrahydropyran-2-on-6-yl, tetrahydropyran-2-thion-3-yl, tetrahydropyran-2-thion-4-yl, tetrahydropyran-2-thion-5-yl, tetrahydropyran-2-thion-6-yl, 1,3-dioxan-2-on-4-yl, 1,3-dioxan-2-on-5-yl, 1,4-dioxan-2-on-3-yl, 1,4-dioxan-2-on-5-yl, 1,4-dioxan-2-on-6-yl, 1,3-dioxan-2-thion-4-yl, 1,3-dioxan-2-thion-5-yl, 1,4-dioxan-2-thion-3-yl, 1,4-dioxan-2-thion-5-yl, 1,4-dioxan-2-thion-6-yl, piperidin-2-on-1-yl, piperidin-2-on-3-yl, piperidin-2-on-4-yl, piperidin-2-on-5-yl, piperidin-2-on-6-yl, piperidin-2-thion-1-yl, piperidin-2-thion-3-yl, piperidin-2-thion-4-yl, piperidin-2-thion-5-yl, piperidin-2-thion-6-yl, hexahydropyrimidin-2-on-4-yl, hexahydropyrimidin-2-on-5-yl, hexahydropyrimidin-2-thion-4-yl, hexahydropyrimidin-2-thion-5-yl, piperazin-2-on-1-yl, piperazin-2-on-3-yl, piperazin-2-on-4-yl, piperazin-2-on-5-yl, piperazin-2-on-6-yl, piperazin-2-thion-1-yl, piperazin-2-thion-3-yl, piperazin-2-thion-4-yl, piperazin-2-thion-5-yl, piperazin-2-thion-6-yl, and the like.

Examples for 3-, 4-, 5- or 6-membered saturated, partially unsaturated or maximally unsaturated carbocyclic or heterocyclic ring containing 1, 2 or 3 heteroatoms or heteroatom groups selected from N, O, S, NO, SO, SO₂, C(O) and C(S) as ring members are the examples given above for 3-, 4-, 5- or 6-membered saturated, partially unsaturated or maximally unsaturated heterocyclic ring containing 1, 2, 3 or 4 heteroatoms or heteroatom groups selected from N, S, O, NO, SO and SO₂ and optionally also 1 or 2 groups C(═O) or C(═S) as ring members, and further following rings: cyclobutan-1-on-2-yl, cyclobutan-1-on-3-yl, 2-cyclobutan-1-thion-2-yl, cyclobutan-1-thion-3-yl, cyclopentan-1-on-2-yl, cyclopentan-1-on-3-yl, cyclopentan-1-thion-2-yl, cyclopentan-1-thion-3-yl, cyclohexan-1-on-2-yl, cyclohexan-1-on-3-yl, cyclohexan-1-on-4-yl, cyclohexan-1-thion-2-yl, cyclohexan-1-thion-3-yl, cyclohexan-1-thion-4-yl, cyclopent-2-en-1-on-2-yl, cyclopent-2-en-1-on-3-yl, cyclopent-2-en-1-on-4-yl, cyclopent-2-en-1-on-5-yl, cyclopent-2-en-1-thion-2-yl, cyclopent-2-en-1-thion-3-yl, cyclopent-2-en-1-thion-4-yl, cyclopent-2-en-1-thion-5-yl, cyclopent-3-en-1-on-2-yl, cyclopent-3-en-1-on-3-yl, cyclopent-3-en-1-on-4-yl, cyclopent-3-en-1-on-5-yl, cyclopent-3-en-1-thion-2-yl, cyclopent-3-en-1-thion-3-yl, cyclopent-3-en-1-thion-4-yl, cyclopent-3-en-1-thion-5-yl, cyclohex-2-en-1-on-2-yl, cyclohex-2-en-1-on-3-yl, cyclohex-2-en-1-on-4-yl, cyclohex-2-en-1-on-5-yl, cyclohex-2-en-1-on-6-yl, cyclohex-3-en-1-on-2-yl, cyclohex-3-en-1-on-3-yl, cyclohex-3-en-1-on-4-yl, cyclohex-3-en-1-on-5-yl, cyclohex-3-en-1-on-6-yl, cyclohex-2-en-1-thion-2-yl, cyclohex-2-en-1-thion-3-yl, cyclohex-2-en-1-thion-4-yl, cyclohex-2-en-1-thion-5-yl, cyclohex-2-en-1-thion-6-yl, cyclohex-3-en-1-thion-2-yl, cyclohex-3-en-1-thion-3-yl, cyclohex-3-en-1-thion-4-yl, cyclohex-3-en-1-thion-5-yl, cyclohex-3-en-1-thion-6-yl, cyclohexa-2,4-dien-1-on-2-yl, cyclohexa-2,4-dien-1-on-3-yl, cyclohexa-2,4-dien-1-on-4-yl, cyclohexa-2,4-dien-1-on-5-yl, cyclohexa-2,4-dien-1-on-6-yl, cyclohexa-2,5-dien-1-on-2-yl, cyclohexa-2,5-dien-1-on-3-yl, cyclohexa-2,5-dien-1-on-4-yl, cyclohexa-2,4-dien-1-thion-2-yl, cyclohexa-2,4-dien-1-thion-3-yl, cyclohexa-2,4-dien-1-thion-4-yl, cyclohexa-2,4-dien-1-thion-5-yl, cyclohexa-2,4-dien-1-thion-6-yl, cyclohexa-2,5-dien-1-thion-2-yl, cyclohexa-2,5-dien-1-thion-3-yl, cyclohexa-2,5-dien-1-thion-4-yl, and the like.

The remarks made below concerning preferred embodiments of the variables of the compounds of formula I, especially with respect to their substituents Q, Q-1, Q-2, Q-3, Q-4, Q-5, X, Y, L, A, B, G, J¹, J², Z, R^{A}, R^{B}, R^{y}, R^{a}, R^{b}, R^{c}, R^{c1}, R^{c2}, R^{c3}, R^{d}, R^{d1}, R^{d2}, R^{1a}, R^{1b}, R^{2a}, R^{2b}, R^{2c}, R^{2d}, R^{3a}, R^{3b}, R^{3c}, R^{3d}, R^{3e}, R⁴, R^{4a}, R⁵, R^{5a}, R⁶, R^{6a}, R⁷, R^{7a}, R⁸, R^{8a}, R⁹, R^{9a}, R¹⁰, R^{10a}, R¹¹, R^{11a}, R¹², R^{12a}, R¹³, R¹⁴, R¹⁵, R¹⁶, R¹⁷, R¹⁸, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, m, n and p, the features of the use and method according to the invention and of the composition of the invention are valid both on their own and, in particular, in every possible combination with each other.

In one embodiment, Q is a radical of formula Q-1.

In another embodiment, Q is a radical of formula Q-2.

In another embodiment, Q is a radical of formula Q-3.

In another embodiment, Q is a radical of formula Q-4.

In another embodiment, Q is a radical of formula Q-5.

Preferably, Q is a radical of formula Q-1 or Q-5. In particular, Q is a radical of formula Q-1.

In particular, X is CR^{3e}.

CR^{3e} is preferably selected from hydrogen, halogen and C₁-C₆-alkyl, and is in particular hydrogen.

Especially, X is CH.

L is preferably selected from a single bond, C₁-C₁₂-alkylene, C₁-C₁₂-haloalkylene, C₂-C₁₀-alkenylene and C₂-C₁₀-haloalkenylene, more preferably from a single bond, C₁-C₁₂-alkylene and C₁-C₁₂-haloalkylene, and in particular from a single bond and C₁-C₆-alkylene. Specifically, L is a single bond.

In a preferred embodiment A is selected from cyano, C₁-C₆-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₆-alkenyl which may be partially or fully halogenated and/or may be substituted by one or more radicals R²⁰; C₂-C₆-alkynyl which may be partially or fully halogenated and/or may be substituted by one or more radicals R²⁰; phenyl which may be substituted by 1, 2, 3, 4 or 5 radicals R²⁴; a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴; —C(═O)R²⁰; —C(═O)OR²¹; —C(═O)N(R²²)R²³; —C(═S)R²⁰; —C(═S)OR²¹; —C(═S)N(R²²)R²³; —N(R²²)R²³; —NR²²C(═O)R²⁰; —NR²²C(═O)OR²¹; —OR²¹; —SR²¹; —S(O)ₚR²¹; and —S(O)ₙN(R²²)R²³;

where R²⁰, R²¹, R²², R²³, R²⁴, n and p have one of the above general or, in particular, one of the below preferred meanings.

More preferably, A is selected from cyano, C₁-C₆-alkyl which may be partially or fully halogenated and/or may be substituted by one or more radicals R²⁰; C₃-C₈-cycloalkyl which may be partially or fully halogenated and/or may be substituted by one or more radicals R²⁰; phenyl which may be substituted by 1, 2, 3, 4 or 5 radicals R²⁴; a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴; —C(═O)R²⁰; —C(═O)OR²¹; —C(═O)N(R²²)R²³; —N(R²²)R²³, —NR²²C(═O)R²⁰; —NR²²C(═O)OR²¹; and —OR²¹;

where R²⁰, R²¹, R²², R²³ and R²⁴ have one of the above general or, in particular, one of the below preferred meanings.

In particular, A is selected from cyano, —NR²²C(═O)R²⁰ and —NR²²C(═O)OR²¹; where R²⁰, R²¹ and R²² have one of the above general or, in particular, one of the below preferred meanings.

In the above radicals —NR²²C(═O)R²⁰ and —NR²²C(═O)OR²¹ in the definition of A, preferably

R²⁰ is selected from hydrogen, C₁-C₆-alkyl and C₁-C₆-haloalkyl;
R²¹ is selected from hydrogen and C₁-C₆-alkyl; and
R²² is selected from hydrogen and C₁-C₄-alkyl, and is in particular hydrogen.

More preferably, in the above radicals —NR²²C(═O)R²⁰ and —NR²²C(═O)OR²¹ in the definition of A

R²⁰ is C₁-C₆-alkyl;
R²¹ is C₁-C₆-alkyl; and
R²² is hydrogen.

Specifically A is cyano.

In the above radicals Q-1, Q-2, Q-3 and Q-5, preferably J¹ is —C(R^{3a}R^{3b})— or —C(═O)—, and J² is —C(R^{3c}R^{3d})— or —C(═O)—, where R^{3a}, R^{3b}, R^{3c} and R^{3d} have one of the above general or, in particular, one of the below preferred meanings. More preferably, J¹ is —C(R^{3a}R^{3b})— and J² is —C(R^{3c}R^{3d})—.

Preferably, R^{3a}, R^{3b}, R^{3c} and R^{3d} are independently of each other selected from hydrogen, halogen and C₁-C₄-alkyl, more preferably from hydrogen and methyl, and are in particular hydrogen.

In particular, J¹ and J² are CH₂.

In a preferred embodiment R^{A} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰; C₁-C₁₀-alkoxy; C₁-C₁₀-haloalkoxy; —C(═O)R²⁰; —C(═O)OR²¹; —C(═O)N(R²²)R²³; phenyl which may be substituted by 1, 2, 3, 4 or 5 radicals R²⁴; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R²⁴,

where R²⁰, R²¹, R²², R²³ and R²⁴ have one of the above general or, in particular, one of the below preferred meanings.

More preferably, R^{A} is selected from the group consisting of hydrogen; cyano; C₁-C₆-alkyl; C₁-C₄-haloalkyl; C₁-C₄-alkoxy; C₁-C₄-haloalkoxy; and —C(═O)R²⁰; where R²⁰ has one of the above general or, in particular, one of the below preferred meanings. In an alternative preferred embodiment, R^{A} is hydrogen, C₁-C₄-alkyl or benzyl. Specifically R^{A} is hydrogen.

In a preferred embodiment Y is O or NR^{y}, where R^{y} has one of the above general or, in particular, one of the below preferred meanings. Preferably, R^{y} is hydrogen or C₁-C₄-alkyl. Thus, in particular, Y is O or NR^{y}, where R^{y} is hydrogen or C₁-C₄-alkyl. In particular, Y is O.

In a preferred embodiment R^{B} is selected from the group consisting of hydrogen; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰; —C(═O)R²⁰; —C(═O)OR²¹; —C(═O)N(R²²)R²³; —C(═S)R²⁰; —C(═S)OR²¹, —C(═S)N(R²²)R²³; —C(═NR²²)R²⁰; phenyl which may be substituted by 1, 2, 3, 4 or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;

where R²⁰, R²¹, R²², R²³ and R²⁴ have one of the above general or, in particular, one of the below preferred meanings.

More preferably R^{B} is selected from the group consisting of hydrogen, C₁-C₆-alkyl which may be partially or fully halogenated and/or may be substituted by one radical R²⁰; —C(═O)R²⁰; —C(═O)OR²¹; —C(═O)N(R²²)R²³; —C(═S)R²⁰; —C(═S)OR²¹, —C(═S)N(R²²)R²³; and phenyl which may be substituted by 1, 2 or 3 radicals R²⁴; where R²⁰, R²¹, R²², R²³ and R²⁴ have one of the above general or, in particular, one of the below preferred meanings.

In particular the combination of —Y—R^{B} is selected from
—O—R^{B1}, —NR^{y}—C(═O) R^{20a}, —NR^{y}—C(═O)N(R²²)R²³ and —NR^{y}—C(═S)N(R²²)R²³; where
R^{B1} is selected from hydrogen, C₁-C₆-alkyl, C₁-C₆-haloalkyl and a methyl group substituted by one radical R^{20b};
R^{y} is selected from hydrogen and C₁-C₆-alkyl, and is in particular hydrogen;
R^{20a} is selected from hydrogen, C₁-C₄-alkyl, C₁-C₄-haloalkyl, C₃-C₆-cycloalkyl and phenyl, where the phenyl ring may carry 1, 2 or 3 substituents selected from halogen, CN, C₁-C₄-alkyl, C₁-C₄-haloalkyl, C₁-C₄-alkoxy and C₁-C₄-haloalkoxy;
R^{20b} is selected from cyano, C₃-C₆-cycloalkyl and phenyl, where the phenyl ring may carry 1, 2 or 3 substituents selected from halogen, CN, C₁-C₄-alkyl, C₁-C₄-haloalkyl, C₁-C₄-alkoxy and C₁-C₄-haloalkoxy;
R²² is selected from hydrogen, C₁-C₄-alkyl and C₁-C₄-haloalkyl; and
R²³ is selected from hydrogen, CN, C₁-C₆-alkyl; C₁-C₆-haloalkyl; C₃-C₆-cycloalkyl; C₃-C₆-halocycloalkyl; C₃-C₆-cycloalkyl-C₁-C₄-alkyl; C₃-C₆-halocycloalkyl-C₁-C₄-alkyl; C₁-C₄-alkylcarbonyl, C₁-C₄-haloalkylcarbonyl, C₁-C₄-alkoxycarbonyl, C₁-C₄-haloalkoxycarbonyl, phenyl; benzyl and a 5- or 6-membered saturated, partially unsaturated or maximally unsaturated heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3 substituents selected from halogen, CN, C₁-C₄-alkyl, C₁-C₄-haloalkyl, C₁-C₄-alkoxy and C₁-C₄-haloalkoxy.

More preferably, the combination of —Y—R^{B} is selected from

—O—R^{B1}, —NR^{y}—C(═O)R^{20a}, —NR^{y}—C(═O)N(R²²)R²³ and —NR^{y}—C(═S)N(R²²)R²³; where
R^{B1} is selected from hydrogen, C₁-C₄-alkyl, C₁-C₄-haloalkyl and benzyl;
R^{y} is selected from hydrogen and C₁-C₄-alkyl, and is in particular hydrogen;
R^{20a} is C₁-C₄-alkyl;
R²² is hydrogen; and
R²³ is selected from hydrogen, C₁-C₆-alkyl and C₁-C₆-haloalkyl.

In particular, the combination of —Y—R^{B} is —O—R^{B1}, where

R^{B1} is selected from hydrogen, C₁-C₄-alkyl, C₁-C₄-haloalkyl and benzyl.

In a preferred embodiment G is C—R¹⁴, where R¹⁴ has one of the above general or, in particular, one of the below preferred meanings.

R¹⁴ is preferably selected from hydrogen, halogen, cyano, nitro, C₁-C₆-alkyl, C₁-C₆-haloalkyl, C₁-C₆-alkoxy, C₁-C₆-haloalkoxy, amino, C₁-C₆-alkylamino and di-(C₁-C₆-alkyl)-amino, more preferably from hydrogen, halogen, cyano, C₁-C₆-alkyl and C₁-C₆-haloalkyl, and is in particular hydrogen. Thus, in particular G is C—R¹⁴, where R¹⁴ is hydrogen.

Preferably, Z is selected from phenyl which may carry 1, 2 or 3 radicals R^{4a}; and pyridyl which may carry 1, 2 or 3 radicals R^{4a}; and is in particular phenyl which carries 1 or 2 radicals R^{4a}.

Preferably each R^{4a} is independently selected from halogen, C₁-C₄-alkyl, C₁-C₄-haloalkyl, C₁-C₄-alkoxy and C₁-C₄-haloalkoxy, and in particular from halogen, trifluoromethyl and trifluoromethoxy.

R^{2a}, R^{2b}, R^{2c} and R^{2d}, independently of each other, are preferably selected from hydrogen, halogen and C₁-C₄-alkyl, more preferably from hydrogen and methyl, and are in particular hydrogen.

Preferably, R^{1a} and R^{1b} are independently of each other selected from hydrogen, halogen and C₁-C₄-alkyl, more preferably from hydrogen and methyl, and are in particular hydrogen.

In a specific embodiment, the compounds I are compounds of formula I-A

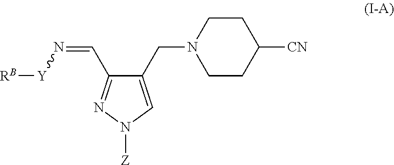

where Y, R^{B} and Z have one of the above general or, in particular, one of the above preferred meanings.

In one preferred embodiment in compounds I-A Z is 3,4-dichlorophenyl or 4-(trifluoromethyl)-phenyl; and

Y is O and R^{B} is hydrogen, methyl, ethyl, 2,2,2-trifluoroethyl or benzyl; or
Y is NH and R^{B} is acetyl, —C(═O)NHR²³ or —C(═S)NHR²³, where R²³ is methyl, ethyl or 2,2,2-trifluoroethyl.

In another preferred embodiment in compounds I-A Z is 3,4-dichlorophenyl, 4-(trifluoromethyl)-phenyl or 4-(trifluoromethoxy)-phenyl; Y is O; and R^{B} is hydrogen, C₁-C₆-alkyl, 2,2,2-trifluoroethyl or benzyl.

If not specified otherwise in a specific context, the below radicals have following preferred meanings.

R^{a} is preferably selected from hydrogen, C₁-C₆-alkyl, C₂-C₆-alkenyl, C₂-C₆-alkynyl, C₃-C₈-cycloalkyl, wherein the aliphatic and cycloaliphatic moieties in the 4 aforementioned radicals may be partially or fully halogenated and/or may carry 1 or 2 substituents selected from C₁-C₄-alkoxy and oxo; phenyl, benzyl and pyridyl, wherein the three last-mentioned radicals may carry one or more substituents selected from halogen, C₁-C₆-alkyl, C₁-C₆-haloalkyl, C₁-C₆-alkoxy, C₁-C₆-haloalkoxy and C₁-C₆-alkoxycarbonyl. More preferably, R^{a} is selected from hydrogen, C₁-C₆-alkyl, C₂-C₆-alkenyl, C₂-C₆-alkynyl, C₃-C₈-cycloalkyl, wherein the aliphatic and cycloaliphatic moieties in the 4 aforementioned radicals may be partially or fully halogenated and/or may carry 1 or 2 C₁-C₄-alkoxy substituents; phenyl, benzyl and pyridyl, wherein the three last-mentioned radicals may carry one or more substituents selected from halogen, C₁-C₆-alkyl, C₁-C₆-haloalkyl, C₁-C₆-alkoxy, C₁-C₆-haloalkoxy and C₁-C₆-alkoxycarbonyl. Even more preferably, R^{a} is selected from C₁-C₆-alkyl, C₁-C₆-haloalkyl, phenyl, benzyl and pyridyl, wherein the three last-mentioned radicals may carry one or more substituents selected from halogen, C₁-C₆-alkyl, C₁-C₆-haloalkyl, C₁-C₆-alkoxy, C₁-C₆-haloalkoxy and C₁-C₆-alkoxycarbonyl. In particular, R^{a} is selected from C₁-C₆-alkyl and C₁-C₆-haloalkyl.

R^{b} is preferably selected from hydrogen, C₁-C₆-alkyl and C₁-C₆-haloalkyl.

R^{c} is preferably selected from hydrogen, C₁-C₆-alkyl and C₁-C₆-haloalkyl.

R^{c1}, R^{c2} and R^{c3}, independently of each other, are preferably selected from hydrogen, C₁-C₆-alkyl, C₂-C₆-alkenyl, C₂-C₆-alkynyl, C₃-C₈-cycloalkyl, C₁-C₆-alkoxy, wherein the aliphatic and cycloaliphatic moieties in the 5 last-mentioned radicals may be partially or fully halogenated and/or may carry 1 or 2 substituents R¹⁸;

phenyl, benzyl, pyridyl and phenoxy, wherein the four last-mentioned radicals may carry one or more substituents selected from halogen, cyano, C₁-C₆-alkyl, C₁-C₆-haloalkyl, C₂-C₆-alkenyl, C₂-C₆-alkynyl, C₃-C₈-cycloalkyl, C₁-C₆-alkoxy, C₁-C₆-haloalkoxy, C₁-C₆-alkoxycarbonyl, C₁-C₆-alkylamino and di-(C₁-C₆-alkyl)amino.

In particular, they are selected from hydrogen, C₁-C₆-alkyl which may carry 1 radical R¹⁸, C₁-C₆-haloalkyl and C₁-C₆-alkoxy.

R^{d}, R^{d1} and R^{d2}, independently of each other and independently of each occurrence, are preferably selected from the group consisting of hydrogen, C₁-C₆-alkyl and C₁-C₆-haloalkyl.

Preferably each R⁴ is independently selected from halogen, cyano, nitro, C₁-C₆-alkyl, C₁-C₆-haloalkyl, C₁-C₆-alkoxy and C₁-C₆-haloalkoxy.

R⁵, R^{5a}, R⁶, R^{6a}, R⁷, R^{7a}, R⁸, R^{8a}, R⁹, R^{9a}, R¹⁰, R^{10a}, R¹¹, R^{11a}, R¹², R^{12a}, independently of each other, are preferably selected from hydrogen, C₁-C₆-alkyl, C₁-C₆-haloalkyl, C₃-C₆-cycloalkyl, C₃-C₆-halocycloalkyl and phenyl, and in particular from hydrogen, C₁-C₆-alkyl and C₁-C₆-haloalkyl.

Preferably each R¹³ is independently selected from halogen, cyano, nitro, C₁-C₆-alkyl, C₁-C₆-haloalkyl, C₁-C₆-alkoxy and C₁-C₆-haloalkoxy.

Preferably each R¹⁵ is independently selected from cyano, —OH, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-dialkylamino, C₁-C₆-alkylaminocarbonyl, C₁-C₆-dialkylaminocarbonyl and phenyl; and as a substituent on a cycloalkylene or halocycloalkylene moiety, R¹⁵ is additionally selected from C₁-C₆-alkyl, C₁-C₆-haloalkyl, C₂-C₆-alkenyl, C₂-C₆-haloalkenyl, C₂-C₆-alkynyl and C₂-C₆-haloalkynyl. In particular, each R¹⁵ is independently selected from cyano, C₃-C₆-cycloalkyl, C₃-C₆-halocycloalkyl, C₁-C₆-alkoxy, C₁-C₆-haloalkoxy and phenyl; and as a substituent on a cycloalkylene or halocycloalkylene moiety, R¹⁵ is additionally selected from C₁-C₄-alkyl and C₁-C₄-haloalkyl.

Preferably each R¹⁶ is independently selected from halogen, cyano, nitro, C₁-C₆-alkyl, C₁-C₆-haloalkyl, C₁-C₆-alkoxy and C₁-C₆-haloalkoxy.

Preferably, each R¹⁷ is independently selected from cyano, C₁-C₄-alkoxy and C₁-C₄-haloalkoxy; and as a substituent on a cycloalkylene or halocycloalkylene moiety, R¹⁷ is additionally selected from C₁-C₄-alkyl and C₁-C₄-haloalkyl.

Preferably, each R¹⁸ is independently selected from cyano, C₃-C₆-cycloalkyl, C₃-C₆-halocycloalkyl, C₁-C₆-alkoxy, C₁-C₆-haloalkoxy and phenyl; and as a substituent on a cycloalkylene or halocycloalkylene moiety, R¹⁸ is additionally selected from C₁-C₄-alkyl and C₁-C₄-haloalkyl.

In case R²⁰ is a substituent on an alkyl, alkenyl or alkynyl group, it is preferably selected from the group consisting of cyano, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, —C(═O)N(R²²)R²³, —C(═S)N(R²²)R²³, —C(═O)OR²¹, —C(═O)R³⁰, phenyl which may be substituted by 1, 2, 3, 4 or 5 radicals R²⁴, and a 5- or 6-membered heteroaromatic ring containing 1, 2 or 3 heteroatoms selected from N, O and S, as ring members, where the heteroaromatic ring may be substituted by one or more radicals R²⁴. In particular it selected from the group consisting of cyano, C₃-C₆-cycloalkyl, C₃-C₆-halocycloalkyl, C₁-C₄-alkoxy, C₁-C₄-haloalkoxy —C(═O)N(R²²)R²³, —C(═S)N(R²²)R²³, —C(═O)OR²¹, —C(═O)R³⁰, phenyl which may be substituted by 1, 2, 3, 4 or 5 radicals R²⁴, and a 5- or 6-membered heteroaromatic ring containing 1, 2 or 3 heteroatoms selected from N, O and S, as ring members, where the heteroaromatic ring may be substituted by one or more radicals R²⁴.

In case R²⁰ is a substituent on a cycloalkyl group, it preferably selected from the group consisting of cyano, C₁-C₆-alkyl, C₁-C₆-haloalkyl, C₁-C₆-alkoxy-C₁-C₆-alkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, —C(═O)N(R²²)R²³, —C(═S)N(R²²)R²³, —C(═O)OR²¹, —C(═O)R³⁰, phenyl which may be substituted by 1, 2, 3, 4 or 5 radicals R²⁴, and a 5- or 6-membered heteroaromatic ring containing 1, 2 or 3 heteroatoms selected from N, O and S, as ring members, where the heteroaromatic ring may be substituted by one or more radicals R²⁴. In particular it selected from the group consisting of cyano, C₁-C₆-alkyl, C₁-C₆-haloalkyl, C₁-C₄-alkoxy, C₁-C₄-haloalkoxy —C(═O)N(R²²)R²³, —C(═S)N(R²²)R²³, —C(═O)OR²¹, —C(═O)R³⁰, phenyl which may be substituted by 1, 2, 3, 4 or 5 radicals R²⁴, and a 5- or 6-membered heteroaromatic ring containing 1, 2 or 3 heteroatoms selected from N, O and S, as ring members, where the heteroaromatic ring may be substituted by one or more radicals R²⁴.

In groups —C(═O)R²⁰, —C(═S)R²⁰, —C(═NR²²)R²⁰ and —N(R²²)C(═O)R²⁰, R²⁰ is preferably selected from hydrogen, C₁-C₆-alkyl, C₁-C₆-haloalkyl, C₁-C₆-alkoxy-C₁-C₆-alkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₆-alkylamino, di(C₁-C₆-alkyl)amino, phenyl which may be substituted by 1, 2, 3, 4 or 5 radicals R²⁴, benzyl, and a 5- or 6-membered heteroaromatic ring containing 1, 2 or 3 heteroatoms selected from N, O and S, as ring members, where the heteroaromatic ring may be substituted by one or more radicals R²⁴. In particular it selected from the group consisting of hydrogen, C₁-C₆-alkyl, C₁-C₆-haloalkyl, phenyl and benzyl.

Preferably, each R²¹ is independently selected from the group consisting of hydrogen, C₁-C₆-alkyl, C₁-C₆-haloalkyl, C₃-C₈-cycloalkyl, C₃-C₆-halocycloalkyl, C₃-C₆-cycloalkyl-C₁-C₄-alkyl, phenyl which may be substituted by 1, 2, 3, 4 or 5 radicals R²⁴; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R²⁴, where R²⁴ has one of the meanings given above or in particular one of the preferred meanings given below. More preferably, each R²¹ is independently selected from the group consisting of hydrogen, C₁-C₆-alkyl, C₁-C₆-haloalkyl, phenyl which may be substituted by 1, 2, 3, 4 or 5 radicals R²⁴; and a 5- or 6-membered heteroaromatic ring containing 1, 2 or 3 heteroatoms selected from N, O and S, as ring members, where the heteroaromatic ring may be substituted by one or more radicals R²⁴; where R²⁴ has one of the meanings given above or in particular one of the preferred meanings given below.

R²² and R²³ are independently of each other and independently of each occurrence preferably selected from the group consisting of hydrogen, cyano, C₁-C₆-alkyl which may be partially or fully halogenated and/or may be substituted by one or more radicals R³⁰, C₂-C₆-alkenyl which may be partially or fully halogenated and/or may be substituted by one or more radicals R³⁰, C₂-C₆-alkynyl which may be partially or fully halogenated and/or may be substituted by one or more radicals R³⁰, C₃-C₈-cycloalkyl, C₃-C₈-halocycloalkyl, C₃-C₈-cycloalkyl-C₁-C₆-alkyl, S(O)ₘR³¹, S(O)ₙNR³⁵R³⁶, phenyl which may be substituted by 1, 2, 3, 4 or 5 radicals R²⁴, benzyl wherein the phenyl moiety may be substituted by 1, 2, 3, 4 or 5 radicals R²⁴, and a 5- or 6-membered heterocyclic ring containing 1, 2 or 3 heteroatoms or heteroatom groups selected from N, O, S, NO, SO and SO₂, as ring members, where the heterocyclic ring may be substituted by one or more radicals R²⁴; where R²⁴ has one of the meanings given above or in particular one of the preferred meanings given below; or

R²² and R²³ together form a group ═CR²⁷R²⁸; or
R²² and R²³, together with the nitrogen atom to which they are bound, form a 3-, 4-, 5-, 6- or 7-membered saturated, partially unsaturated or aromatic, preferably a saturated, heterocyclic ring which may additionally containing 1 or 2 further heteroatoms or heteroatom groups selected from N, O, S, NO, SO and SO₂, as ring members, where the heterocyclic ring may be substituted by one or more radicals R²⁴.

In the above preferred embodiment of R²² and R²³, R²⁷ is preferably hydrogen or methyl and R²⁸ is preferably C₁-C₆-alkoxy, C₁-C₆-haloalkoxy, —C(═O)R³⁰, —C(═O)OR³¹, or —C(═O)N(R³⁵)R³⁶.

In the above preferred embodiment of R²² and R²³, R²³, if it does not form together with R²² a group ═CR²⁷R²⁸ or together with R²² and the N atom to which they are bound a heterocyclic ring, is preferably selected from hydrogen, cyano, C₁-C₄-alkyl, C₁-C₄-haloalkyl, cyclopropyl, C₁-C₄-alkylcarbonyl, C₁-C₄-haloalkylcarbonyl, C₁-C₄-alkoxycarbonyl and C₁-C₄-haloalkoxycarbonyl and is more preferably hydrogen or C₁-C₄-alkyl.

In the above preferred embodiment of R²² and R²³, R²², if it does not form together with R⁹ a group ═CR²⁷R²⁸ or together with R²³ and the N atom to which they are bound a heterocyclic ring, is preferably selected from CN, C₁-C₆-alkyl; C₁-C₆-haloalkyl; C₁-C₄-alkyl which carries one radical R³⁰; C₂-C₆-alkenyl; C₂-C₆-haloalkenyl; C₂-C₄-alkenyl which is substituted by one radical R³⁰; C₃-C₆-cycloalkyl; C₃-C₆-halocycloalkyl; C₃-C₆-cycloalkyl-C₁-C₄-alkyl; C₃-C₆-halocycloalkyl-C₁-C₄-alkyl; —S(O)ₘR³³¹; —S(O)ₙN(R³⁵)R³⁶; phenyl; benzyl and a 5- or 6-membered saturated, partially unsaturated or aromatic heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3 substituents selected from halogen, CN, C₁-C₄-alkyl, C₁-C₄-haloalkyl, C₁-C₄-alkoxy and C₁-C₄-haloalkoxy.

If R²² and R²³, together with the nitrogen atom to which they are bound, form a 3-, 4-, 5-, 6- or 7-membered saturated, partially unsaturated or aromatic heterocyclic ring which may additionally containing 1 or 2 further heteroatoms or heteroatom groups selected from N, O, S, NO, SO and SO₂, as ring members, this is preferably a 3, 5 or 6-membered saturated heterocyclic ring which may additionally containing 1 further heteroatom or heteroatom group selected from N, O, S, NO, SO and SO₂, as ring member.

Specifically, R²² and R²³ are independently of each other and independently of each occurrence selected from the group consisting of hydrogen, C₁-C₆-alkyl, C₁-C₆-haloalkyl, C₂-C₆-alkynyl, C₃-C₈-cycloalkyl-C₁-C₆-alkyl, benzyl wherein the phenyl moiety may be substituted by 1, 2, 3, 4 or 5 radicals R²⁴, and a 5- or 6-membered heterocyclic ring containing 1, 2 or 3 heteroatoms or heteroatom groups selected from N, O, S, NO, SO and SO₂, as ring members, where the heterocyclic ring may be substituted by one or more radicals R²⁴. More specifically, R²³ is hydrogen or C₁-C₄-alkyl and R²² has one of the meanings specified above.

Preferably, each R²⁴ is independently selected from the group consisting of halogen, cyano, C₁-C₁₀-alkyl which may be partially or fully halogenated and/or may be substituted by one or more radicals R²⁰, —OR²¹, —N(R²²)R²³, C(═O)R²⁰, —C(═O)OR²¹, —C(═O)N(R²²)R²³, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;

or two radicals R²⁴ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, and —O(CH₂)O—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group,
where R²⁰, R²¹, R²² and R²³ have one of the general or in particular one of the preferred meanings given above.

More preferably, each R²⁴ is independently selected from the group consisting of halogen, cyano, C₁-C₁₀-alkyl which may be partially or fully halogenated and/or may be substituted by one or more radicals R²⁰, —OR²¹, —N(R²²)R²³, C(═O)R²⁰, —C(═O)OR²¹, —C(═O)N(R²²)R²³, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;

where R²⁰, R²¹, R²² and R²³ have one of the general or in particular one of the preferred meanings given above.

In particular, each R²⁴ is independently selected from the group consisting of halogen, cyano, C₁-C₄-alkyl, C₁-C₄-haloalkyl, C₁-C₄-alkoxy and C₁-C₄-haloalkoxy.

Preferably, R²⁵ and R²⁶ are, independently of each other and independently of each occurrence, selected from C₁-C₄-alkyl and are in particular methyl.

Preferably, R²⁷, R²⁸, R³⁷ and R³⁸ are, independently of each other and independently of each occurrence, selected from the group consisting of hydrogen, halogen, C₁-C₆-alkyl and C₁-C₆-haloalkyl. More preferably, R²⁷, R²⁸, R³⁷ and R³⁸ are, independently of each other and independently of each occurrence, selected from the group consisting of hydrogen, halogen and C₁-C₆-alkyl and in particular from the group consisting of hydrogen and halogen. Specifically, they are hydrogen.

Preferably, each R²⁹ is independently selected from the group consisting of hydrogen; C₁-C₁₀-alkyl which may be partially or fully halogenated and/or may be substituted by one or more, e.g. 1, 2, 3 or 4, preferably 1 or 2, more preferably 1, radicals R²⁰; —C(═O)R²⁰; —C(═O)OR²¹; —C(═O)N(R²²)R²³; —C(═S)R²⁰; —C(═S)OR²¹; —C(═S)N(R²²)R²³ and —C(═NR²²)R²⁰; where R²⁰, R²¹, R²² and R²³ have one of the general or in particular one of the preferred meanings given above.

More preferably, each R²⁹ is selected from the group consisting of hydrogen; C₁-C₆-alkyl which may be partially or fully halogenated and/or may be substituted by one or more, e.g. 1, 2, 3 or 4, preferably 1 or 2, more preferably 1, radicals R²⁰; —C(═O)R²⁰ and —C(═O)N(R²²)R²³; where R²⁰, R²² and R²³ have one of the general or in particular one of the preferred meanings given above. Preferably, in this case, R²⁰ as a C₁-C₆-alkyl substituent, is selected from CN, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio and a 5- or 6-membered hetaryl ring containing 1, 2 or 3 heteroatoms selected from N, O and S as ring members and being optionally substituted by 1, 2 or 3 radicals R²⁴. In this case, R²⁰ as a CO substituent, is preferably selected from C₁-C₆-alkyl, C₁-C₆-haloalkyl, C₁-C₆-alkoxy and C₁-C₆-haloalkoxy. In this case, R²² and R²³ are preferably selected from hydrogen and C₁-C₆-alkyl.

In particular, each R²⁹ is selected from the group consisting of hydrogen, C₁-C₄-alkyl, C₁-C₄-haloalkyl and —C(═O)R²⁰, and is specifically selected from the group consisting of hydrogen, C₁-C₄-alkyl and —C(═O)R²⁰, where R⁶ has one of the general or in particular one of the preferred meanings given above and is specifically C₁-C₄-alkyl.

In case R³⁰ is a substituent on an alkyl, alkenyl or alkynyl group, it is preferably selected from the group consisting of cyano, C₃-C₆-cycloalkyl, C₃-C₆-halocycloalkyl, —OR³¹, —C(═O)N(R³⁵)R³⁶, —C(═S)N(R³⁵)R³⁶, —C(═O)OR³¹, —C(═O)R³¹, phenyl which may be substituted by 1, 2, 3, 4 or 5 radicals R²⁴, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴;

where
R²⁴ is selected from halogen, cyano, C₁-C₄-alkyl, C₁-C₄-haloalkyl, C₁-C₄-alkoxy and C₁-C₄-haloalkoxy;
R³¹ is selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴; and
R³⁵ and R³⁶, independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴.

In case R³⁰ is a substituent on a cycloalkyl group, it is preferably selected from the group consisting of cyano, C₁-C₄-alkyl, C₁-C₄-haloalkyl, C₃-C₆-cycloalkyl, C₃-C₆-halocycloalkyl, —C(═O)N(R³⁵)R³⁶, —C(═S)N(R³⁵)R³⁶, —C(═O)OR³¹, —C(═O)R³¹, phenyl which may be substituted by 1, 2, 3, 4 or 5 radicals R²⁴, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴;

where
R²⁴ is selected from halogen, cyano, C₁-C₄-alkyl, C₁-C₄-haloalkyl, C₁-C₄-alkoxy and C₁-C₄-haloalkoxy;
R³¹ is selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴; and
R³⁵ and R³⁶, independently of each other and independently of each occurrence, are selected from hydrogen, C₁-C₄-alkyl, C₁-C₄-haloalkyl, phenyl, benzyl, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴.

In case R³⁰ is a substituent on a C(═O) group, it is preferably selected from the group consisting of hydrogen, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl which may be substituted by 1, 2, 3, 4 or 5 radicals R²⁴, benzyl which may be substituted by 1, 2, 3, 4 or 5 radicals R²⁴, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴; where R²⁴ is selected from halogen, cyano, C₁-C₄-alkyl, C₁-C₄-haloalkyl, C₁-C₄-alkoxy and C₁-C₄-haloalkoxy.

R³¹ is preferably selected from the group consisting of hydrogen, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₃-C₆-cycloalkyl-C₁-C₄-alkyl, phenyl which may be substituted by 1, 2, 3, 4 or 5 radicals R²⁴, benzyl which may be substituted by 1, 2, 3, 4 or 5 radicals R²⁴, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴; where R²⁴ is selected from halogen, cyano, C₁-C₄-alkyl, C₁-C₄-haloalkyl, C₁-C₄-alkoxy and C₁-C₄-haloalkoxy.

Preferably, R³² and R³³ are, independently of each other and independently of each occurrence, selected from the group consisting of C₁-C₆-alkyl, C₁-C₆-haloalkyl and phenyl which may be substituted by 1, 2, 3, 4, or 5 radicals R²⁴; where R²⁴ has one of the general or in particular one of the preferred meanings given above.

Preferably, each R³⁴ is independently selected from the group consisting of C₁-C₆-alkyl, C₁-C₆-haloalkyl, C₃-C₈-cycloalkyl, C₃-C₈-halocycloalkyl, phenyl and benzyl. More preferably, each R³⁴ is independently selected from the group consisting of C₁-C₆-alkyl, C₁-C₆-haloalkyl and phenyl and is in particular C₁-C₄-alkyl or C₁-C₃-haloalkyl.

R³⁵ and R³⁶, independently of each other and independently of each occurrence, are preferably selected from the group consisting of hydrogen, C₁-C₆-alkyl, C₁-C₆-haloalkyl, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R²⁴, benzyl in which the phenyl moiety may be substituted by 1, 2, 3, 4 or 5 radicals R²⁴, and a 5- or 6-membered heterocyclic ring containing 1, 2 or 3 heteroatoms or heteroatom groups selected from N, O, S, NO, SO and SO₂, as ring members, where the rings in the three last-mentioned radicals may be substituted by one or more radicals R²⁴; where R²⁴ is selected from halogen, cyano, C₁-C₄-alkyl, C₁-C₄-haloalkyl, C₁-C₄-alkoxy and C₁-C₄-haloalkoxy;

or R³⁵ and R³⁶, together with the nitrogen atom to which they are bound, may form a 5- or 6-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy.

Examples of preferred compounds are compounds of the following formulae I-1 to I-80, where the variables have one of the general or preferred meanings given above. Examples of preferred compounds are the individual compounds compiled in the tables 1 to 1600 below. Moreover, the meanings mentioned for the individual variables in the tables are per se, independently of the combination in which they are mentioned, a particularly preferred embodiment of the substituents in question.

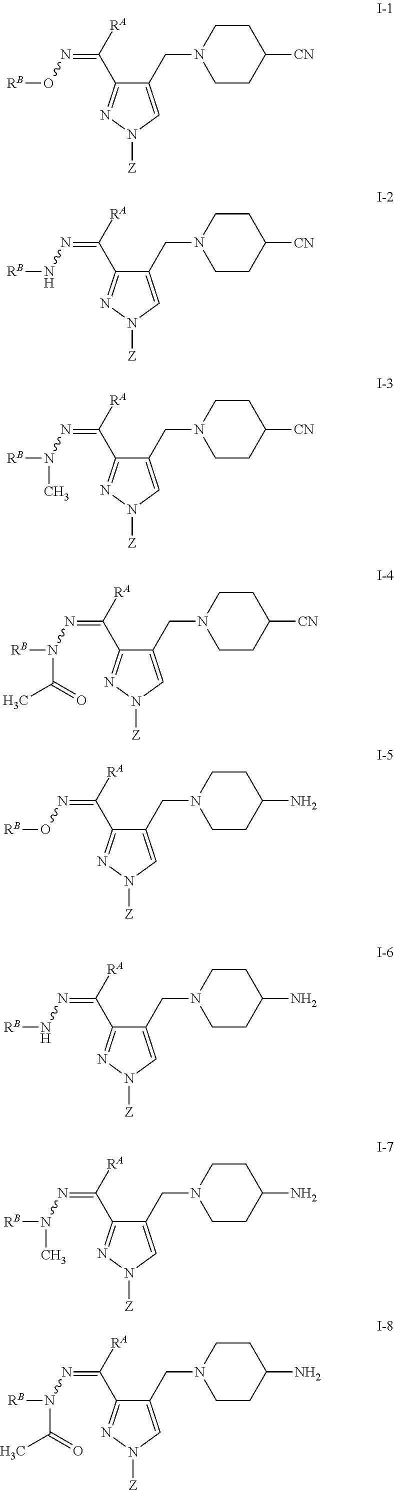

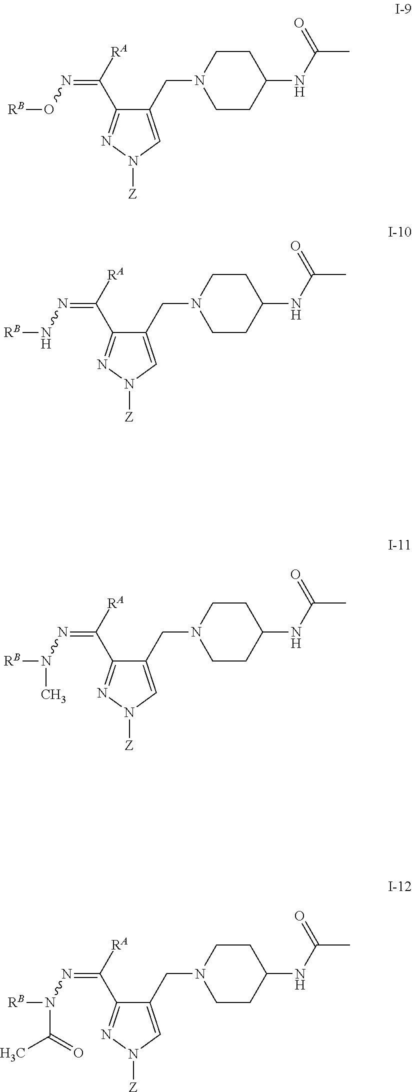

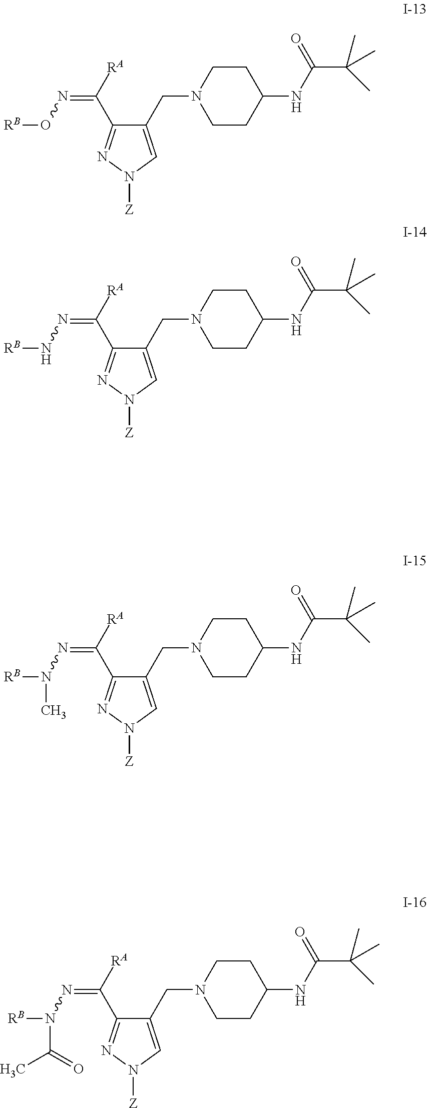

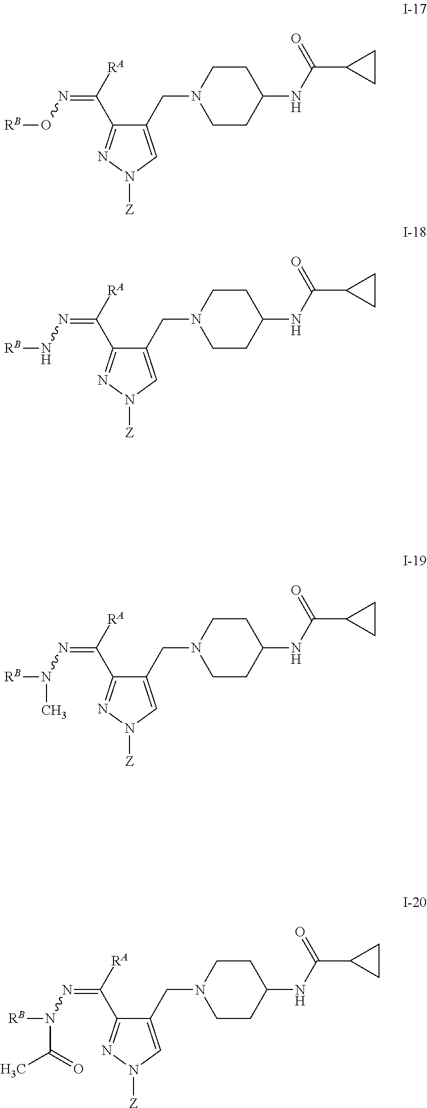

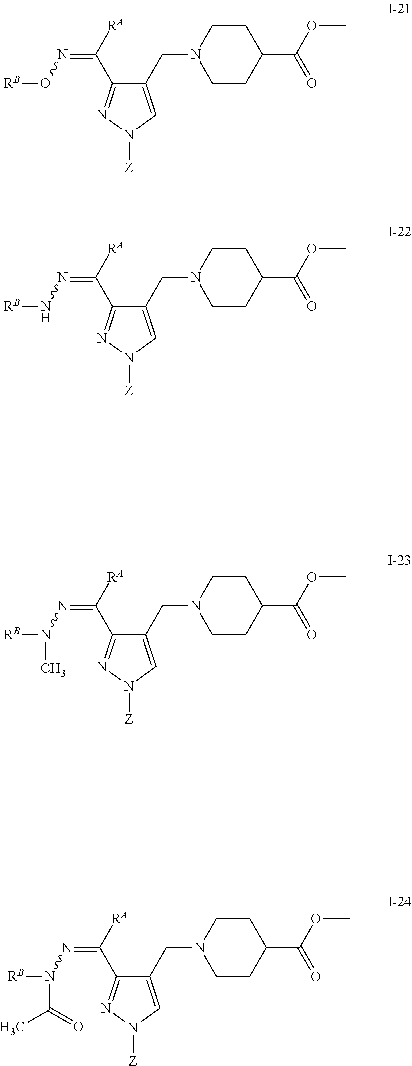

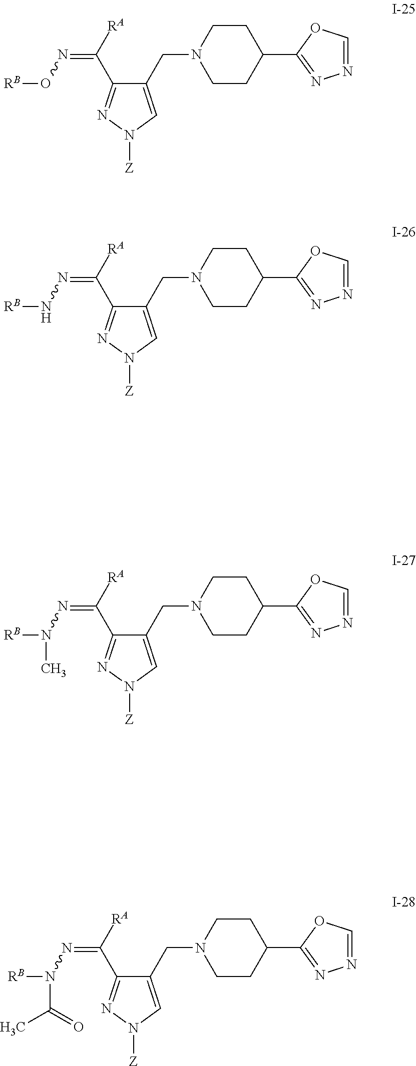

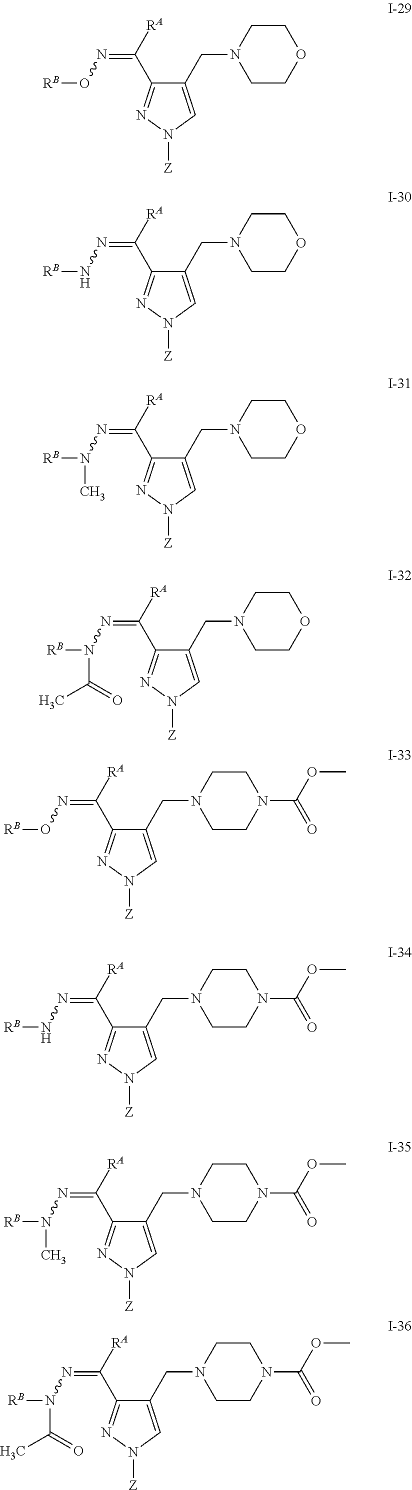

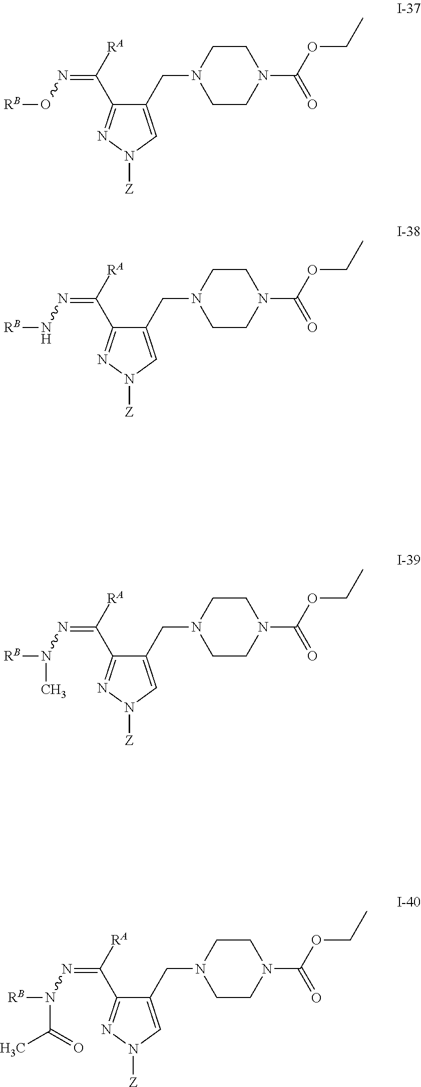

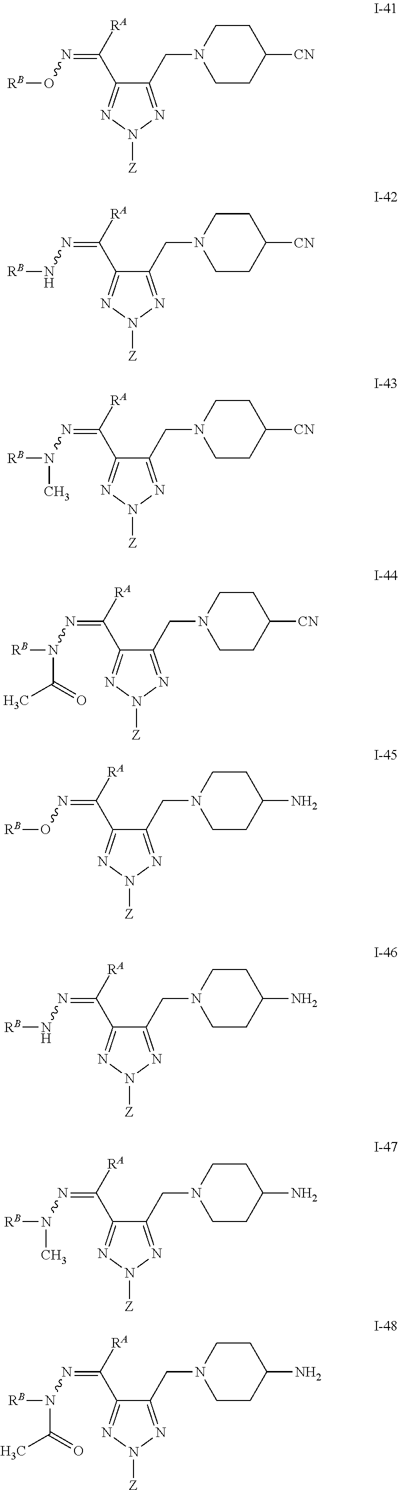

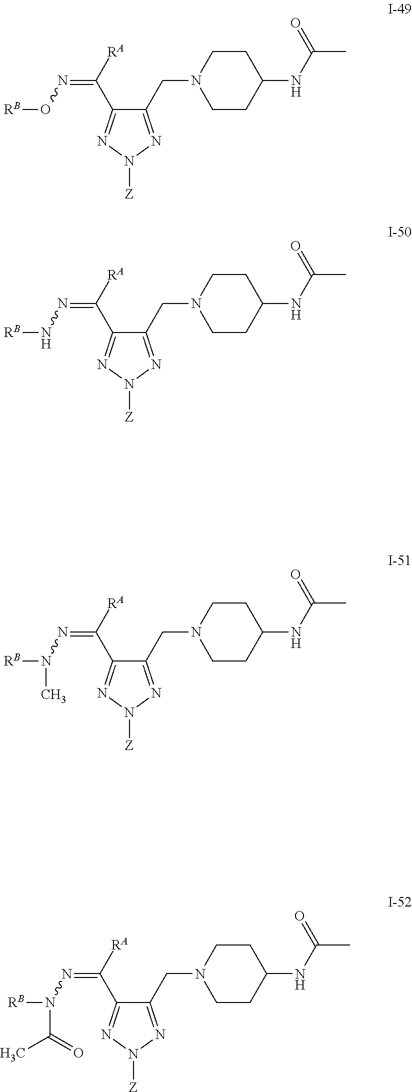

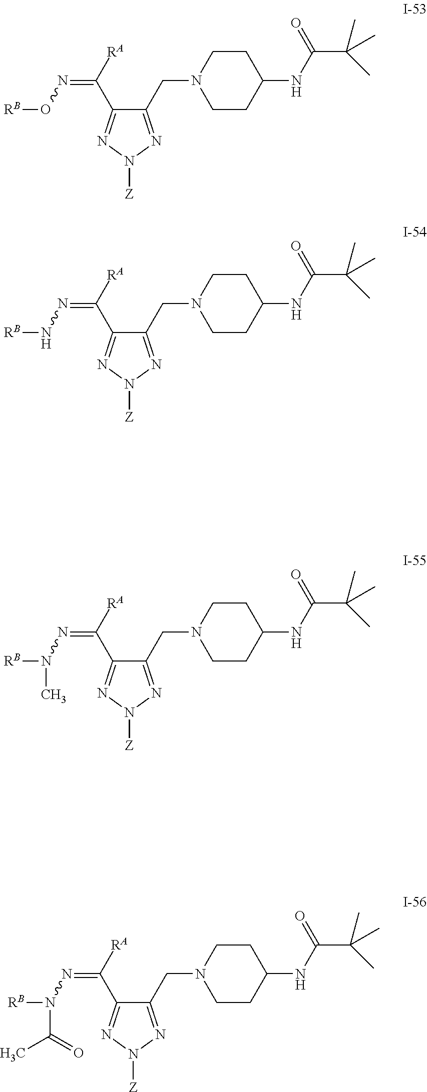

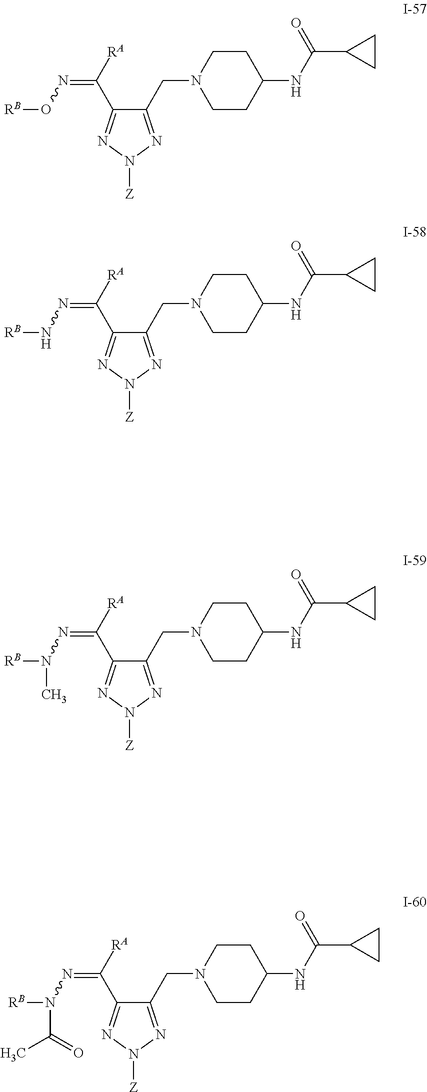

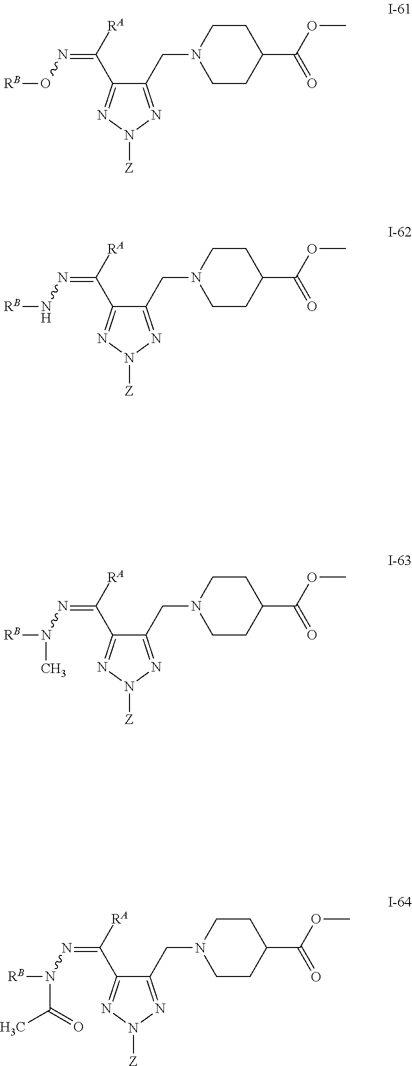

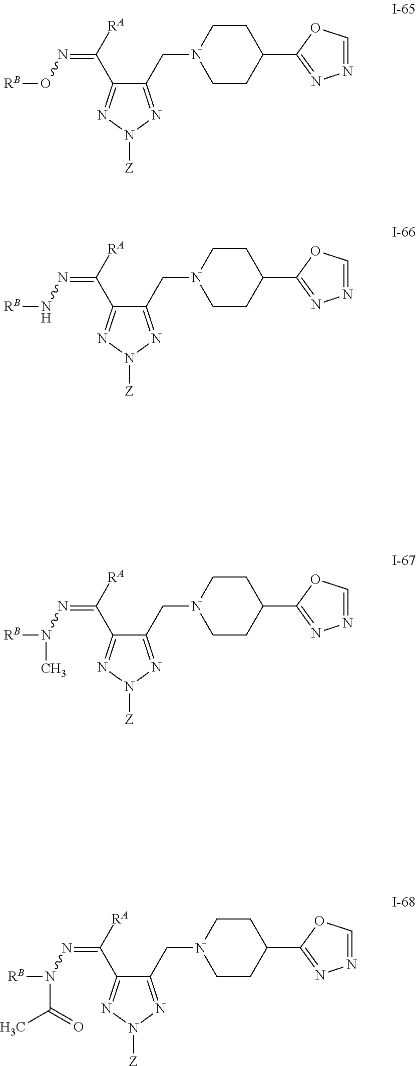

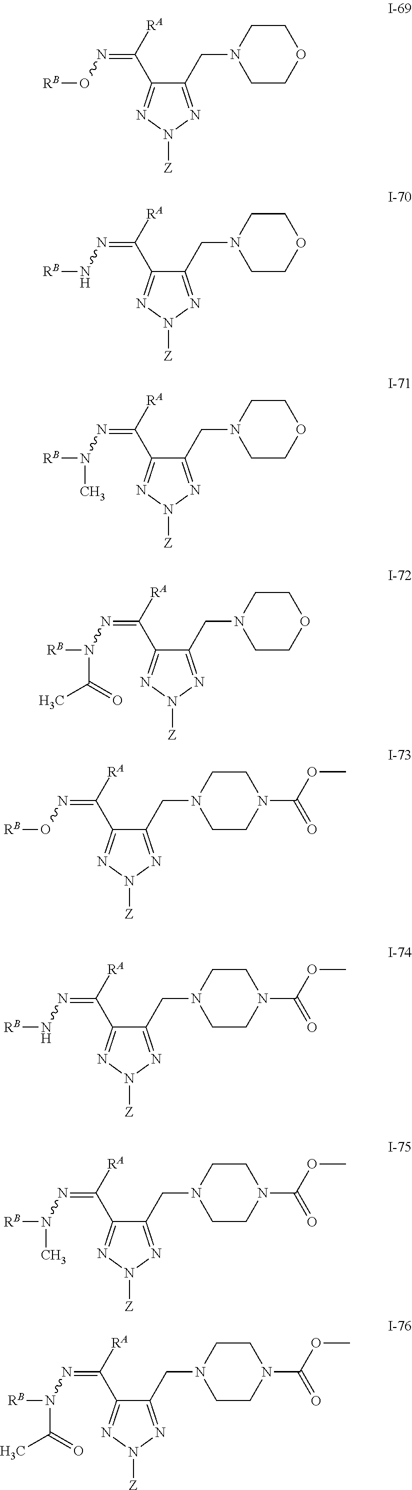

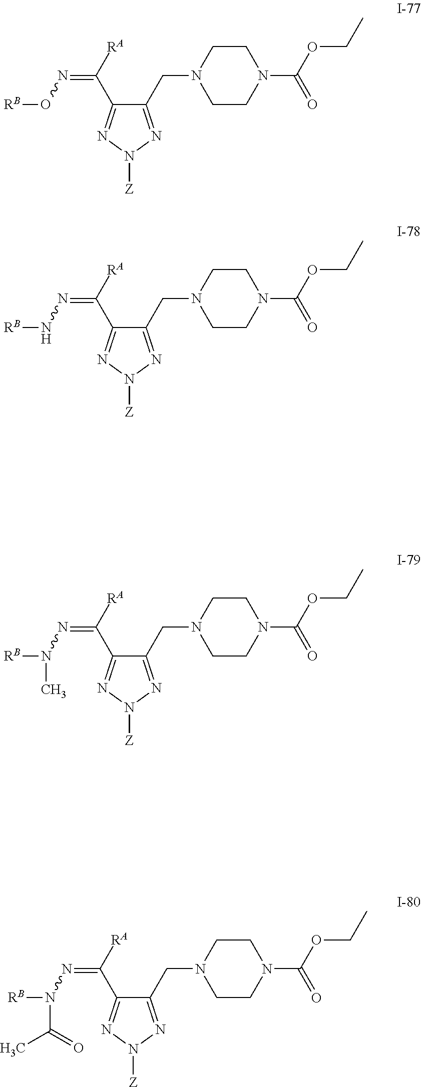

### Table 1

Compounds of the formula I-1 in which Z is 5-chloro-2-pyridinyl and the combination of R^{A} and R^{B} for a compound corresponds in each case to one row of Table A.

### Table 2

Compounds of the formula I-1 in which Z is 5-bromo-2-pyridinyl and the combination of R^{A} and R^{B} for a compound corresponds in each case to one row of Table A.

### Table 3

Compounds of the formula I-1 in which Z is 5-(trifluoromethyl)-2-pyridinyl and the combination of R^{A} and R^{B} for a compound corresponds in each case to one row of Table A.

### Table 4

Compounds of the formula I-1 in which Z is 6-chloro-3-pyridazinyl and the combination of R^{A} and R^{B} for a compound corresponds in each case to one row of Table A.

### Table 5

Compounds of the formula I-1 in which Z is 4-(trifluoromethyl)-2-pyrimidinyl and the combination of R^{A} and R^{B} for a compound corresponds in each case to one row of Table A.

### Table 6

Compounds of the formula I-1 in which Z is 4-chloro-5-fluoro-2-pyridinyl and the combination of R^{A} and R^{B} for a compound corresponds in each case to one row of Table A.

### Table 7

Compounds of the formula I-1 in which Z is 4,5-dichloro-2-pyridinyl and the combination of R^{A} and R^{B} for a compound corresponds in each case to one row of Table A.

### Table 8

Compounds of the formula I-1 in which Z is 5-(trifluoromethyl)-2-pyrimidinyl and the combination of R^{A} and R^{B} for a compound corresponds in each case to one row of Table A.

### Table 9

Compounds of the formula I-1 in which Z is 4-chloro-5-(trifluoromethyl)-2-pyrimidinyl and the combination of R^{A} and R^{B} for a compound corresponds in each case to one row of Table A.

### Table 10

Compounds of the formula I-1 in which Z is 4-chloro-4-fluoro-2-pyrimidinyl and the combination of R^{A} and R^{B} for a compound corresponds in each case to one row of Table A.

### Table 11

Compounds of the formula I-1 in which Z is 4-fluoro-5-(trifluoromethyl)-2-pyrimidinyl and the combination of R^{A} and R^{B} for a compound corresponds in each case to one row of Table A.

### Table 12

Compounds of the formula I-1 in which Z is 3-chlorophenyl and the combination of R^{A} and R^{B} for a compound corresponds in each case to one row of Table A.

### Table 13

Compounds of the formula I-1 in which Z is 4-chlorophenyl and the combination of R^{A} and R^{B} for a compound corresponds in each case to one row of Table A.

### Table 14

Compounds of the formula I-1 in which Z is 4-(trifluoromethyl)-phenyl and the combination of R^{A} and R^{B} for a compound corresponds in each case to one row of Table A.

### Table 15

Compounds of the formula I-1 in which Z is 3,4-dichlorophenyl and the combination of R^{A} and R^{B} for a compound corresponds in each case to one row of Table A.

### Table 16

Compounds of the formula I-1 in which Z is 3-(trifluoromethyl)-4-chlorophenyl and the combination of R^{A} and R^{B} for a compound corresponds in each case to one row of Table A.

### Table 17

Compounds of the formula I-1 in which Z is 3-(trifluoromethyl)-phenyl and the combination of R^{A} and R^{B} for a compound corresponds in each case to one row of Table A.

### Table 18

Compounds of the formula I-1 in which Z is 4-(trifluoromethoxy)-phenyl and the combination of R^{A} and R^{B} for a compound corresponds in each case to one row of Table A.

### Table 19

Compounds of the formula I-1 in which Z is 3-chloro-4-(trifluoromethyl)-phenyl and the combination of R^{A} and R^{B} for a compound corresponds in each case to one row of Table A.

### Table 20

Compounds of the formula I-1 in which Z is 3-(trifluoromethyl)-4-chlorophenyl and the combination of R^{A} and R^{B} for a compound corresponds in each case to one row of Table A.

### Tables 21 to 40

Compounds of the formula I-2 in which Z is as defined in any of tables 1 to 20 and the combination of R^{A} and R^{B} for a compound corresponds in each case to one row of Table A.

### Tables 41 to 60

Compounds of the formula I-3 in which Z is as defined in any of tables 1 to 20 and the combination of R^{A} and R^{B} for a compound corresponds in each case to one row of Table A.

### Tables 61 to 80

Compounds of the formula I-4 in which Z is as defined in any of tables 1 to 20 and the combination of R^{A} and R^{B} for a compound corresponds in each case to one row of Table A.

### Tables 81 to 100

Compounds of the formula I-5 in which Z is as defined in any of tables 1 to 20 and the combination of R^{A} and R^{B} for a compound corresponds in each case to one row of Table A.

### Tables 101 to 120

Compounds of the formula I-6 in which Z is as defined in any of tables 1 to 20 and the combination of R^{A} and R^{B} for a compound corresponds in each case to one row of Table A.

### Tables 121 to 140

Compounds of the formula I-7 in which Z is as defined in any of tables 1 to 20 and the combination of R^{A} and R^{B} for a compound corresponds in each case to one row of Table A.

### Tables 141 to 160

Compounds of the formula I-8 in which Z is as defined in any of tables 1 to 20 and the combination of R^{A} and R^{B} for a compound corresponds in each case to one row of Table A.

### Tables 161 to 180

Compounds of the formula I-9 in which Z is as defined in any of tables 1 to 20 and the combination of R^{A} and R^{B} for a compound corresponds in each case to one row of Table A.

### Tables 181 to 200

Compounds of the formula I-10 in which Z is as defined in any of tables 1 to 20 and the combination of R^{A} and R^{B} for a compound corresponds in each case to one row of Table A.

### Tables 201 to 220

Compounds of the formula I-11 in which Z is as defined in any of tables 1 to 20 and the combination of R^{A} and R^{B} for a compound corresponds in each case to one row of Table A.

### Tables 221 to 240

Compounds of the formula I-12 in which Z is as defined in any of tables 1 to 20 and the combination of R^{A} and R^{B} for a compound corresponds in each case to one row of Table A.

### Tables 241 to 260

Compounds of the formula I-13 in which Z is as defined in any of tables 1 to 20 and the combination of R^{A} and R^{B} for a compound corresponds in each case to one row of Table A.

### Tables 261 to 280

Compounds of the formula I-14 in which Z is as defined in any of tables 1 to 20 and the combination of R^{A} and R^{B} for a compound corresponds in each case to one row of Table A.

### Tables 281 to 300

Compounds of the formula I-15 in which Z is as defined in any of tables 1 to 20 and the combination of R^{A} and R^{B} for a compound corresponds in each case to one row of Table A.

### Tables 301 to 320

Compounds of the formula I-16 in which Z is as defined in any of tables 1 to 20 and the combination of R^{A} and R^{B} for a compound corresponds in each case to one row of Table A.

### Tables 321 to 340

Compounds of the formula I-17 in which Z is as defined in any of tables 1 to 20 and the combination of R^{A} and R^{B} for a compound corresponds in each case to one row of Table A.

### Tables 341 to 360

Compounds of the formula I-18 in which Z is as defined in any of tables 1 to 20 and the combination of R^{A} and R^{B} for a compound corresponds in each case to one row of Table A.

### Tables 361 to 380

Compounds of the formula I-19 in which Z is as defined in any of tables 1 to 20 and the combination of R^{A} and R^{B} for a compound corresponds in each case to one row of Table A.

### Tables 301 to 400

Compounds of the formula I-20 in which Z is as defined in any of tables 1 to 20 and the combination of R^{A} and R^{B} for a compound corresponds in each case to one row of Table A.

### Tables 401 to 420

Compounds of the formula I-21 in which Z is as defined in any of tables 1 to 20 and the combination of R^{A} and R^{B} for a compound corresponds in each case to one row of Table A.

### Tables 421 to 440

Compounds of the formula I-22 in which Z is as defined in any of tables 1 to 20 and the combination of R^{A} and R^{B} for a compound corresponds in each case to one row of Table A.

### Tables 441 to 460

Compounds of the formula I-23 in which Z is as defined in any of tables 1 to 20 and the combination of R^{A} and R^{B} for a compound corresponds in each case to one row of Table A.

### Tables 461 to 480

Compounds of the formula I-24 in which Z is as defined in any of tables 1 to 20 and the combination of R^{A} and R^{B} for a compound corresponds in each case to one row of Table A.

### Tables 481 to 500

Compounds of the formula I-25 in which Z is as defined in any of tables 1 to 20 and the combination of R^{A} and R^{B} for a compound corresponds in each case to one row of Table A.

### Tables 501 to 520

Compounds of the formula I-26 in which Z is as defined in any of tables 1 to 20 and the combination of R^{A} and R^{B} for a compound corresponds in each case to one row of Table A.

### Tables 521 to 540

Compounds of the formula I-27 in which Z is as defined in any of tables 1 to 20 and the combination of R^{A} and R^{B} for a compound corresponds in each case to one row of Table A.

### Tables 541 to 560

Compounds of the formula I-28 in which Z is as defined in any of tables 1 to 20 and the combination of R^{A} and R^{B} for a compound corresponds in each case to one row of Table A.

### Tables 561 to 580

Compounds of the formula I-29 in which Z is as defined in any of tables 1 to 20 and the combination of R^{A} and R^{B} for a compound corresponds in each case to one row of Table A.

### Tables 581 to 600

Compounds of the formula I-30 in which Z is as defined in any of tables 1 to 20 and the combination of R^{A} and R^{B} for a compound corresponds in each case to one row of Table A.

### Tables 601 to 620

Compounds of the formula I-31 in which Z is as defined in any of tables 1 to 20 and the combination of R^{A} and R^{B} for a compound corresponds in each case to one row of Table A.

### Tables 621 to 640

Compounds of the formula I-32 in which Z is as defined in any of tables 1 to 20 and the combination of R^{A} and R^{B} for a compound corresponds in each case to one row of Table A.

### Tables 641 to 660

Compounds of the formula I-33 in which Z is as defined in any of tables 1 to 20 and the combination of R^{A} and R^{B} for a compound corresponds in each case to one row of Table A.

### Tables 661 to 680

Compounds of the formula I-34 in which Z is as defined in any of tables 1 to 20 and the combination of R^{A} and R^{B} for a compound corresponds in each case to one row of Table A.

### Tables 681 to 700

Compounds of the formula I-35 in which Z is as defined in any of tables 1 to 20 and the combination of R^{A} and R^{B} for a compound corresponds in each case to one row of Table A.

### Tables 701 to 720

Compounds of the formula I-36 in which Z is as defined in any of tables 1 to 20 and the combination of R^{A} and R^{B} for a compound corresponds in each case to one row of Table A.

### Tables 721 to 740

Compounds of the formula I-37 in which Z is as defined in any of tables 1 to 20 and the combination of R^{A} and R^{B} for a compound corresponds in each case to one row of Table A.

### Tables 741 to 760

Compounds of the formula I-38 in which Z is as defined in any of tables 1 to 20 and the combination of R^{A} and R^{B} for a compound corresponds in each case to one row of Table A.

### Tables 761 to 780

Compounds of the formula I-39 in which Z is as defined in any of tables 1 to 20 and the combination of R^{A} and R^{B} for a compound corresponds in each case to one row of Table A.

### Tables 781 to 800

Compounds of the formula I-40 in which Z is as defined in any of tables 1 to 20 and the combination of R^{A} and R^{B} for a compound corresponds in each case to one row of Table A.

### Tables 801 to 820

Compounds of the formula I-41 in which Z is as defined in any of tables 1 to 20 and the combination of R^{A} and R^{B} for a compound corresponds in each case to one row of Table A.

### Tables 821 to 840

Compounds of the formula I-42 in which Z is as defined in any of tables 1 to 20 and the combination of R^{A} and R^{B} for a compound corresponds in each case to one row of Table A.

### Tables 841 to 860

Compounds of the formula I-43 in which Z is as defined in any of tables 1 to 20 and the combination of R^{A} and R^{B} for a compound corresponds in each case to one row of Table A.

### Tables 861 to 880

Compounds of the formula I-44 in which Z is as defined in any of tables 1 to 20 and the combination of R^{A} and R^{B} for a compound corresponds in each case to one row of Table A.

### Tables 881 to 900

Compounds of the formula I-45 in which Z is as defined in any of tables 1 to 20 and the combination of R^{A} and R^{B} for a compound corresponds in each case to one row of Table A.

### Tables 901 to 920

Compounds of the formula I-46 in which Z is as defined in any of tables 1 to 20 and the combination of R^{A} and R^{B} for a compound corresponds in each case to one row of Table A.

### Tables 921 to 940

Compounds of the formula I-47 in which Z is as defined in any of tables 1 to 20 and the combination of R^{A} and R^{B} for a compound corresponds in each case to one row of Table A.

### Tables 941 to 960

Compounds of the formula I-48 in which Z is as defined in any of tables 1 to 20 and the combination of R^{A} and R^{B} for a compound corresponds in each case to one row of Table A.

### Tables 961 to 980

Compounds of the formula I-49 in which Z is as defined in any of tables 1 to 20 and the combination of R^{A} and R^{B} for a compound corresponds in each case to one row of Table A.

### Tables 981 to 1000

Compounds of the formula I-50 in which Z is as defined in any of tables 1 to 20 and the combination of R^{A} and R^{B} for a compound corresponds in each case to one row of Table A.

### Tables 1001 to 1020

Compounds of the formula I-51 in which Z is as defined in any of tables 1 to 20 and the combination of R^{A} and R^{B} for a compound corresponds in each case to one row of Table A.

### Tables 1021 to 1040

Compounds of the formula I-52 in which Z is as defined in any of tables 1 to 20 and the combination of R^{A} and R^{B} for a compound corresponds in each case to one row of Table A.

### Tables 1041 to 1060

Compounds of the formula I-53 in which Z is as defined in any of tables 1 to 20 and the combination of R^{A} and R^{B} for a compound corresponds in each case to one row of Table A.

### Tables 1061 to 1080

Compounds of the formula I-54 in which Z is as defined in any of tables 1 to 20 and the combination of R^{A} and R^{B} for a compound corresponds in each case to one row of Table A.

### Tables 1081 to 1100

Compounds of the formula I-55 in which Z is as defined in any of tables 1 to 20 and the combination of R^{A} and R^{B} for a compound corresponds in each case to one row of Table A.

### Tables 1101 to 1120

Compounds of the formula I-56 in which Z is as defined in any of tables 1 to 20 and the combination of R^{A} and R^{B} for a compound corresponds in each case to one row of Table A.

### Tables 1121 to 1140

Compounds of the formula I-57 in which Z is as defined in any of tables 1 to 20 and the combination of R^{A} and R^{B} for a compound corresponds in each case to one row of Table A.

### Tables 1141 to 1160

Compounds of the formula I-58 in which Z is as defined in any of tables 1 to 20 and the combination of R^{A} and R^{B} for a compound corresponds in each case to one row of Table A.

### Tables 1161 to 1180

Compounds of the formula I-59 in which Z is as defined in any of tables 1 to 20 and the combination of R^{A} and R^{B} for a compound corresponds in each case to one row of Table A.

### Tables 1181 to 1200

Compounds of the formula I-60 in which Z is as defined in any of tables 1 to 20 and the combination of R^{A} and R^{B} for a compound corresponds in each case to one row of Table A.

### Tables 1201 to 1220

Compounds of the formula I-61 in which Z is as defined in any of tables 1 to 20 and the combination of R^{A} and R^{B} for a compound corresponds in each case to one row of Table A.

### Tables 1221 to 1240

Compounds of the formula I-62 in which Z is as defined in any of tables 1 to 20 and the combination of R^{A} and R^{B} for a compound corresponds in each case to one row of Table A.

### Tables 1241 to 1260

Compounds of the formula I-63 in which Z is as defined in any of tables 1 to 20 and the combination of R^{A} and R^{B} for a compound corresponds in each case to one row of Table A.

### Tables 1261 to 1280

Compounds of the formula I-64 in which Z is as defined in any of tables 1 to 20 and the combination of R^{A} and R^{B} for a compound corresponds in each case to one row of Table A.

### Tables 1281 to 1300

Compounds of the formula I-65 in which Z is as defined in any of tables 1 to 20 and the combination of R^{A} and R^{B} for a compound corresponds in each case to one row of Table A.

### Tables 1301 to 1320

Compounds of the formula I-66 in which Z is as defined in any of tables 1 to 20 and the combination of R^{A} and R^{B} for a compound corresponds in each case to one row of Table A.

### Tables 1321 to 1340

Compounds of the formula I-67 in which Z is as defined in any of tables 1 to 20 and the combination of R^{A} and R^{B} for a compound corresponds in each case to one row of Table A.

### Tables 1341 to 1360

Compounds of the formula I-68 in which Z is as defined in any of tables 1 to 20 and the combination of R^{A} and R^{B} for a compound corresponds in each case to one row of Table A.

### Tables 1361 to 1380

Compounds of the formula I-69 in which Z is as defined in any of tables 1 to 20 and the combination of R^{A} and R^{B} for a compound corresponds in each case to one row of Table A.

### Tables 1381 to 1400

Compounds of the formula I-70 in which Z is as defined in any of tables 1 to 20 and the combination of R^{A} and R^{B} for a compound corresponds in each case to one row of Table A.

### Tables 1401 to 1420

Compounds of the formula I-71 in which Z is as defined in any of tables 1 to 20 and the combination of R^{A} and R^{B} for a compound corresponds in each case to one row of Table A.

### Tables 1421 to 1440

Compounds of the formula I-72 in which Z is as defined in any of tables 1 to 20 and the combination of R^{A} and R^{B} for a compound corresponds in each case to one row of Table A.

### Tables 1441 to 1460

Compounds of the formula I-73 in which Z is as defined in any of tables 1 to 20 and the combination of R^{A} and R^{B} for a compound corresponds in each case to one row of Table A.

### Tables 1461 to 1480

Compounds of the formula I-74 in which Z is as defined in any of tables 1 to 20 and the combination of R^{A} and R^{B} for a compound corresponds in each case to one row of Table A.

### Tables 1481 to 1500

Compounds of the formula I-75 in which Z is as defined in any of tables 1 to 20 and the combination of R^{A} and R^{B} for a compound corresponds in each case to one row of Table A.

### Tables 1501 to 1520

Compounds of the formula I-76 in which Z is as defined in any of tables 1 to 20 and the combination of R^{A} and R^{B} for a compound corresponds in each case to one row of Table A.

### Tables 1521 to 1540

Compounds of the formula I-77 in which Z is as defined in any of tables 1 to 20 and the combination of R^{A} and R^{B} for a compound corresponds in each case to one row of Table A.

### Tables 1541 to 1560

Compounds of the formula I-78 in which Z is as defined in any of tables 1 to 20 and the combination of R^{A} and R^{B} for a compound corresponds in each case to one row of Table A.

### Tables 1561 to 1580

Compounds of the formula I-79 in which Z is as defined in any of tables 1 to 20 and the combination of R^{A} and R^{B} for a compound corresponds in each case to one row of Table A.

### Tables 1581 to 1600

Compounds of the formula I-80 in which Z is as defined in any of tables 1 to 20 and the combination of R^{A} and R^{B} for a compound corresponds in each case to one row of Table A.

TABLE A

| No. | R^{A} | R^{B} |
|---|---|---|
| 1. | H | H |
| 2. | H | CH₃ |
| 3. | H | CH₂CH₃ |
| 4. | H | CH₂CH₂CH₃ |
| 5. | H | CH(CH₃)₂ |
| 6. | H | CH₂CH₂CH₂CH₃ |
| 7. | H | CH₂CH(CH₃)₂ |
| 8. | H | CHF₂ |
| 9. | H | CF₃ |
| 10. | H | CH₂CF₃ |
| 11. | H | CH₂C₆H₅ |
| 12. | H | C₆H₅ |
| 13. | H | 4-Cl—C₆H₄ |
| 14. | H | 2,4-Cl₂—C₆H₃ |
| 15. | H | C(═O)H |
| 16. | H | C(═O)CH₃ |
| 17. | H | C(═O)CH₂CH₃ |
| 18. | H | C(═O)CH₂CH₂CH₃ |
| 19. | H | C(═O)CHF₂ |
| 20. | H | C(═O)CF₃ |
| 21. | H | C(═O)CH₂CHF₂ |
| 22. | H | C(═O)CH₂CF₃ |
| 23. | H | C(═O)C₆H₅ |
| 24. | H | C(═O)CH₂C₆H₅ |
| 25. | H | C(═O)NH₂ |
| 26. | H | C(═O)NHCH₃ |
| 27. | H | C(═O)NHCH₂CH₃ |
| 28. | H | C(═O)NHCH₂CH₂CH₃ |
| 29. | H | C(═O)NHCHF₂ |
| 30. | H | C(═O)NHCF₃ |
| 31. | H | C(═O)NHCH₂CHF₂ |
| 32. | H | C(═O)NHCH₂CF₃ |
| 33. | CH₃ | H |
| 34. | CH₃ | CH₃ |
| 35. | CH₃ | CH₂CH₃ |
| 36. | CH₃ | CH₂CH₂CH₃ |
| 37. | CH₃ | CH(CH₃)₂ |
| 38. | CH₃ | CH₂CH₂CH₂CH₃ |
| 39. | CH₃ | CH₂CH(CH₃)₂ |
| 40. | CH₃ | CHF₂ |
| 41. | CH₃ | CF₃ |
| 42. | CH₃ | CH₂CF₃ |
| 43. | CH₃ | CH₂C₆H₅ |
| 44. | CH₃ | C₆H₅ |
| 45. | CH₃ | 4-Cl—C₆H₄ |
| 46. | CH₃ | 2,4-Cl₂—C₆H₃ |
| 47. | CH₃ | C(═O)H |
| 48. | CH₃ | C(═O)CH₃ |
| 49. | CH₃ | C(═O)CH₂CH₃ |
| 50. | CH₃ | C(═O)CH₂CH₂CH₃ |
| 51. | CH₃ | C(═O)CHF₂ |
| 52. | CH₃ | C(═O)CF₃ |
| 53. | CH₃ | C(═O)CH₂CHF₂ |
| 54. | CH₃ | C(═O)CH₂CF₃ |
| 55. | CH₃ | C(═O)C₆H₅ |
| 56. | CH₃ | C(═O)CH₂C₆H₅ |
| 57. | CH₃ | C(═O)NH₂ |
| 58. | CH₃ | C(═O)NHCH₃ |
| 59. | CH₃ | C(═O)NHCH₂CH₃ |
| 60. | CH₃ | C(═O)NHCH₂CH₂CH₃ |
| 61. | CH₃ | C(═O)NHCHF₂ |
| 62. | CH₃ | C(═O)NHCF₃ |
| 63. | CH₃ | C(═O)NHCH₂CHF₂ |
| 64. | CH₃ | C(═O)NHCH₂CF₃ |
| 65. | CH₂CH₃ | H |
| 66. | CH₂CH₃ | CH₃ |
| 67. | CH₂CH₃ | CH₂CH₃ |
| 68. | CH₂CH₃ | CH₂CH₂CH₃ |
| 69. | CH₂CH₃ | CH(CH₃)₂ |
| 70. | CH₂CH₃ | CH₂CH₂CH₂CH₃ |
| 71. | CH₂CH₃ | CH₂CH(CH₃)₂ |
| 72. | CH₂CH₃ | CHF₂ |
| 73. | CH₂CH₃ | CF₃ |
| 74. | CH₂CH₃ | CH₂CF₃ |
| 75. | CH₂CH₃ | CH₂C₆H₅ |
| 76. | CH₂CH₃ | C₆H₅ |
| 77. | CH₂CH₃ | 4-Cl—C₆H₄ |
| 78. | CH₂CH₃ | 2,4-Cl₂—C₆H₃ |
| 79. | CH₂CH₃ | C(═O)H |
| 80. | CH₂CH₃ | C(═O)CH₃ |
| 81. | CH₂CH₃ | C(═O)CH₂CH₃ |
| 82. | CH₂CH₃ | C(═O)CH₂CH₂CH₃ |
| 83. | CH₂CH₃ | C(═O)CHF₂ |
| 84. | CH₂CH₃ | C(═O)CF₃ |
| 85. | CH₂CH₃ | C(═O)CH₂CHF₂ |
| 86. | CH₂CH₃ | C(═O)CH₂CF₃ |
| 87. | CH₂CH₃ | C(═O)C₆H₅ |
| 88. | CH₂CH₃ | C(═O)CH₂C₆H₅ |
| 89. | CH₂CH₃ | C(═O)NH₂ |
| 90. | CH₂CH₃ | C(═O)NHCH₃ |
| 91. | CH₂CH₃ | C(═O)NHCH₂CH₃ |
| 92. | CH₂CH₃ | C(═O)NHCH₂CH₂CH₃ |
| 93. | CH₂CH₃ | C(═O)NHCHF₂ |
| 94. | CH₂CH₃ | C(═O)NHCF₃ |
| 95. | CH₂CH₃ | C(═O)NHCH₂CHF₂ |
| 96. | CH₂CH₃ | C(═O)NHCH₂CF₃ |
| 97. | C(═O)CH₃ | H |
| 98. | C(═O)CH₃ | CH₃ |
| 99. | C(═O)CH₃ | CH₂CH₃ |
| 100. | C(═O)CH₃ | CH₂CH₂CH₃ |
| 101. | C(═O)CH₃ | CH(CH₃)₂ |
| 102. | C(═O)CH₃ | CH₂CH₂CH₂CH₃ |
| 103. | C(═O)CH₃ | CH₂CH(CH₃)₂ |
| 104. | C(═O)CH₃ | CHF₂ |
| 105. | C(═O)CH₃ | CF₃ |
| 106. | C(═O)CH₃ | CH₂CF₃ |
| 107. | C(═O)CH₃ | CH₂C₆H₅ |
| 108. | C(═O)CH₃ | C₆H₅ |
| 109. | C(═O)CH₃ | 4-Cl—C₆H₄ |
| 110. | C(═O)CH₃ | 2,4-Cl₂—C₆H₃ |
| 111. | C(═O)CH₃ | C(═O)H |
| 112. | C(═O)CH₃ | C(═O)CH₃ |
| 113. | C(═O)CH₃ | C(═O)CH₂CH₃ |
| 114. | C(═O)CH₃ | C(═O)CH₂CH₂CH₃ |
| 115. | C(═O)CH₃ | C(═O)CHF₂ |
| 116. | C(═O)CH₃ | C(═O)CF₃ |
| 117. | C(═O)CH₃ | C(═O)CH₂CHF₂ |
| 118. | C(═O)CH₃ | C(═O)CH₂CF₃ |
| 119. | C(═O)CH₃ | C(═O)C₆H₅ |
| 120. | C(═O)CH₃ | C(═O)CH₂C₆H₅ |
| 121. | C(═O)CH₃ | C(═O)NH₂ |
| 122. | C(═O)CH₃ | C(═O)NHCH₃ |
| 123. | C(═O)CH₃ | C(═O)NHCH₂CH₃ |
| 124. | C(═O)CH₃ | C(═O)NHCH₂CH₂CH₃ |
| 125. | C(═O)CH₃ | C(═O)NHCHF₂ |
| 126. | C(═O)CH₃ | C(═O)NHCF₃ |
| 127. | C(═O)CH₃ | C(═O)NHCH₂CHF₂ |
| 128. | C(═O)CH₃ | C(═O)NHCH₂CF₃ |
| 129. | C(═O)CH₂CH₃ | H |
| 130. | C(═O)CH₂CH₃ | CH₃ |
| 131. | C(═O)CH₂CH₃ | CH₂CH₃ |
| 132. | C(═O)CH₂CH₃ | CH₂CH₂CH₃ |
| 133. | C(═O)CH₂CH₃ | CH(CH₃)₂ |
| 134. | C(═O)CH₂CH₃ | CH₂CH₂CH₂CH₃ |
| 135. | C(═O)CH₂CH₃ | CH₂CH(CH₃)₂ |
| 136. | C(═O)CH₂CH₃ | CHF₂ |
| 137. | C(═O)CH₂CH₃ | CF₃ |
| 138. | C(═O)CH₂CH₃ | CH₂CF₃ |
| 139. | C(═O)CH₂CH₃ | CH₂C₆H₅ |
| 140. | C(═O)CH₂CH₃ | C₆H₅ |
| 141. | C(═O)CH₂CH₃ | 4-Cl—C₆H₄ |
| 142. | C(═O)CH₂CH₃ | 2,4-Cl₂—C₆H₃ |
| 143. | C(═O)CH₂CH₃ | C(═O)H |
| 144. | C(═O)CH₂CH₃ | C(═O)CH₃ |
| 145. | C(═O)CH₂CH₃ | C(═O)CH₂CH₃ |
| 146. | C(═O)CH₂CH₃ | C(═O)CH₂CH₂CH₃ |
| 147. | C(═O)CH₂CH₃ | C(═O)CHF₂ |
| 148. | C(═O)CH₂CH₃ | C(═O)CF₃ |
| 149. | C(═O)CH₂CH₃ | C(═O)CH₂CHF₂ |
| 150. | C(═O)CH₂CH₃ | C(═O)CH₂CF₃ |
| 151. | C(═O)CH₂CH₃ | C(═O)C₆H₅ |
| 152. | C(═O)CH₂CH₃ | C(═O)CH₂C₆H₅ |
| 153. | C(═O)CH₂CH₃ | C(═O)NH₂ |
| 154. | C(═O)CH₂CH₃ | C(═O)NHCH₃ |
| 155. | C(═O)CH₂CH₃ | C(═O)NHCH₂CH₃ |
| 156. | C(═O)CH₂CH₃ | C(═O)NHCH₂CH₂CH₃ |
| 157. | C(═O)CH₂CH₃ | C(═O)NHCHF₂ |
| 158. | C(═O)CH₂CH₃ | C(═O)NHCF₃ |
| 159. | C(═O)CH₂CH₃ | C(═O)NHCH₂CHF₂ |
| 160. | C(═O)CH₂CH₃ | C(═O)NHCH₂CF₃ |
| 161. | C(═O)OH | H |
| 162. | C(═O)OH | CH₃ |
| 163. | C(═O)OH | CH₂CH₃ |
| 164. | C(═O)OH | CH₂CH₂CH₃ |
| 165. | C(═O)OH | CH(CH₃)₂ |
| 166. | C(═O)OH | CH₂CH₂CH₂CH₃ |
| 167. | C(═O)OH | CH₂CH(CH₃)₂ |
| 168. | C(═O)OH | CHF₂ |
| 169. | C(═O)OH | CF₃ |
| 170. | C(═O)OH | CH₂CF₃ |
| 171. | C(═O)OH | CH₂C₆H₅ |
| 172. | C(═O)OH | C₆H₅ |
| 173. | C(═O)OH | 4-Cl—C₆H₄ |
| 174. | C(═O)OH | 2,4-Cl₂—C₆H₃ |
| 175. | C(═O)OH | C(═O)H |
| 176. | C(═O)OH | C(═O)CH₃ |
| 177. | C(═O)OH | C(═O)CH₂CH₃ |
| 178. | C(═O)OH | C(═O)CH₂CH₂CH₃ |
| 179. | C(═O)OH | C(═O)CHF₂ |
| 180. | C(═O)OH | C(═O)CF₃ |
| 181. | C(═O)OH | C(═O)CH₂CHF₂ |
| 182. | C(═O)OH | C(═O)CH₂CF₃ |
| 183. | C(═O)OH | C(═O)C₆H₅ |
| 184. | C(═O)OH | C(═O)CH₂C₆H₅ |
| 185. | C(═O)OH | C(═O)NH₂ |
| 186. | C(═O)OH | C(═O)NHCH₃ |
| 187. | C(═O)OH | C(═O)NHCH₂CH₃ |
| 188. | C(═O)OH | C(═O)NHCH₂CH₂CH₃ |
| 189. | C(═O)OH | C(═O)NHCHF₂ |
| 190. | C(═O)OH | C(═O)NHCF₃ |
| 191. | C(═O)OH | C(═O)NHCH₂CHF₂ |
| 192. | C(═O)OH | C(═O)NHCH₂CF₃ |
| 193. | C(═O)OCH₃ | H |
| 194. | C(═O)OCH₃ | CH₃ |
| 195. | C(═O)OCH₃ | CH₂CH₃ |
| 196. | C(═O)OCH₃ | CH₂CH₂CH₃ |
| 197. | C(═O)OCH₃ | CH(CH₃)₂ |
| 198. | C(═O)OCH₃ | CH₂CH₂CH₂CH₃ |
| 199. | C(═O)OCH₃ | CH₂CH(CH₃)₂ |
| 200. | C(═O)OCH₃ | CHF₂ |
| 201. | C(═O)OCH₃ | CF₃ |
| 202. | C(═O)OCH₃ | CH₂CF₃ |
| 203. | C(═O)OCH₃ | CH₂C₆H₅ |
| 204. | C(═O)OCH₃ | C₆H₅ |
| 205. | C(═O)OCH₃ | 4-Cl—C₆H₄ |
| 206. | C(═O)OCH₃ | 2,4-Cl₂—C₆H₃ |
| 207. | C(═O)OCH₃ | C(═O)H |
| 208. | C(═O)OCH₃ | C(═O)CH₃ |
| 209. | C(═O)OCH₃ | C(═O)CH₂CH₃ |
| 210. | C(═O)OCH₃ | C(═O)CH₂CH₂CH₃ |
| 211. | C(═O)OCH₃ | C(═O)CHF₂ |
| 212. | C(═O)OCH₃ | C(═O)CF₃ |
| 213. | C(═O)OCH₃ | C(═O)CH₂CHF₂ |
| 214. | C(═O)OCH₃ | C(═O)CH₂CF₃ |
| 215. | C(═O)OCH₃ | C(═O)C₆H₅ |
| 216. | C(═O)OCH₃ | C(═O)CH₂C₆H₅ |
| 217. | C(═O)OCH₃ | C(═O)NH₂ |
| 218. | C(═O)OCH₃ | C(═O)NHCH₃ |
| 219. | C(═O)OCH₃ | C(═O)NHCH₂CH₃ |
| 220. | C(═O)OCH₃ | C(═O)NHCH₂CH₂CH₃ |
| 221. | C(═O)OCH₃ | C(═O)NHCHF₂ |
| 222. | C(═O)OCH₃ | C(═O)NHCF₃ |
| 223. | C(═O)OCH₃ | C(═O)NHCH₂CHF₂ |
| 224. | C(═O)OCH₃ | C(═O)NHCH₂CF₃ |
| 225. | C(═O)OCH₂CH₃ | H |
| 226. | C(═O)OCH₂CH₃ | CH₃ |
| 227. | C(═O)OCH₂CH₃ | CH₂CH₃ |
| 228. | C(═O)OCH₂CH₃ | CH₂CH₂CH₃ |
| 229. | C(═O)OCH₂CH₃ | CH(CH₃)₂ |
| 230. | C(═O)OCH₂CH₃ | CH₂CH₂CH₂CH₃ |
| 231. | C(═O)OCH₂CH₃ | CH₂CH(CH₃)₂ |
| 232. | C(═O)OCH₂CH₃ | CHF₂ |
| 233. | C(═O)OCH₂CH₃ | CF₃ |
| 234. | C(═O)OCH₂CH₃ | CH₂CF₃ |
| 235. | C(═O)OCH₂CH₃ | CH₂C₆H₅ |
| 236. | C(═O)OCH₂CH₃ | C₆H₅ |
| 237. | C(═O)OCH₂CH₃ | 4-Cl—C₆H₄ |
| 238. | C(═O)OCH₂CH₃ | 2,4-Cl₂—C₆H₃ |
| 239. | C(═O)OCH₂CH₃ | C(═O)H |
| 240. | C(═O)OCH₂CH₃ | C(═O)CH₃ |
| 241. | C(═O)OCH₂CH₃ | C(═O)CH₂CH₃ |
| 242. | C(═O)OCH₂CH₃ | C(═O)CH₂CH₂CH₃ |
| 243. | C(═O)OCH₂CH₃ | C(═O)CHF₂ |
| 244. | C(═O)OCH₂CH₃ | C(═O)CF₃ |
| 245. | C(═O)OCH₂CH₃ | C(═O)CH₂CHF₂ |
| 246. | C(═O)OCH₂CH₃ | C(═O)CH₂CF₃ |
| 247. | C(═O)OCH₂CH₃ | C(═O)C₆H₅ |
| 248. | C(═O)OCH₂CH₃ | C(═O)CH₂C₆H₅ |
| 249. | C(═O)OCH₂CH₃ | C(═O)NH₂ |
| 250. | C(═O)OCH₂CH₃ | C(═O)NHCH₃ |
| 251. | C(═O)OCH₂CH₃ | C(═O)NHCH₂CH₃ |
| 252. | C(═O)OCH₂CH₃ | C(═O)NHCH₂CH₂CH₃ |
| 253. | C(═O)OCH₂CH₃ | C(═O)NHCHF₂ |
| 254. | C(═O)OCH₂CH₃ | C(═O)NHCF₃ |
| 255. | C(═O)OCH₂CH₃ | C(═O)NHCH₂CHF₂ |
| 256. | C(═O)OCH₂CH₃ | C(═O)NHCH₂CF₃ |
| 257. | C(═O)NHCH₃ | H |
| 258. | C(═O)NHCH₃ | CH₃ |
| 259. | C(═O)NHCH₃ | CH₂CH₃ |
| 260. | C(═O)NHCH₃ | CH₂CH₂CH₃ |
| 261. | C(═O)NHCH₃ | CH(CH₃)₂ |
| 262. | C(═O)NHCH₃ | CH₂CH₂CH₂CH₃ |
| 263. | C(═O)NHCH₃ | CH₂CH(CH₃)₂ |
| 264. | C(═O)NHCH₃ | CHF₂ |
| 265. | C(═O)NHCH₃ | CF₃ |
| 266. | C(═O)NHCH₃ | CH₂CF₃ |
| 267. | C(═O)NHCH₃ | CH₂C₆H₅ |
| 268. | C(═O)NHCH₃ | C₆H₅ |
| 269. | C(═O)NHCH₃ | 4-Cl—C₆H₄ |
| 270. | C(═O)NHCH₃ | 2,4-Cl₂—C₆H₃ |
| 271. | C(═O)NHCH₃ | C(═O)H |
| 272. | C(═O)NHCH₃ | C(═O)CH₃ |
| 273. | C(═O)NHCH₃ | C(═O)CH₂CH₃ |
| 274. | C(═O)NHCH₃ | C(═O)CH₂CH₂CH₃ |
| 275. | C(═O)NHCH₃ | C(═O)CHF₂ |
| 276. | C(═O)NHCH₃ | C(═O)CF₃ |
| 277. | C(═O)NHCH₃ | C(═O)CH₂CHF₂ |
| 278. | C(═O)NHCH₃ | C(═O)CH₂CF₃ |
| 279. | C(═O)NHCH₃ | C(═O)C₆H₅ |
| 280. | C(═O)NHCH₃ | C(═O)CH₂C₆H₅ |
| 281. | C(═O)NHCH₃ | C(═O)NH₂ |
| 282. | C(═O)NHCH₃ | C(═O)NHCH₃ |
| 283. | C(═O)NHCH₃ | C(═O)NHCH₂CH₃ |
| 284. | C(═O)NHCH₃ | C(═O)NHCH₂CH₂CH₃ |
| 285. | C(═O)NHCH₃ | C(═O)NHCHF₂ |
| 286. | C(═O)NHCH₃ | C(═O)NHCF₃ |
| 287. | C(═O)NHCH₃ | C(═O)NHCH₂CHF₂ |
| 288. | C(═O)NHCH₃ | C(═O)NHCH₂CF₃ |
| 289. | C(═O)NHCH₂CH₃ | H |
| 290. | C(═O)NHCH₂CH₃ | CH₃ |
| 291. | C(═O)NHCH₂CH₃ | CH₂CH₃ |
| 292. | C(═O)NHCH₂CH₃ | CH₂CH₂CH₃ |
| 293. | C(═O)NHCH₂CH₃ | CH(CH₃)₂ |
| 294. | C(═O)NHCH₂CH₃ | CH₂CH₂CH₂CH₃ |
| 295. | C(═O)NHCH₂CH₃ | CH₂CH(CH₃)₂ |
| 296. | C(═O)NHCH₂CH₃ | CHF₂ |
| 297. | C(═O)NHCH₂CH₃ | CF₃ |
| 298. | C(═O)NHCH₂CH₃ | CH₂CF₃ |
| 299. | C(═O)NHCH₂CH₃ | CH₂C₆H₅ |
| 300. | C(═O)NHCH₂CH₃ | C₆H₅ |
| 301. | C(═O)NHCH₂CH₃ | 4-Cl—C₆H₄ |
| 302. | C(═O)NHCH₂CH₃ | 2,4-Cl₂—C₆H₃ |
| 303. | C(═O)NHCH₂CH₃ | C(═O)H |
| 304. | C(═O)NHCH₂CH₃ | C(═O)CH₃ |
| 305. | C(═O)NHCH₂CH₃ | C(═O)CH₂CH₃ |
| 306. | C(═O)NHCH₂CH₃ | C(═O)CH₂CH₂CH₃ |
| 307. | C(═O)NHCH₂CH₃ | C(═O)CHF₂ |
| 308. | C(═O)NHCH₂CH₃ | C(═O)CF₃ |
| 309. | C(═O)NHCH₂CH₃ | C(═O)CH₂CHF₂ |
| 310. | C(═O)NHCH₂CH₃ | C(═O)CH₂CF₃ |
| 311. | C(═O)NHCH₂CH₃ | C(═O)C₆H₅ |
| 312. | C(═O)NHCH₂CH₃ | C(═O)CH₂C₆H₅ |
| 313. | C(═O)NHCH₂CH₃ | C(═O)NH₂ |
| 314. | C(═O)NHCH₂CH₃ | C(═O)NHCH₃ |
| 315. | C(═O)NHCH₂CH₃ | C(═O)NHCH₂CH₃ |
| 316. | C(═O)NHCH₂CH₃ | C(═O)NHCH₂CH₂CH₃ |
| 317. | C(═O)NHCH₂CH₃ | C(═O)NHCHF₂ |
| 318. | C(═O)NHCH₂CH₃ | C(═O)NHCF₃ |
| 319. | C(═O)NHCH₂CH₃ | C(═O)NHCH₂CHF₂ |
| 320. | C(═O)NHCH₂CH₃ | C(═O)NHCH₂CF₃ |
| 321. | C(═O)N(CH₃)₂ | H |
| 322. | C(═O)N(CH₃)₂ | CH₃ |
| 323. | C(═O)N(CH₃)₂ | CH₂CH₃ |
| 324. | C(═O)N(CH₃)₂ | CH₂CH₂CH₃ |
| 325. | C(═O)N(CH₃)₂ | CH(CH₃)₂ |
| 326. | C(═O)N(CH₃)₂ | CH₂CH₂CH₂CH₃ |
| 327. | C(═O)N(CH₃)₂ | CH₂CH(CH₃)₂ |
| 328. | C(═O)N(CH₃)₂ | CHF₂ |
| 329. | C(═O)N(CH₃)₂ | CF₃ |
| 330. | C(═O)N(CH₃)₂ | CH₂CF₃ |
| 331. | C(═O)N(CH₃)₂ | CH₂C₆H₅ |
| 332. | C(═O)N(CH₃)₂ | C₆H₅ |
| 333. | C(═O)N(CH₃)₂ | 4-Cl—C₆H₄ |
| 334. | C(═O)N(CH₃)₂ | 2,4-Cl₂—C₆H₃ |
| 335. | C(═O)N(CH₃)₂ | C(═O)H |
| 336. | C(═O)N(CH₃)₂ | C(═O)CH₃ |
| 337. | C(═O)N(CH₃)₂ | C(═O)CH₂CH₃ |
| 338. | C(═O)N(CH₃)₂ | C(═O)CH₂CH₂CH₃ |
| 339. | C(═O)N(CH₃)₂ | C(═O)CHF₂ |
| 340. | C(═O)N(CH₃)₂ | C(═O)CF₃ |
| 341. | C(═O)N(CH₃)₂ | C(═O)CH₂CHF₂ |
| 342. | C(═O)N(CH₃)₂ | C(═O)CH₂CF₃ |
| 343. | C(═O)N(CH₃)₂ | C(═O)C₆H₅ |
| 344. | C(═O)N(CH₃)₂ | C(═O)CH₂C₆H₅ |
| 345. | C(═O)N(CH₃)₂ | C(═O)NH₂ |
| 346. | C(═O)N(CH₃)₂ | C(═O)NHCH₃ |
| 347. | C(═O)N(CH₃)₂ | C(═O)NHCH₂CH₃ |
| 348. | C(═O)N(CH₃)₂ | C(═O)NHCH₂CH₂CH₃ |
| 349. | C(═O)N(CH₃)₂ | C(═O)NHCHF₂ |
| 350. | C(═O)N(CH₃)₂ | C(═O)NHCF₃ |
| 351. | C(═O)N(CH₃)₂ | C(═O)NHCH₂CHF₂ |
| 352. | C(═O)N(CH₃)₂ | C(═O)NHCH₂CF₃ |
| 353. | C₆H₅ (phenyl) | H |
| 354. | C₆H₅ | CH₃ |
| 355. | C₆H₅ | CH₂CH₃ |
| 356. | C₆H₅ | CH₂CH₂CH₃ |
| 357. | C₆H₅ | CH(CH₃)₂ |
| 358. | C₆H₅ | CH₂CH₂CH₂CH₃ |
| 359. | C₆H₅ | CH₂CH(CH₃)₂ |
| 360. | C₆H₅ | CHF₂ |
| 361. | C₆H₅ | CF₃ |
| 362. | C₆H₅ | CH₂CF₃ |
| 363. | C₆H₅ | CH₂C₆H₅ |
| 364. | C₆H₅ | C₆H₅ |
| 365. | C₆H₅ | 4-Cl—C₆H₄ |
| 366. | C₆H₅ | 2,4-Cl₂—C₆H₃ |
| 367. | C₆H₅ | C(═O)H |
| 368. | C₆H₅ | C(═O)CH₃ |
| 369. | C₆H₅ | C(═O)CH₂CH₃ |
| 370. | C₆H₅ | C(═O)CH₂CH₂CH₃ |
| 371. | C₆H₅ | C(═O)CHF₂ |
| 372. | C₆H₅ | C(═O)CF₃ |
| 373. | C₆H₅ | C(═O)CH₂CHF₂ |
| 374. | C₆H₅ | C(═O)CH₂CF₃ |
| 375. | C₆H₅ | C(═O)C₆H₅ |
| 376. | C₆H₅ | C(═O)CH₂C₆H₅ |
| 377. | C₆H₅ | C(═O)NH₂ |
| 378. | C₆H₅ | C(═O)NHCH₃ |
| 379. | C₆H₅ | C(═O)NHCH₂CH₃ |
| 380. | C₆H₅ | C(═O)NHCH₂CH₂CH₃ |
| 381. | C₆H₅ | C(═O)NHCHF₂ |
| 382. | C₆H₅ | C(═O)NHCF₃ |
| 383. | C₆H₅ | C(═O)NHCH₂CHF₂ |
| 384. | C₆H₅ | C(═O)NHCH₂CF₃ |

Among the above compounds preference is given to compounds of formula I-1, I-2, I-3 and I-4, more preference to compounds I-1, I-2 and I-3, specifically to compounds I-1 and I-2 and very specifically to compounds I-1.

Compounds of formula I can be synthesized in analogy to methods described in WO 2013/063282. Scheme 1 shows the last step, which is a coupling reaction of a compound of formula II (with J being a leaving group such as halogen or —OSO₂CH₃, —OSO₂CF₃, —OS₂-p-C₆H₄—CH₃ and the like) and a Q-H ring (H being bound to the nitrogen ring atom that forms the attachment point of the respective formula Q-1, Q-2, Q-3, Q-4 or Q-5 to CR^{1a}R^{1b}). Suitable reaction conditions for such a conversion are described for example in WO 2013/063282 or by Lee et al. Journal of Medicinal Chemistry 2008, 51(22), 7216-7233.

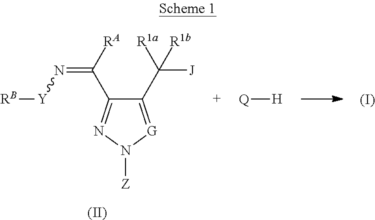

Compounds of formula Q-H can be prepared by methods described in WO 2013/063282.

Alternatively, compounds of formula I can be prepared by reductive amination of a compound of formula III with the Q-H ring as shown in scheme 2. Methods describing such a conversion are described for example by Li et al., Bioorganic & Medicinal Chemistry Letters 2010, 20(16), 4932-4935.

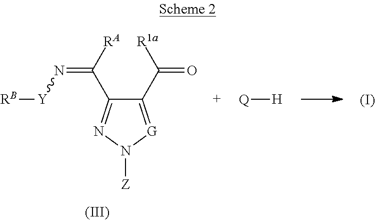

The synthesis of the Q-1-H radical of formula VII wherein X is CR^{3e}, L is a bond and A is OR^{a} as shown in scheme 3 is starting with a hydroxypiperidine of formula IV. Protection of the compound of formula IV with a suitable protecting group (PG), as for example described in WO 2004/009550 or U.S. Pat. No. 6,362,188, leads to a compound of formula V which subsequently is either alkylated or acylated at the hydroxy moiety, as described for example in US 2003/236250, WO 2006/104406 or US 2011/92475, to form a compound of formula VI. Deprotection as described for example in WO 2006/64196, WO 2004/104001 or US 2011/92475 leads to compounds of formula VII. Starting compound of formula IV can be synthesized from the respective hydroxypyridines, as described for example by Hall et al. Journal of the American Chemical Society 1958, 80, 6412-6419. Alternatively, compounds of formula IV can be synthesized from 1,3-dicyano-propane-2-ol under hydrogenation conditions, as described for example by Bowden et al. Journal of the Chemical Society 1952, 1164-1166.

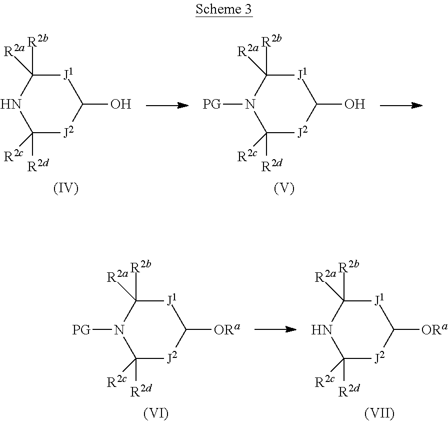

Q-1 radicals of the formula XIV wherein X is CR^{3e} and L-A is —CR^{d}═N—R^{c1} can be introduced, for example, as shown in scheme 4. Protection (PG is a protecting group) of compounds of formula VIII as described for example by Ting et al. Bioorganic and Medicinal Chemistry Letters 2001, 11(4), 491-494 gives compounds of formula IX. Oxidation of the latter as described for example by Ting et al. Bioorganic and Medicinal Chemistry Letters 2001, 11(4), 491-494 or in US 2008/300242 leads to compounds of formula X. Introduction of an R^{d} radical can be accomplished by the addition of an organometallic reagent as for example described in WO 2012/092827, WO 2005/026145 or WO 2012/018668 to yield compounds of formula XI. Oxidation of the latter in analogy to the methods as described for example by Ting et al. Bioorganic and Medicinal Chemistry Letters 2001, 11(4), 491-494 leads to compounds of formula XII. Amination of compounds of formula XII as described for example in WO 2008/118718 gives compounds of formula XIII. Similarly, compounds of formula X may be subjected to the same conditions to give compounds of formula XIII with R^{d}═H. Deprotection of compounds of formula XIII as for example described by Ivobe et al. Chemical & Pharmaceutical Bulletin, 2001, 49(7), 822-829 leads to compounds of formula XIV. Starting compound of formula IV can be synthesized from the respective hydroxymethylpyridines by hydrogenation as for example described in US 2003/55244. Q-1 radicals wherein X is CR^{3e} and L-A is —C(═N—N(R^{c1})R^{c2})R^{d} or —C(═NR^{c1})N(R^{c2})R^{c3} can be introduced analogously.

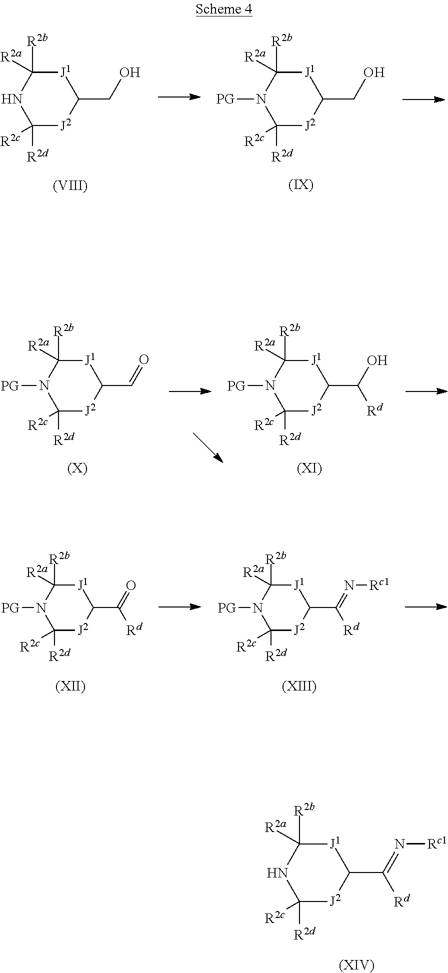

Q-1-radicals of the formula XVIII wherein X is N and L-A is a —CR^{d}═N—R^{c1} can be introduced as shown in scheme 5. Piperazine compounds of formula XV are protected as described for example by Kunieda et al. Tetrahedron Letters 1982, 23(11), 1159-1160. Introduction of an isocyanate as described for example in WO 2007/056170 leads to compounds of formula XVII (with R^{d}═H) which leads to compounds of formula XVIII upon deprotection, as described for example in WO 2007/056170. Compounds of formula XVII where R^{d} is not hydrogen can be prepared as described for example in WO 2003/103661, Areschka et al. European Journal of Medicinal Chemistry 1976, 11(2), 125-131, Macit et al. Synthesis and Reactivity in Inorganic and Metal-Organic Chemistry 1998, 28(5), 833-841, US 2007/0173495 or Moreau et al. European Journal of Medicinal Chemistry 1977, 12(4), 365-9. Q-1 radicals wherein X is CR^{3e} and L-A is —C(═N—N(R^{c1})R^{c2})R^{d} or —C(═NR^{c1})N(R^{c2})R^{c3} can be introduced analogously.

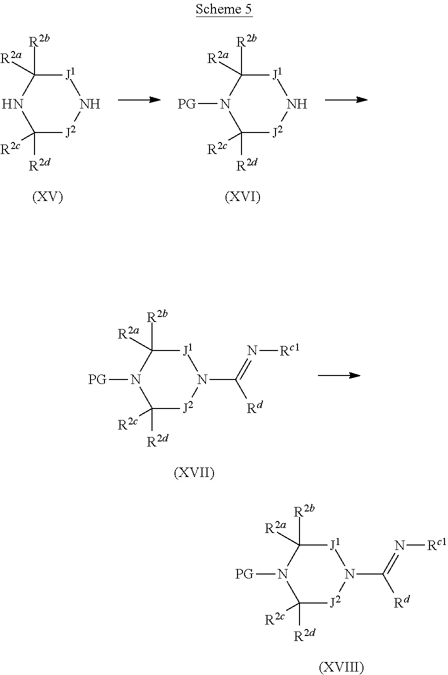

Q-2 radicals of the formula XXII can be introduced as shown in scheme 6. Piperidinones of formula XIX are protected at the N-terminus as described for example by Bridges et al. Bioorganic & Medicinal Chemistry Letters 2008, 18(20), 5439-5442 to give compounds of formula XX. Introduction of the B-moiety, followed by deprotection yields compounds of formula XXI and XXII, respectively. Transformation into B═S can be done by reaction with Lawesson's reagent as described for example in WO 2012/051117. Conversion into B═CR^{d1}R^{d2} can be carried out as described for example in US 2005/0261322. Conversion into B═NR¹ can be carried out as described for example by Xu et al. Bioorganic & Medicinal Chemistry Letters 2010, 20(9), 2942-2945. Deprotection can be achieved by using standard procedures as described for example in WO 2007/056170.

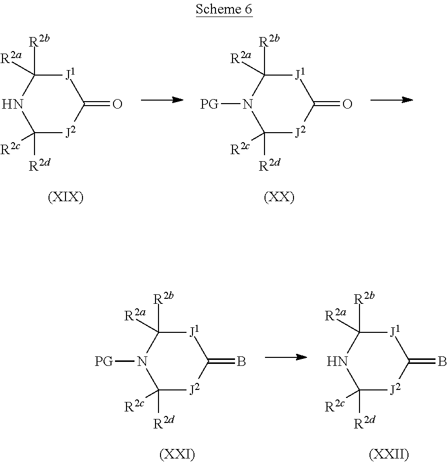

Q-3 radicals of the formula XXVII can be introduced as shown in scheme 7 from thiomorpholinones of formula XXIII by protection of compounds XXIII as described for example in WO 2008/083038 or in WO 99/09027 to give compounds of formula XXIV. Conversion into compounds of formula XXV, XXVI and XXVII can be carried out by methods as described in WO 2011/029537.

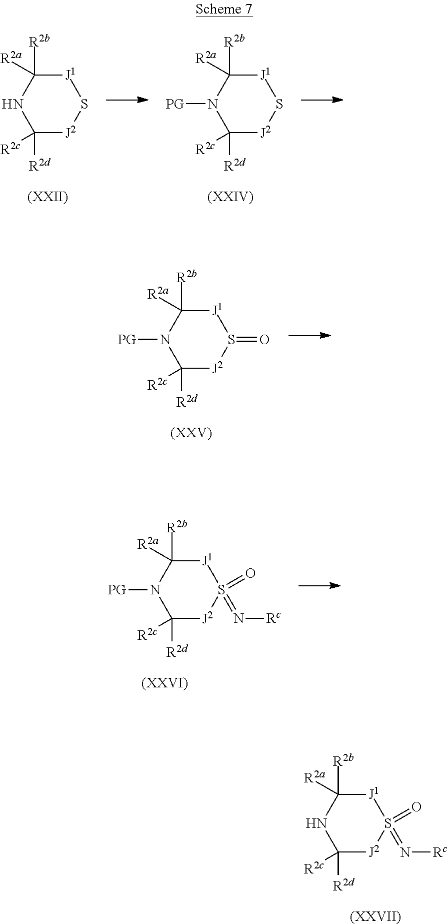

Compounds of formula I carrying a radical Q-4 (below termed XXIX) can be prepared as shown in scheme 8 from compounds of formula II as described for example by D'Auria et al. European Journal of Organic Chemistry 2009, (6), 932-937 by reacting the derived azide of formula XXVIII with an olefin.

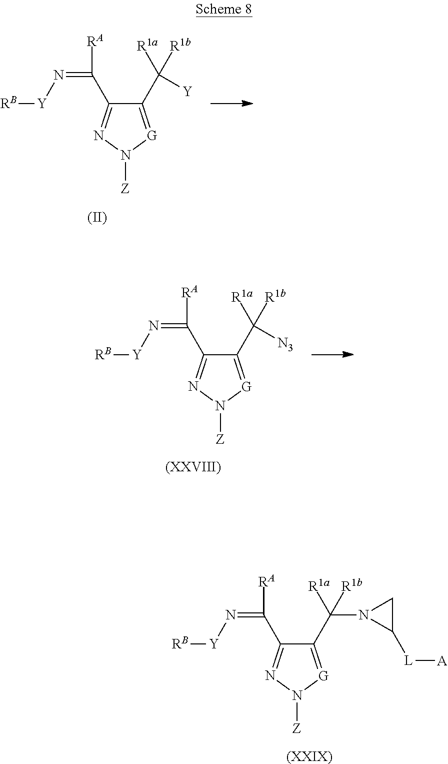

Compounds of formula I carrying a radical Q-5 can be prepared in analogy to methods described in schemes 1 and 2 above. Suitable methods for preparing Q-H radicals of type Q-5 have been described for example by Ghorai, Manas K. et al.; Journal of Organic Chemistry (2009), 74(18), 7013-7022, or in DE 105498, CN 1031699, JP 06056806 or JP 2000128872.

Compounds of formula I can also be prepared from compounds of formula XXVII by oxime formation, as for example described in WO 2008/118718 by amination. Compounds of formula XXVII can be obtained by oxidation of compounds of formula XXXVI, as for example described by Ting et al. Bioorganic and Medicinal Chemistry Letters 2001, 11(4), 491-494. Compounds of formula XXXVI can be obtained by reduction of compounds of formula XXXV, as for example described by Poon, Steve F. et al, Journal of Medicinal Chemistry, 52(21), 6535-6538; 2009. Selective reduction of compounds of formula XXXV may lead to compounds of formula XXXVII directly, as for example described in US 20100094006. Compounds of formula XXXV can be obtained by reductive amination of compounds of formula XXXIV, as for example described in WO 2010047956 or in WO 2012158413. Compounds of formula XXXIV can be obtained by a cyclization reaction of a compound of formula XXXIII with a subsequent formylation reaction in one pot, as for example described in WO 2010047956 or by Bratenko, M. K. et al, Chemistry of Heterocyclic Compounds (New York, N.Y., United States), 45(12), 1464-1468; 2009. Hydrazone compounds of formula XXXIII can be obtained by reaction of a ketoester of formula XXX with a hydrazine of formula XXXI, as for example described in WO 2010047956.

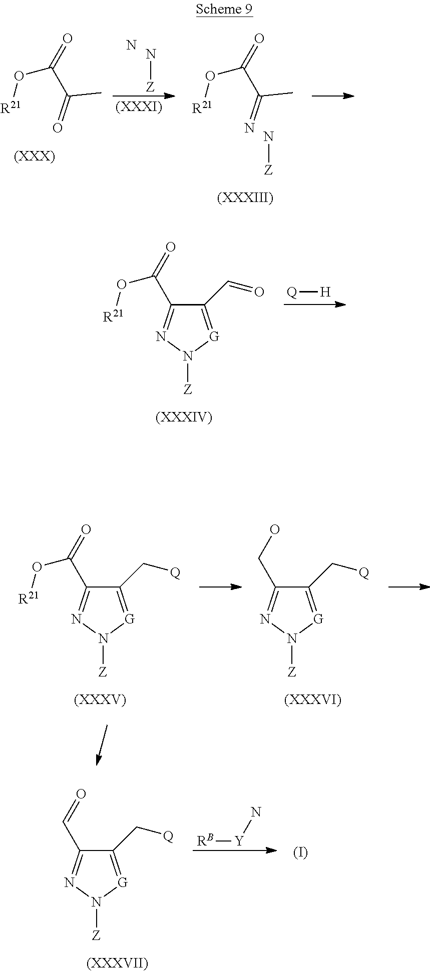

Compounds of formula XXXVII with G=N can be obtained as for example shown in scheme 10, by a reaction sequence described by Sekily et al, Jouenal of Chemical Research, 771-773, 2006. Starting from dehydro ascorbic acid derivative of formula XXXVIII, reaction with hydrazine Z—NHNH₂ leads to compound XXXIX which then is oximated to compound XXXX. Ring closure with acetic anhydride or Bromine in water led to triazine compound of formula XXXXI. Ring opening with the amine Q-H leads to amide compounds of formula XXXXII which then is reduced to the amine, as for example described in WO 2008/017932. Glycol cleavage of compounds of formula XXXXIII leads to compounds of formula XXXVII.

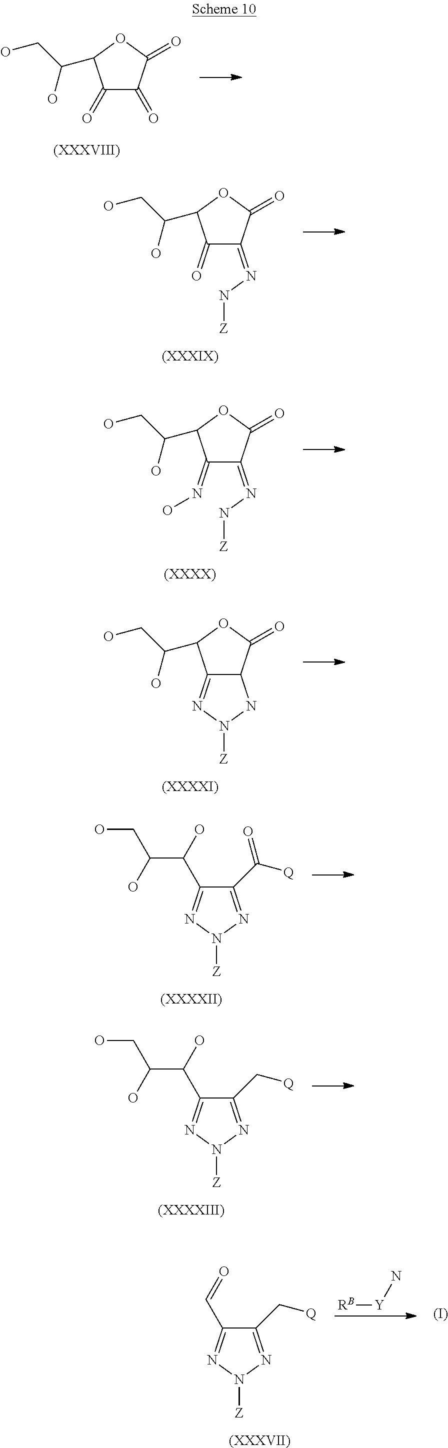

As a rule, the compounds of formula (I) including their stereoisomers, salts, and N-oxides, and their precursors in the synthesis process, can be prepared by the methods described above. If individual compounds can not be prepared via the above-described routes, they can be prepared by derivatization of other compounds (I) or the respective precursor or by customary modifications of the synthesis routes described. For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

Due to their excellent activity, the compounds of the present invention may be used for controlling invertebrate pests.

Accordingly, the present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of the present invention or a composition as defined above. The invention also relates to the use of a compound of the invention, of a stereoisomer and/or of an agriculturally or veterinarily acceptable salt thereof for combating invertebrate pests

Preferably, the method of the invention serves for protecting plant propagation material (such as seed) and the plant which grows therefrom from invertebrate pest attack or infestation and comprises treating the plant propagation material (such as seed) with a pesticidally effective amount of a compound of the present invention as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

Alternatively preferably, the method of the invention serves for protecting plants from attack or infestation by invertebrate pests, which method comprises treating the plants with a pesticidally effective amount of at least one compound of the invention, a stereoisomer thereof and/or at least one agriculturally acceptable salt thereof.

In the sense of the present invention, “invertebrate pests” are preferably selected from arthropods and nematodes, more preferably from harmful insects, arachnids and nematodes, and even more preferably from insects, acarids and nematodes. In the sense of the present invention, “invertebrate pests” are most preferably insects.

The invention further provides an agricultural composition for combating invertebrate pests, which comprises such an amount of at least one compound according to the invention and at least one inert liquid and/or solid agronomically acceptable carrier that has a pesticidal action and, if desired, at least one surfactant.

Such a composition may comprise a single active compound of the present invention or a mixture of several active compounds of the present invention. The composition according to the present invention may comprise an individual isomer or mixtures of isomers or a salt as well as individual tautomers or mixtures of tautomers.

The compounds of the present invention, including their salts, stereoisomers and tautomers, are in particular suitable for efficiently controlling arthropodal pests such as arachnids, myriapedes and insects as well as nematodes. They are especially suitable for efficiently combating or controlling the following pests:

insects from the order of the lepidopterans (Lepidoptera), for example Acronicta major, Adoxophyes orana, Aedia leucomelas, Agrotis spp. such as Agrotis fucosa, Agrotis segetum, Agrotis ipsilon; Alabama argillacea, Anticarsia gemmatalis, Anticarsia spp., Argyresthia conjugella, Autographa gamma, Barathra brassicae, Bucculatrix thurberiella, Bupalus piniarius, Cacoecia murinana, Cacoecia podana, Capua reticulana, Carpocapsa pomonella, Cheimatobia brumata, Chilo spp. such as Chilo suppressalis; Choristoneura fumiferana, Choristoneura occidentalis, Cirphis unipuncta, Clysia ambiguella, Cnaphalocerus spp., Cydia pomonella, Dendrolimus pini, Diaphania nitidalis, Diatraea grandiosella, Earias insulana, Elasmopalpus lignosellus, Ephestia cautella, Ephestia kuehniella, Eupoecilia ambiguella, Euproctis chrysorrhoea, Euxoa spp., Evetria bouliana, Feltia spp. such as Feltia subterranean; Galleria mellonella, Grapholitha funebrana, Grapholitha molesta, Helicoverpa spp. such as Helicoverpa armigera, Helicoverpa zea; Heliothis spp. such as Heliothis armigera, Heliothis virescens, Heliothis zea; Hellula undalis, Hibernia defoliaria, Hofmannophila pseudospretella, Homona magnanima, Hyphantria cunea, Hyponomeuta padella, Hyponomeuta malinellus, Keiferia lycopersicella, Lambdina fiscellaria, Laphygma spp. such as Laphygma exigua; Leucoptera coffeella, Leucoptera scitella, Lithocolletis blancardella, Lithophane antennata, Lobesia botrana, Loxagrotis albicosta, Loxostege sticticalis, Lymantria spp. such as Lymantria dispar, Lymantria monacha; Lyonetia clerkella, Malacosoma neustria, Mamestra spp. such as Mamestra brassicae; Mocis repanda, Mythimna separata, Orgyia pseudotsugata, Oria spp., Ostrinia spp. such as Ostrinia nubilalis; Oulema oryzae, Panolis flammea, Pectinophora spp. such as Pectinophora gossypiella; Peridroma saucia, Phalera bucephala, Phthorimaea spp. such as Phthorimaea operculella; Phyllocnistis citrella, Pieris spp. such as Pieris brassicae, Pieris rapae; Plathypena scabra, Plutella maculipennis, Plutella xylostella, Prodenia spp., Pseudaletia spp., Pseudoplusia includens, Pyrausta nubilalis, Rhyacionia frustrana, Scrobipalpula absoluta, Sitotroga cerealella, Sparganothis pilleriana, Spodoptera spp. such as Spodoptera frugiperda, Spodoptera littoralis, Spodoptera litura; Thaumatopoea pityocampa, Thermesia gemmatalis, Tinea pellionella, Tineola bisselliella, Tortrix viridana, Trichoplusia spp. such as Trichoplusia ni; Tuta absoluta, and Zeiraphera canadensis,
beetles (Coleoptera), for example Acanthoscehdes obtectus, Adoretus spp., Agelastica alni, Agrilus sinuatus, Agriotes spp. such as Agriotes fuscicollis, Agriotes lineatus, Agriotes obscurus; Amphimallus solstitialis, Anisandrus dispar, Anobium punctatum, Anomala rufocuprea, Anoplophora spp. such as Anoplophora glabripennis; Anthonomus spp. such as Anthonomus grandis, Anthonomus pomorum; Anthrenus spp., Aphthona euphoridae, Apogonia spp., Athous haemorrhoidalis, Atomaria spp. such as Atomaria linearis; Attagenus spp., Aulacophora femoralis, Blastophagus piniperda, Blitophaga undata, Bruchidius obtectus, Bruchus spp. such as Bruchus lentis, Bruchus pisorum, Bruchus rufimanus; Byctiscus betulae, Callosobruchus chinensis, Cassida nebulosa, Cerotoma trifurcata, Cetonia aurata, Ceuthorhynchus spp. such as Ceuthorrhynchus assimilis, Ceuthorrhynchus napi; Chaetocnema tibialis, Cleonus mendicus, Conoderus spp. such as Conoderus vespertinus; Cosmopolites spp., Costelytra zealandica, Crioceris asparagi, Cryptorhynchus lapathi, Ctenicera ssp. such as Ctenicera destructor; Curculio spp., Dectes texanus, Dermestes spp., Diabrotica spp. such as Diabrotica 12-punctata Diabrotica speciosa, Diabrotica longicornis, Diabrotica semipunctata, Diabrotica virgifera; Epilachna spp. such as Epilachna varivestis, Epilachna vigintioctomaculata; Epitrix spp. such as Epitrix hirtipennis; Eutinobothrus brasiliensis, Faustinus cubae, Gibbium psylloides, Heteronychus arator, Hylamorpha elegans, Hylobius abietis, Hylotrupes bajulus, Hypera brunneipennis, Hypera postica, Hypothenemus spp., Ips typographus, Lachnosterna consanguinea, Lema bilineata, Lema melanopus, Leptinotarsa spp. such as Leptinotarsa decemlineata; Limonius californicus, Lissorhoptrus oryzophilus, Lissorhoptrus oryzophilus, Lixus spp., Lyctus spp. such as Lyctus bruneus; Melanotus communis, Meligethes spp. such as Meligethes aeneus; Melolontha hippocastani, Melolontha melolontha, Migdolus spp., Monochamus spp. such as Monochamus alternatus; Naupactus xanthographus, Niptus hololeucus, Oryctes rhinoceros, Oryzaephilus surinamensis, Otiorrhynchus sulcatus, Otiorrhynchus ovatus, Otiorrhynchus sulcatus, Oulema oryzae, Oxycetonia jucunda, Phaedon cochleariae, Phyllobius pyri, Phyllopertha horticola, Phyllophaga spp., Phyllotreta spp. such as Phyllotreta chrysocephala, Phyllotreta nemorum, Phyllotreta striolata; Phyllophaga spp., Phyllopertha horticola, Popillia japonica, Premnotrypes spp., Psylliodes chrysocephala, Ptinus spp., Rhizobius ventralis, Rhizopertha dominica, Sitona lineatus, Sitophilus spp. such as Sitophilus granaria, Sitophilus zeamais; Sphenophorus spp. such as Sphenophorus levis; Sternechus spp. such as Sternechus subsignatus; Symphyletes spp., Tenebrio molitor, Tribolium spp. such as Tribolium castaneum; Trogoderma spp., Tychius spp., Xylotrechus spp., and Zabrus spp. such as Zabrus tenebrioides,
flies, mosquitoes (Diptera), e.g. Aedes spp. such as Aedes aegypti, Aedes albopictus, Aedes vexans; Anastrepha ludens, Anopheles spp. such as Anopheles albimanus, Anopheles crucians, Anopheles freeborni, Anopheles gambiae, Anopheles leucosphyrus, Anopheles maculipennis, Anopheles minimus, Anopheles quadrimaculatus, Anopheles sinensis; Bibio hortulanus, Calliphora erythrocephala, Calliphora vicina, Cerafitis capitata, Ceratitis capitata, Chrysomyia spp. such as Chrysomya bezziana, Chrysomya hominivorax, Chrysomya macellaria; Chrysops atlanticus, Chrysops discalis, Chrysops silacea, Cochliomyia spp. such as Cochliomyia hominivorax; Contarinia spp. such as Contarinia sorghicola; Cordylobia anthropophaga, Culex spp. such as Culex nigripalpus, Culex pipiens, Culex quinquefasciatus, Culex tarsalis, Culex tritaeniorhynchus; Culicoides furens, Culiseta inornata, Culiseta melanura, Cuterebra spp., Dacus cucurbitae, Dacus oleae, Dasineura brassicae, Delia spp. such as Delia antique, Delia coarctata, Delia platura, Delia radicum; Dermatobia hominis, Drosophila spp., Fannia spp. such as Fannia canicularis; Gastraphilus spp. such as Gasterophilus intestinalis; Geomyza Tripunctata, Glossina fuscipes, Glossina morsitans, Glossina palpalis, Glossina tachinoides, Haematobia irritans, Haplodiplosis equestris, Hippelates spp., Hylemyia spp. such as Hylemyia platura; Hypoderma spp. such as Hypoderma lineata; Hyppobosca spp., Leptoconops torrens, Liriomyza spp. such as Liriomyza sativae, Liriomyza trifolii; Lucilia spp. such as Lucilia caprina, Lucilia cuprina, Lucilia sericata; Lycoria pectoralis, Mansonia titillanus, Mayetiola spp. such as Mayetiola destructor; Musca spp. such as Musca autumnalis, Musca domestica; Muscina stabulans, Oestrus spp. such as Oestrus ovis; Opomyza florum, Oscinella spp. such as Oscinella frit; Pegomya hysocyami, Phlebotomus argentipes, Phorbia spp. such as Phorbia antiqua, Phorbia brassicae, Phorbia coarctata; Prosimulium mixtum, Psila rosae, Psorophora columbiae, Psorophora discolor, Rhagoletis cerasi, Rhagoletis pomonella, Sarcophaga spp. such as Sarcophaga haemorrhoidalis; Simulium vittatum, Stomoxys spp. such as Stomoxys calcitrans; Tabanus spp. such as Tabanus atratus, Tabanus bovinus, Tabanus lineola, Tabanus similis; Tannia spp., Tipula oleracea, Tipula paludosa, and Wohlfahrtia spp.,
thrips (Thysanoptera), e.g. Baliothrips biformis, Dichromothrips corbetti, Dichromothrips ssp., Enneothrips flavens, Frankliniella spp. such as Frankliniella fusca, Frankliniella occidentalis, Frankliniella tritici; Heliothrips spp., Hercinothrips femoralis, Kakothrips spp., Rhipiphorothrips cruentatus, Scirtothrips spp. such as Scirtothrips citri; Taeniothrips cardamoni, Thrips spp. such as Thrips oryzae, Thrips palmi, Thrips tabaci;
termites (Isoptera), e.g. Calotermes flavicollis, Coptotermes formosanus, Heterotermes aureus, Heterotermes longiceps, Heterotermes tenuis, Leucotermes flavipes, Odontotermes spp., Reticulitermes spp. such as Reticulitermes speratus, Reticulitermes flavipes, Reticulitermes grassei, Reticulitermes lucifugus, Reticulitermes santonensis, Reticulitermes virginicus; Termes natalensis,
cockroaches (Blattaria-Blattodea), e.g. Acheta domesticus, Blatta orientalis, Blattella asahinae, Blattella germanica, Gryllotalpa spp., Leucophaea maderae, Locusta spp., Melanoplus spp., Periplaneta americana, Periplaneta australasiae, Periplaneta brunnea, Periplaneta fuligginosa, Periplaneta japonica,
bugs, aphids, leafhoppers, whiteflies, scale insects, cicadas (Hemiptera), e.g. Acrosternum spp. such as Acrosternum hilare; Acyrthosipon spp. such as Acyrthosiphon onobrychis, Acyrthosiphon pisum; Adelges laricis, Aeneolamia spp., Agonoscena spp., Aleurodes spp., Aleurolobus barodensis, Aleurothrixus spp., Amrasca spp., Anasa tristis, Antestiopsis spp., Anuraphis cardui, Aonidiella spp., Aphanostigma piri, Aphidula nasturtii, Aphis spp. such as Aphis fabae, Aphis forbesi, Aphis gossypii, Aphis grossulariae, Aphis pomi, Aphis sambuci, Aphis schneideri, Aphis spiraecola; Arboridia apicalis, Arilus critatus, Aspidiella spp., Aspidiotus spp., Atanus spp., Aulacorthum solani, Bemisia spp. such as Bemisia argentifolii, Bemisia tabaci; Blissus spp. such as Blissus leucopterus; Brachycaudus cardui, Brachycaudus helichrysi, Brachycaudus persicae, Brachycaudus prunicola, Brachycolus spp., Brevicoryne brassicae, Calligypona marginata, Calocoris spp., Campylomma livida, Capitophorus horni, Carneocephala fulgida, Cavelerius spp., Ceraplastes spp., Ceratovacuna lanigera, Cercopidae, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as Cimex hemipterus, Cimex lectularius; Coccomytilus halli, Coccus spp., Creontiades dilutus, Cryptomyzus ribis, Cryptomyzus ribis, Cyrtopeltis notatus, Dalbulus spp., Dasynus piperis, Dialeurades spp., Diaphorina spp., Diaspis spp., Dichelops furcatus, Diconocoris hewetti, Doralis spp., Dreyfusia nordmannianae, Dreyfusia piceae, Drosicha spp., Dysaphis spp. such as Dysaphis plantaginea, Dysaphis pyri, Dysaphis radicola; Dysaulacorthum pseudosolani, Dysdercus spp. such as Dysdercus cingulatus, Dysdercus intermedius; Dysmicoccus spp., Empoasca spp. such as Empoasca fabae, Empoasca solana; Eriosoma spp., Erythroneura spp., Eurygaster spp. such as Eurygaster integriceps; Euscelis bilobatus, Euschistus spp. such as Euschistuos heros, Euschistus impictiventris, Euschistus servus; Geococcus coffeae, Halyomorpha spp. such as Halyomorpha halys; Heliopeltis spp., Homalodisca coagulata, Horcias nobilellus, Hyalopterus pruni, Hyperomyzus lactucae, Icerya spp., Idiocerus spp., Idioscopus spp., Laodelphax striatellus, Lecanium spp., Lepidosaphes spp., Leptocorisa spp., Leptoglossus phyllopus, Lipaphis erysimi, Lygus spp. such as Lygus hesperus, Lygus lineolaris, Lygus pratensis; Macropes excavatus, Macrosiphum spp. such as Macrosiphum rosae, Macrosiphum avenae, Macrosiphum euphorbiae; Mahanarva fimbriolata, Megacopta cribraria, Megoura viciae, Melanaphis pyrarius, Melanaphis sacchari, Metcafiella spp., Metopolophium dirhodum, Miridae spp., Monellia costalis, Monelliopsis pecanis, Myzus spp. such as Myzus ascalonicus, Myzus cerasi, Myzus persicae, Myzus varians; Nasonovia ribis-nigri, Nephotettix spp. such as Nephotettix malayanus, Nephotettix nigropictus, Nephotettix parvus, Nephotettix virescens; Nezara spp. such as Nezara viridula; Nilaparvata lugens, Oebalus spp., Oncometopia spp., Orthezia praelonga, Parabemisia myricae, Paratrioza spp., Parlatoria spp., Pemphigus spp. such as Pemphigus bursarius; Pentomidae, Peregrinus maidis, Perkinsiella saccharicida, Phenacoccus spp., Phloeomyzus passerinii, Phorodon humuli, Phylloxera spp., Piesma quadrata, Piezodorus spp. such as Piezodorus guildinii, Pinnaspis aspidistrae, Planococcus spp., Protopulvinaria pyriformis, Psallus seriatus, Pseudacysta persea, Pseudaulacaspis pentagona, Pseudococcus spp. such as Pseudococcus comstocki; Psylla spp. such as Psylla mali, Psylla piri; Pteromalus spp., Pyrilla spp., Quadraspidiotus spp., Quesada gigas, Rastrococcus spp., Reduvius senilis, Rhodnius spp., Rhopalomyzus ascalonicus, Rhopalosiphum spp. such as Rhopalosiphum pseudobrassicas, Rhopalosiphum insertum, Rhopalosiphum maidis, Rhopalosiphum padi; Sagatodes spp., Sahlbergella singularis, Saissetia spp., Sappaphis mala, Sappaphis mali, Scaphoides titanus, Schizaphis graminum, Schizoneura lanuginosa, Scotinophora spp., Selenaspidus articulatus, Sitobion avenae, Sogata spp., Sogatella furcifera, Solubea insularis, Stephanitis nashi, Stictocephala festina, Tenalaphara malayensis, Thyanta spp. such as Thyanta perditor; Tibraca spp., Tinocallis caryaefoliae, Tomaspis spp., Toxoptera spp. such as Toxoptera aurantii; Trialeurodes spp. such as Trialeurodes vaporariorum; Triatoma spp., Trioza spp., Typhlocyba spp., Unaspis spp. such as Unaspis yanonensis; and Viteus vitifolii,
ants, bees, wasps, sawflies (Hymenoptera), e.g. Athalia rosae, Atta capiguara, Atta cephalotes, Atta cephalotes, Atta laevigata, Atta robusta, Atta sexdens, Atta texana, Bombus spp., Camponotus floridanus, Crematogaster spp., Dasymutilla occidentalis, Diprion spp., Dolichovespula maculata, Hoplocampa spp. such as Hoplocampa minuta, Hoplocampa testudinea; Lasius spp. such as Lasius niger, Linepithema humile, Monomorium pharaonis, Paravespula germanica, Paravespula pennsylvanica, Paravespula vulgaris, Pheidole megacephala, Pogonomyrmex barbatus, Pogonomyrmex californicus, Polistes rubiginosa, Solenopsis geminata, Solenopsis invicta, Solenopsis richteri, Solenopsis xyloni, Vespa spp. such as Vespa crabro, and Vespula squamosa,
crickets, grasshoppers, locusts (Orthoptera), e.g. Acheta domestica, Calliptamus italicus, Chortoicetes terminifera, Dociostaurus maroccanus, Gryllotalpa africana, Gryllotalpa gryllotalpa, Hieroglyphus daganensis, Kraussaria angulifera, Locusta migratoria, Locustana pardalina, Melanoplus bivittatus, Melanoplus femurrubrum, Melanoplus mexicanus, Melanoplus sanguinipes, Melanoplus spretus, Nomadacris septemfasciata, Oedaleus senegalensis, Schistocerca americana, Schistocerca gregaria, Tachycines asynamorus, and Zonozerus variegatus,
arachnids (Arachnida), such as acari, e.g. of the families Argasidae, Ixodidae and Sarcoptidae, such as Amblyomma spp. (e.g. Amblyomma americanum, Amblyomma variegatum, Amblyomma maculatum), Argas spp. (e.g. Argas persicus), Boophilus spp. (e.g. Boophilus annulatus, Boophilus decoloratus, Boophilus microplus), Dermacentor silvarum, Dermacentor andersoni, Dermacentor variabilis, Hyalomma spp. (e.g. Hyalomma truncatum), Ixodes spp. (e.g. Ixodes ricinus, Ixodes rubicundus, Ixodes scapularis, Ixodes holocyclus, Ixodes pacificus), Ornithodorus spp. (e.g. Ornithodorus moubata, Ornithodorus hermsi, Ornithodorus turicata), Ornithonyssus bacoti, Otobius megnini, Dermanyssus gallinae, Psoroptes spp. (e.g. Psoroptes ovis), Rhipicephalus spp. (e.g. Rhipicephalus sanguineus, Rhipicephalus appendiculatus, Rhipicephalus evertsi), Rhizoglyphus spp., Sarcoptes spp. (e.g. Sarcoptes scabiei), and Eriophyidae spp. such as Acaria sheldoni, Aculops spp. (e.g. Aculops pelekassi) Aculus spp. (e.g. Aculus schlechtendali), Epitrimerus pyri, Phyllocoptruta oleivora and Eriophyes spp. (e.g. Eriophyes sheldoni); Tarsonemidae spp. such as Hemitarsonemus spp., Phytonemus pallidus and Polyphagotarsonemus latus, Stenotarsonemus spp.; Tenuipalpidae spp. such as Brevipalpus spp. (e.g. Brevipalpus phoenicis); Tetranychidae spp. such as Eotetranychus spp., Eutetranychus spp., Oligonychus spp., Tetranychus cinnabarinus, Tetranychus kanzawai, Tetranychus pacificus, Tetranychus telarius and Tetranychus urticae; Bryobia praetiosa, Panonychus spp. (e.g. Panonychus ulmi, Panonychus citri), Metatetranychus spp. and Oligonychus spp. (e.g. Oligonychus pratensis), Vasates lycopersici; Araneida, e.g. Latrodectus mactans, and Loxosceles reclusa. And Acarus siro, Chorioptes spp., Scorpio maurus
fleas (Siphonaptera), e.g. Ceratophyllus spp., Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica,
centipedes (Chilopoda), e.g. Geophilus spp., Scutigera spp. such as Scutigera coleoptrata;
millipedes (Diplopoda), e.g. Blaniulus guttulatus, Narceus spp.,
Earwigs (Dermaptera), e.g. forficula auricularia,
lice (Phthiraptera), e.g. Damalinia spp., Pediculus spp. such as Pediculus humanus capitis, Pediculus humanus corporis; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp.,
springtails (Collembola), e.g. Onychiurus ssp. such as Onychiurus armatus,

They are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species such as Aphelenchoides besseyi; Sting nematodes, Belonolaimus longicaudatus and other Belonolaimus species; Pine nematodes, Bursaphelenchus lignicolus Mamiya et Kiyohara, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus brachyurus, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species such as Tylenchulus semipenetrans; Dagger nematodes, Xiphinema species; and other plant parasitic nematode species.

Examples of further pest species which may be controlled by compounds of formula (I) include: from the class of the Bivalva, for example, Dreissena spp.; from the class of the Gastropoda, for example, Arion spp., Biomphalaria spp., Bulinus spp., Deroceras spp., Galba spp., Lymnaea spp., Oncomelania spp., Succinea spp.; from the class of the helminths, for example, Ancylostoma duodenale, Ancylostoma ceylanicum, Acylostoma braziliensis, Ancylostoma spp., Ascaris lumbricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti; from the order of the Isopoda, for example, Armadillidium vulgare, Oniscus asellus, Porcellio scaber; from the order of the Symphyla, for example, Scutigerella immaculata;

Further examples of pest species which may be controlled by compounds of formula (I) include: Anisoplia austriaca, Apamea spp., Austroasca viridigrisea, Baliothrips biformis, Caenorhabditis elegans, Cephus spp., Ceutorhynchus napi, Chaetocnema aridula, Chilo auricilius, Chilo indicus, Chilo polychrysus, Chortiocetes terminifera, Cnaphalocroci medinalis, Cnaphalocrosis spp., Colias eurytheme, Collops spp., Cornitermes cumulans, Creontiades spp., Cyclocephala spp., Dalbulus maidis, Deraceras reticulatum, Diatrea saccharalis, Dichelops furcatus, Dicladispa armigera, Diloboderus spp. such as Diloboderus abderus; Edessa spp., Epinotia spp., Formicidae, Geocoris spp., Globitermes sulfureus, Gryllotalpidae, Halotydeus destructor, Hipnodes bicolor, Hydrellia philippina, Julus spp., Laodelphax spp., Leptocorsia acuta, Leptocorsia oratorius, Liogenys fuscus, Lucillia spp., Lyogenys fuscus, Mahanarva spp., Maladera matrida, Marasmia spp., Mastotermes spp., Mealybugs, Megascelis ssp, Metamasius hemipterus, Microtheca spp., Mocis latipes, Murgantia spp., Mythemina separata, Neocapritermes opacus, Neocapritermes parvus, Neomegalotomus spp., Neotermes spp., Nymphula depunctalis, Oebalus pugnax, Orseolia spp. such as Orseolia oryzae; Oxycaraenus hyalinipennis, Plusia spp., Pomacea canaliculata, Procornitermes ssp, Procornitermes triacifer, Psylloides spp., Rachiplusia spp., Rhodopholus spp., Scaptocoris castanea, Scaptocoris spp., Scirpophaga spp. such as Scirpophaga incertulas, Scirpophaga innotata; Scotinophara spp. such as Scotinophara coarctata; Sesamia spp. such as Sesamia inferens, Sogaella frucifera, Solenapsis geminata, Spissistilus spp., Stalk borer, Stenchaetothrips biformis, Steneotarsonemus spinki, Sylepta derogata, Telehin licus, Trichostrongylus spp.

The compounds of the present invention, including their salts, stereoisomers and tautomers, are particularly useful for controlling insects, preferably sucking or piercing and chewing and biting insects such as insects from the genera Lepidoptera, Coleoptera and Hemiptera, in particular Lepidoptera, Coleoptera and true bugs.

The compounds of the present invention, including their salts, stereoisomers and tautomers, are moreover useful for controlling insects of the orders Thysanoptera, Diptera (especially flies, mosquitos), Hymenoptera (especially ants) and Isoptera (especially termites.

The compounds of the present invention, including their salts, stereoisomers and tautomers, are particularly useful for controlling insects of the orders Lepidoptera and Coleoptera.

The invention also relates to agrochemical compositions comprising an auxiliary and at least one compound I according to the invention.

An agrochemical composition comprises a pesticidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the species to be controlled, the treated cultivated plant or material, the climatic conditions and the specific compound I used.

The compounds I, their N-oxides and salts can be converted into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyland tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

i) Water-soluble concentrates (SL, LS)

10-60 wt % of a compound I according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible concentrates (DC)

5-25 wt % of a compound I according to the invention and I-10 wt % dispersant (e.g. polyvinylpyrrolidone) are dissolved in organic solvent (e.g. cyclohexanone) ad 100 wt %. Dilution with water gives a dispersion.

iii) Emulsifiable concentrates (EC)

15-70 wt % of a compound I according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

### iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

vi) Water-dispersible granules and water-soluble granules (WG, SG)

50-80 wt % of a compound I according to the invention are ground finely with addition of dispersants and wetting agents (e.g. sodium lignosulfonate and alcohol ethoxylate) ad 100 wt % and prepared as water-dispersible or water-soluble granules by means of technical appliances (e.g. extrusion, spray tower, fluidized bed). Dilution with water gives a stable dispersion or solution of the active substance.

vii) Water-dispersible powders and water-soluble powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

### iv) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water ad 100%. This mixture is stirred for 1 h to produce spontaneously a thermodynamically stable microemulsion.

### iv) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsules. The monomers amount to 1-10 wt %. The wt % relate to the total CS composition.

ix) Dustable powders (DP, DS)

1-10 wt % of a compound I according to the invention are ground finely and mixed intimately with solid carrier (e.g. finely divided kaolin) ad 100 wt %.

### x) Granules (GR, FG)

0.5-30 wt % of a compound I according to the invention is ground finely and associated with solid carrier (e.g. silicate) ad 100 wt %. Granulation is achieved by extrusion, spray-drying or the fluidized bed.

xi) Ultra-low volume liquids (UL)

1-50 wt % of a compound I according to the invention are dissolved in organic solvent (e.g. aromatic hydrocarbon) ad 100 wt %.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required. When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate. In a further embodiment, either individual components of the composition according to the invention or partially premixed components, e.g. components comprising compounds I and/or active substances from the groups M) or F) (see below), may be mixed by the user in a spray tank and further auxiliaries and additives may be added, if appropriate.

In a further embodiment, either individual components of the composition according to the invention or partially premixed components, e. g. components comprising compounds I and/or active substances from the groups M.1 to M.UN.X or F.I to F.XIII, can be applied jointly (e.g. after tank mix) or consecutively.

The following list M of pesticides, grouped according the Mode of Action Classification of the Insecticide Resistance Action Committee (IRAC), together with which the compounds according to the invention can be used and with which potential synergistic effects might be produced, is intended to illustrate the possible combinations, but not to impose any limitation:
M.1 Acetylcholine esterase (AChE) inhibitors from the class of
M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of
M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;
M.2. GABA-gated chloride channel antagonists such as:
M.2A cyclodiene organochlorine compounds, as for example endosulfan or chlordane; or
M.2B fiproles (phenylpyrazoles), as for example ethiprole, fipronil, flufiprole, pyrafluprole and pyriprole;
M.3 Sodium channel modulators from the class of
M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zetacypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin, metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or
M.3B sodium channel modulators such as DDT or methoxychlor;
M.4 Nicotinic acetylcholine receptor agonists (nAChR) from the class of
M.4A neonicotinoids, for example acteamiprid, chlothianidin, dinotefuran, imidacloprid, nitenpyram, thiacloprid and thiamethoxam; or the compounds
M.4A.1: 1-[(6-chloro-3-pyridinyl)methyl]-2,3,5,6,7,8-hexahydro-9-nitro-(5S,8R)-5,8-Epoxy-1H-imidazo[1,2-a]azepine; or
M.4A.2: 1-[(6-chloro-3-pyridyl)methyl]-2-nitro-1-[(E)-pentylideneamino]guanidine; or
M4.A.3: 1-[(6-chloro-3-pyridyl)methyl]-7-methyl-8-nitro-5-propoxy-3,5,6,7-tetrahydro-2H-imidazo[1,2-a]pyridine;
or M.4B nicotine.
M.5 Nicotinic acetylcholine receptor allosteric activators from the class of spinosyns, for example spinosad or spinetoram;
M.6 Chloride channel activators from the class of avermectins and milbemycins, for example abamectin, emamectin benzoate, ivermectin, lepimectin or milbemectin;
M.7 Juvenile hormone mimics, such as
M.7A juvenile hormone analogues as hydroprene, kinoprene and methoprene; or others as M.7B fenoxycarb or M.7C pyriproxyfen;
M.8 miscellaneous non-specific (multi-site) inhibitors, for example
M.8A alkyl halides as methyl bromide and other alkyl halides, or
M.8B chloropicrin, or M.8C sulfuryl fluoride, or M.8D borax, or M.8E tartar emetic;
M.9 Selective homopteran feeding blockers, for example
M.9B pymetrozine, or M.9C flonicamid;
M.10 Mite growth inhibitors, for example
M.10A clofentezine, hexythiazox and diflovidazin, or M.10B etoxazole;
M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;
M.12 Inhibitors of mitochondrial ATP synthase, for example
M.12A diafenthiuron, or
M.12B organotin miticides such as azocyclotin, cyhexatin or fenbutatin oxide, or M.12C propargite, or M.12D tetradifon;
M.13 Uncouplers of oxidative phosphorylation via disruption of the proton gradient, for example chlorfenapyr, DNOC or sulfluramid;
M.14 Nicotinic acetylcholine receptor (nAChR) channel blockers, for example nereistoxin analogues as bensultap, cartap hydrochloride, thiocyclam or thiosultap sodium;
M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;
M.16 Inhibitors of the chitin biosynthesis type 1, as for example buprofezin;
M.17 Moulting disruptors, Dipteran, as for example cyromazine;
M.18 Ecdyson receptor agonists such as diacylhydrazines, for example methoxyfenozide, tebufenozide, halofenozide, fufenozide or chromafenozide;
M.19 Octopamin receptor agonists, as for example amitraz;
M.20 Mitochondrial complex III electron transport inhibitors, for example
M.20A hydramethylnon, or M.20B acequinocyl, or M.20C fluacrypyrim;
M.21 Mitochondrial complex I electron transport inhibitors, for example
M.21A METI acaricides and insecticides such as fenazaquin, fenpyroximate, pyrimidifen, pyridaben, tebufenpyrad or tolfenpyrad, or M.21 B rotenone;
M.22 Voltage-dependent sodium channel blockers, for example
M.22A indoxacarb, or M.22B metaflumizone, or M.22C 1-[(E)-[2-(4-cyanophenyl)-1-[3-(trifluoromethyl)phenyl]ethylidene]amino]-3-[4-(difluoromethoxy)phenyl]urea;
M.23 Inhibitors of the of acetyl CoA carboxylase, such as Tetronic and Tetramic acid derivatives, for example spirodiclofen, spiromesifen or spirotetramat;
M.24 Mitochondrial complex IV electron transport inhibitors, for example
M.24A phosphine such as aluminium phosphide, calcium phosphide, phosphine or zinc phosphide, or M.24B cyanide.
M.25 Mitochondrial complex II electron transport inhibitors, such as beta-ketonitrile derivatives, for example cyenopyrafen or cyflumetofen;
M.28 Ryanodine receptor-modulators from the class of diamides, as for example flubendiamide, chlorantraniliprole (Rynaxypyr®), cyantraniliprole (Cyazypyr®), or the phthalamide compounds
M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and
M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound
M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound
M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.28.5a) to M.28.5l):
M.28.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide;
M.28.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide;
M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide;
M.28.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide;
M.28.5e) N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide;
M.28.5f) N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide;
M.28.5g) N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide;
M.28.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide;
M.28.5i) N-[2-(5-amino-1,3,4-thiadiazol-2-yl)-4-chloro-6-methyl-phenyl]-5-bromo-2-(3-chloro-2-pyridyl)pyrazole-3-carboxamide;
M.28.5j) 5-chloro-2-(3-chloro-2-pyridyl)-N-[2,4-dichloro-6-[(1-cyano-1-methyl-ethyl)carbamoyl]phenyl]pyrazole-3-carboxamide;
M.28.5k) 5-bromo-N-[2,4-dichloro-6-(methylcarbamoyl)phenyl]-2-(3,5-dichloro-2-pyridyl)pyrazole-3-carboxamide;
M.28.5l) N-[2-(tert-butylcarbamoyl)-4-chloro-6-methyl-phenyl]-2-(3-chloro-2-pyridyl)-5-(fluoromethoxy)pyrazole-3-carboxamide; or a compound selected from
M.28.6 N2-(1-cyano-1-methyl-ethyl)-N1-(2,4-dimethylphenyl)-3-iodo-phthalamide; or
M.28.7 3-chloro-N2-(1-cyano-1-methyl-ethyl)-N1-(2,4-dimethylphenyl)phthalamide;
M.UN.X insecticidal active compounds of unknown or uncertain mode of action, as for example afidopyropen, azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, pyflubumide or the compounds
M.UN.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
M.UN.X.2: 4-[5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide, or the compound
M.UN.X.3: 11-(4-chloro-2,6-dimethylphenyl)-12-hydroxy-1,4-dioxa-9-azadispiro[4.2.4.2]-tetradec-11-en-10-one, or the compound
M.UN.X.4: 3-(4′-fluoro-2,4-dimethylbiphenyl-3-yl)-4-hydroxy-8-oxa-1-azaspiro[4.5]dec-3-en-2-one, or the compound
M.UN.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, I-1582); or
M.UN.X.6; a compound selected from the group of
M.UN.X.6a) (E/Z)-N-[1-[(6-chloro-3-pyridyl)methyl]-2-pyridylidene]-2,2,2-trifluoro-acetamide;
M.UN.X.6b) (E/Z)-N-[1-[(6-chloro-5-fluoro-3-pyridyl)methyl]-2-pyridylidene]-2,2,2-trifluoro-acetamide;
M.UN.X.6c) (E/Z)-2,2,2-trifluoro-N-[1-[(6-fluoro-3-pyridyl)methyl]-2-pyridylidene]acetamide;
M.UN.X.6d) (E/Z)-N-[1-[(6-bromo-3-pyridyl)methyl]-2-pyridylidene]-2,2,2-trifluoro-acetamide;
M.UN.X.6e) (E/Z)-N-[1-[1-(6-chloro-3-pyridyl)ethyl]-2-pyridylidene]-2,2,2-trifluoro-acetamide;
M.UN.X.6f) (E/Z)-N-[1-[(6-chloro-3-pyridyl)methyl]-2-pyridylidene]-2,2-difluoroacetamide;
M.UN.X.6g) (E/Z)-2-chloro-N-[1-[(6-chloro-3-pyridyl)methyl]-2-pyridylidene]-2,2-difluoroacetamide;
M.UN.X.6h) (E/Z)-N-[1-[(2-chloropyrimidin-5-yl)methyl]-2-pyridylidene]-2,2,2-trifluoro-acetamide and
M.UN.X.6i) (E/Z)-N-[1-[(6-chloro-3-pyridyl)methyl]-2-pyridylidene]-2,2,3,3,3-pentafluoro-propanamide.); or of the compounds
M.UN.X.7: 3-[3-chloro-5-(trifluoromethyl)phenyl]-4-oxo-1-(pyrimidin-5-ylmethyl)pyrido[1,2-a]pyrimidin-1-ium-2-olate; or
M.UN.X.8: 8-chloro-N-[2-chloro-5-methoxyphenyl)sulfonyl]-6-trifluoromethyl)imidazo[1,2-a]pyridine-2-carboxamide; or
M.UN.X.9: 4-[5-(3,5-dichlorophenyl)-5-(trifluoromethyl)-4H-isoxazol-3-yl]-2-methyl-N-(1-oxothietan-3-yl)benzamide; or
M.UN.X.10: 5-[3-[2,6-dichloro-4-(3,3-dichloroallyloxy)phenoxy]propoxy]-1H-pyrazole.

The commercially available compounds of the group M listed above may be found in The Pesticide Manual, 15th Edition, C. D. S. Tomlin, British Crop Protection Council (2011) among other publications.

The quinoline derivative flometoquin is shown in WO2006/013896. The aminofuranone compounds flupyradifurone is known from WO 2007/115644. The sulfoximine compound sulfoxaflor is known from WO2007/149134. The pyrethroid momfluorothrin is known from U.S. Pat. No. 6,908,945. The pyrazole acaricide pyflubumide is known from WO2007/020986. The isoxazoline compounds have been described likewise M.UN.X.1 in WO2005/085216, M.UN.X2. in WO2009/002809 and in WO2011/149749 and the isoxazoline M.UN.X.9 in WO2013/050317. The pyripyropene derivative afidopyropen has been described in WO 2006/129714. The spiroketal-substituted cyclic ketoenol derivative M.UN.X.3 is known from WO2006/089633 and the biphenyl-substituted spirocyclic ketoenol derivative M.UN.X.4 from WO2008/067911. Finally triazoylphenylsulfide like M.UN.X.5 have been described in WO2006/043635 and biological control agents on basis of bacillus firmus in WO2009/124707. The neonicotionids 4A.1 is known from WO20120/069266 and WO2011/06946, the M.4.A.2 from WO2013/003977, the M4.A.3.from WO2010/069266.

The Metaflumizone analogue M.22C is described in CN 10171577. The phthalamides M.28.1 and M.28.2 are both known from WO 2007/101540. The anthranilamide M.28.3 has been described in WO2005/077934. The hydrazide compound M.28.4 has been described in WO 2007/043677. The anthranilamides M.28.5a) to M.28.5h) can be prepared as described in WO 2007/006670, WO2013/024009 and WO2013/024010, the anthranilamide M.28.5i) is described in WO2011/085575, the M.28.5j) in WO2008/134969, the M.28.5k) in US2011/046186 and the M.28.5l) in WO2012/034403. The diamide compounds M.28.6 and M.28.7 can be found in CN102613183.

The compounds M.UN.X.6a) to M.UN.X.6i) listed in M.UN.X.6 have been described in WO2012/029672. The mesoionic antagonist compound M.UN.X.7 was described in WO2012/092115, the nematicide M.UN.X.8 in WO2013/055584 and the Pyridalyl-type analogue M.UN.X.10 in WO2010/060379.

Preferred additional pesticidally active ingredients are those selected from the IRAC group 1, the Acetylcholinesterase (AChE) inhibitors, herein from the group 1A (Carbamtes) Thiodicarb, Methomyl and Carbaryl, and from the group 1B(Organophosphates), especially Acephate, Chlorpyriphos and Dimethoate, from the group 2B, the fiproles, here especially ethiprole and fipronil, from the group 3, the pyrethroids, here especially lambda-cyhalothrin, alpha-cypermethrin or deltametrin, and from the group 4A, the neonicotinoids, here especially acetamiprid, clothianidin, dinotefuran, imidacloprid, nitenpyram, thiacloprid or thiomethoxam.

Especially combinations of compounds of the invention with fiproles, neonictinoids or pyrethroids may possibly exhibit synergistic control of stinkbugs (according to the Colby formula), in particular Euschistus, e.g. Euschistus heros.

The following list F of active substances, in conjunction with which the compounds according to the invention can be used, is intended to illustrate the possible combinations but does not limit them:

F.I) Respiration inhibitors
F.I 1) Inhibitors of complex III at Qₒ site (e.g. strobilurins): azoxystrobin, coumethoxystrobin, coumoxystrobin, dimoxystrobin, enestroburin, fenaminstrobin, fenoxystrobin/flufenoxystrobin, fluoxastrobin, kresoxim-methyl, mandestrobine, metominostrobin, orysastrobin, picoxystrobin, pyraclostrobin, pyrametostrobin, pyraoxystrobin, trifloxystrobin and 2-(2-(3-(2,6-dichlorophenyl)-1-methylallylideneaminooxymethyl)-phenyl)-2-methoxyimino-N-methyl-acetamide, pyribencarb, triclopyricarb/chlorodincarb, famoxadone, fenamidone;
F.I 2) inhibitors of complex III at Qᵢ site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate; (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate;
F.I 3) inhibitors of complex II (e. g. carboxamides): benodanil, benzovindiflupyr, bixafen, boscalid, carboxin, fenfuram, fluopyram, flutolanil, fluxapyroxad, furametpyr, isofetamid, isopyrazam, mepronil, oxycarboxin, penflufen, penthiopyrad, sedaxane, tecloftalam, thifluzamide, N-(4′-trifluoromethylthiobiphenyl-2-yl)-3-difluoromethyl-1-methyl-1H-pyrazole-4-carboxamide, N-(2-(1,3,3-trimethyl-butyl)-phenyl)-1,3-dimethyl-5-fluoro-1H-pyrazole-4-carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethyl-indan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide, N-(7-fluoro-1,1,3-trimethyl-indan-4-yl)-1,3-dimethyl-pyrazole-4-carboxamide, N-[2-(2,4-dichlorophenyl)-2-methoxy-1-methyl-ethyl]-3-(difluoromethyl)-1-methyl-pyrazole-4-carboxamide, N-[2-(2,4-difluorophenyl)phenyl]-3-(trifluoromethyl)pyrazine-2-carboxamide;
F.I 4) other respiration inhibitors (e.g. complex I, uncouplers): diflumetorim, (5,8-difluoroquinazolin-4-yl)-{2-[2-fluoro-4-(4-trifluoromethylpyridin-2-yloxy)phenyl]-ethyl}-amine; nitrophenyl derivates: binapacryl, dinobuton, dinocap, fluazinam; ferimzone; organometal compounds: fentin salts, such as fentinacetate, fentin chloride or fentin hydroxide; ametoctradin; and silthiofam;
   F.II) Sterol biosynthesis inhibitors (SBI fungicides)
F.II 1) C14 demethylase inhibitors (DMI fungicides): triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-[rel-(2S;3R)-3-(2-chlorophenyl)-2-(2,4-difluorophenyl)-oxiranylmethyl]-5-thiocyanato-1H-[1,2,4]triazole, 2-[rel-(2S;3R)-3-(2-chlorophenyl)-2-(2,4-difluorophenyl)-oxiranylmethyl]-2H-[1,2,4]triazole-3-thiol, 2-[2-chloro-4-(4-chlorophenoxy)phenyl]-1-(1,2,4-triazol-1-yl)pentan-2-ol, 1-[4-(4-chlorophenoxy)-2-(trifluoromethyl)phenyl]-1-cyclopropyl-2-(1,2,4-triazol-1-yl)ethanol, 2-[4-(4-chlorophenoxy)-2-(trifluoromethyl)phenyl]-1-(1,2,4-triazol-1-yl)butan-2-ol, 2-[2-chloro-4-(4-chlorophenoxy)phenyl]-1-(1,2,4-triazol-1-yl)butan-2-ol, 2-[4-(4-chlorophenoxy)-2-(trifluoromethyl)phenyl]-3-methyl-1-(1,2,4-triazol-1-yl)butan-2-ol, 2-[4-(4-chlorophenoxy)-2-(trifluoromethyl)phenyl]-1-(1,2,4-triazol-1-yl)propan-2-ol, 2-[2-chloro-4-(4-chlorophenoxy)phenyl]-3-methyl-1-(1,2,4-triazol-1-yl)butan-2-ol, 2-[4-(4-chlorophenoxy)-2-(trifluoromethyl)phenyl]-1-(1,2,4-triazol-1-yl)pentan-2-ol, 2-[4-(4-fluorophenoxy)-2-(trifluoromethyl)phenyl]-1-(1,2,4-triazol-1-yl)propan-2-ol; imidazoles: imazalil, pefurazoate, prochloraz, triflumizol; pyrimidines, pyridines and piperazines: fenarimol, nuarimol, pyrifenox, triforine, [3-(4-chloro-2-fluorophenyl)-5-(2,4-difluorophenyl)isoxazol-4-yl]-(3-pyridyl)methanol;
F.II 2) Delta14-reductase inhibitors: aldimorph, dodemorph, dodemorphacetate, fenpropimorph, tridemorph, fenpropidin, piperalin, spiroxamine;
F.II 3) Inhibitors of 3-keto reductase: fenhexamid;
   F.III) Nucleic acid synthesis inhibitors
F.III 1) phenylamides or acyl amino acid fungicides: benalaxyl, benalaxyl-M, kiralaxyl, metalaxyl, metalaxyl-M (mefenoxam), ofurace, oxadixyl;
F.III 2) others: hymexazole, octhilinone, oxolinic acid, bupirimate, 5-fluorocytosine, 5-fluoro-2-(p-tolylmethoxy)pyrimidin-4-amine, 5-fluoro-2-(4-fluorophenylmethoxy)pyrimidin-4-amine;
   F.IV) Inhibitors of cell division and cytoskeleton
F.IV 1) tubulin inhibitors, such as benzimidazoles, thiophanates: benomyl, carbendazim, fuberidazole, thiabendazole, thiophanate-methyl; triazolopyrimidines: 5-chloro-7-(4-methylpiperidin-1-yl)-6-(2,4,6-trifluorophenyl)-[1,2,4]triazolo[1,5-a]pyrimidine;
F.IV 2) other cell division inhibitors: diethofencarb, ethaboxam, pencycuron, fluopicolide, zoxamide, metrafenone, pyriofenone;
   F.V) Inhibitors of amino acid and protein synthesis
F.V 1) methionine synthesis inhibitors (anilino-pyrimidines): cyprodinil, mepanipyrim, pyrimethanil;
F.V 2) protein synthesis inhibitors: blasticidin-S, kasugamycin, kasugamycin hydrochloride-hydrate, mildiomycin, streptomycin, oxytetracyclin, polyoxine, validamycin A;
   F.VI) Signal transduction inhibitors
F.VI 1) MAP/histidine kinase inhibitors: fluoroimid, iprodione, procymidone, vinclozolin, fenpiclonil, fludioxonil;
F.VI 2) G protein inhibitors: quinoxyfen;
   F.VII) Lipid and membrane synthesis inhibitors
F.VII 1) Phospholipid biosynthesis inhibitors: edifenphos, iprobenfos, pyrazophos, isoprothiolane;
F.VII 2) lipid peroxidation: dicloran, quintozene, tecnazene, tolclofos-methyl, biphenyl, chloroneb, etridiazole;
F.VII 3) phospholipid biosynthesis and cell wall deposition: dimethomorph, flumorph, mandipropamid, pyrimorph, benthiavalicarb, iprovalicarb, valifenalate and N-(1-(1-(4-cyano-phenyl)ethanesulfonyl)-but-2-yl) carbamic acid-(4-fluorophenyl) ester;
F.VII 4) compounds affecting cell membrane permeability and fatty acides: propamocarb, propamocarb-hydrochlorid;
F.VII 5) fatty acid amide hydrolase inhibitors: oxathiapiprolin;
   F.VIII) Inhibitors with Multi Site Action
F.VIII 1) inorganic active substances: Bordeaux mixture, copper acetate, copper hydroxide, copper oxychloride, basic copper sulfate, sulfur;
F.VIII 2) thio- and dithiocarbamates: ferbam, mancozeb, maneb, metam, metiram, propineb, thiram, zineb, ziram;
F.VIII 3) organochlorine compounds (e.g. phthalimides, sulfamides, chloronitriles): anilazine, chlorothalonil, captafol, captan, folpet, dichlofluanid, dichlorophen, hexachlorobenzene, pentachlorphenole and its salts, phthalide, tolylfluanid, N-(4-chloro-2-nitro-phenyl)-N-ethyl-4-methyl-benzenesulfonamide;
F.VIII 4) guanidines and others: guanidine, dodine, dodine free base, guazatine, guazatine-acetate, iminoctadine, iminoctadine-triacetate, iminoctadinetris(albesilate), dithianon, 2,6-dimethyl-1H,5H-[1,4]dithiino[2,3-c:5,6-c′]dipyrrole-1,3,5,7(2H,6H)-tetraone;
   F.IX) Cell wall synthesis inhibitors
F.IX 1) inhibitors of glucan synthesis: validamycin, polyoxin B;
F.IX 2) melanin synthesis inhibitors: pyroquilon, tricyclazole, carpropamid, dicyclomet, fenoxanil;
   F.X) Plant defence inducers
F.X 1) acibenzolar-S-methyl, probenazole, isotianil, tiadinil, prohexadionecalcium;
F.X 2) phosphonates: fosetyl, fosetyl-aluminum, phosphorous acid and its salts, 4-cyclopropyl-N-(2,4-dimethoxyphenyl)thiadiazole-5-carboxamide;
   F.XI) Unknown mode of action
bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothalisopropyl, oxathiapiprolin, picarbutrazox, tolprocarb, 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2, 5-dimethyl-phenyl)-N-ethyl-N-methyl formamidine, N′-(4-(4-fluoro-3-trifluoromethyl-phenoxy)-2,5-dimethyl-phenyl)N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, methoxy-acetic acid 6-tert-butyl-8-fluoro-2,3-dimethyl-quinolin-4-yl ester, 3-[5-(4-methylphenyl)-2,3-dimethyl-isoxazolidin-3-yl]-pyridine, 3-[5-(4-chloro-phenyl)-2,3-dimethyl-isoxazolidin-3-yl]-pyridine (pyrisoxazole), N-(6-methoxypyridin-3-yl) cyclopropanecarboxylic acid amide, 5-chloro-1-(4,6-dimethoxypyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide, ethyl (Z)-3-amino-2-cyano-3-phenyl-prop-2-enoate, pentyl N-[6-[[(Z)-[(1-methyltetrazol-5-yl)-phenyl-methylene]amino]oxymethyl]-2-pyridyl]carbamate, 2-[2-[(7,8-difluoro-2-methyl-3-quinolyl)oxy]-6-fluoro-phenyl]propan-2-ol, 2-[2-fluoro-6-[(8-fluoro-2-methyl-3-quinolyl)oxy]phenyl]propan-2-ol, 3-(5-fluoro-3,3,4,4-tetramethyl-3,4-dihydroisoquinolin-1-yl)quinoline, 3-(4,4-difluoro-3,3-dimethyl-3,4-dihydroisoquinolin-1-yl)quinoline, 3-(4,4,5-trifluoro-3,3-dimethyl-3,4-dihydroisoquinolin-1-yl)quinoline;

### F.XII) Biopesticides

F.XII 1) Microbial pesticides with fungicidal, bactericidal, viricidal and/or plant defense activator activity: Ampelomyces quisqualis, Aspergillus flavus, Aureobasidium pullulans, Bacillus amyloliquefaciens, B. mojavensis, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus polymyxa, Pantoea vagans, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperellum, T. atroviride, T. fertile, T. gamsii, T. harmatum, T. harzianum; mixture of T. harzianum and T. viride; mixture of T. polysporum and T. harzianum; T. stromaticum, T. virens (also named Gliocladium virens), T. viride, Typhula phacorrhiza, Ulocladium oudemansii, Verticillium dahlia, zucchini yellow mosaic virus (avirulent strain);
F.XII 2) Biochemical pesticides with fungicidal, bactericidal, viricidal and/or plant defense activator activity: chitosan (hydrolysate), harpin protein, laminarin, Menhaden fish oil, natamycin, Plum pox virus coat protein, potassium or sodium bicarbonate, Reynoutria sachlinensis extract, salicylic acid, tea tree oil;

The fungicidal active compounds mentioned above of groups F.I to F.XI, their preparation and their action against harmful fungi are generally known (cf., for example, http://www.hclrss.demon.co.uk/index.html).

The fungicides of chemical nature described by common names, their preparation and their activity against pests are known (cf.: http://www.alanwood.net/pesticides/); these pesticides are often commercially available.

The fungicides described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657, WO2012/168188, WO 2007/006670, WO 11/77514; WO13/047749, WO 10/069882, WO 13/047441, WO 03/16303, WO 09/90181, WO 13/007767, WO 13/010862, WO 13/024009 and WO 13/024010).

The biopesticides from group F.XII) of fungicides, their preparation and their pesticidal activity e.g. against harmful fungi or insects are known (e-Pesticide Manual V 5.2 (ISBN 978 1 901396 85 0) (2008-2011); http://www.epa.gov/opp00001/biopesticides/, see product lists therein; http://www.omri.org/omri-lists, see lists therein; Bio-Pesticides Database BPDB http://sitem.herts.ac.uk/aeru/bpdb/, see A to Z link therein). The biopesticides from group F.XII. may also have insecticidal, acaricidal, molluscidal, pheromone, nematicidal, plant stress reducing, plant growth regulator, plant growth promoting and/or yield enhancing activity. The biopesticides from group L3) and/or L4) may also have fungicidal, bactericidal, viricidal, plant defense activator, plant stress reducing, plant growth regulator, plant growth promoting and/or yield enhancing activity. The biopesticides from group F.XII may also have fungicidal, bactericidal, viricidal, plant defense activator, insecticidal, acaricidal, molluscidal, pheromone and/or nematicidal activity.

Many of these biopesticides are registered and/or are commercially available: aluminium silicate (Screen™ Duo from Certis LLC, USA), Agrobacterium radio-bacter K1026 (e.g. NoGall® from Becker Underwood Pty Ltd., Australia), A. radiobacter K84 (Nature 280, 697-699, 1979; e.g. GallTroll® from AG Biochem, Inc., C, USA), Ampelomyces quisqualis M-10 (e.g. AQ 10® from Intrachem Bio GmbH & Co. KG, Germany), Ascophyllum nodosum (Norwegian kelp, Brown kelp) extract or filtrate (e.g. ORKA GOLD from Becker Underwood, South Africa; or Goemar® from Laboratoires Goemar, France), Aspergillus flavus NRRL 21882 isolated from a peanut in Georgia in 1991 by the USDA, National Peanut Research Laboratory (e.g. in Afla-Guard® from Syngenta, CH), mixtures of Aureobasidium pullulans DSM14940 and DSM 14941 (e.g. blastospores in BlossomProtect® from bio-ferm GmbH, Germany), Azospirillum brasilense XOH (e.g. AZOS from Xtreme Gardening, USA or RTI Reforestation Technologies International; USA), Bacillus amyloliquefaciens FZB42 (e.g. in RhizoVital® 42 from AbiTEP GmbH, Berlin, Germany), B. amyloliquefaciens IN937a (J. Microbiol. Biotechnol. 17(2), 280-286, 2007; e.g. in BioYield® from Gustafson LLC, TX, USA), B. amyloliquefaciens IT-45 (CNCM I-3800) (e.g. Rhizocell C from ITHEC, France), B. amyloliquefaciens subsp. plantarum MB1600 (NRRL B-50595, deposited at United States Department of Agriculture) (e.g. Integral®, Subtilex® NG from Becker Underwood, USA), B. cereus CNCM I-1562 (U.S. Pat. No. 6,406,690), B. firmus CNCM I-1582 (WO 2009/126473, WO 2009/124707, U.S. Pat. No. 6,406,690; Votivo® from Bayer Crop Science LP, USA), B. pumilus GB34 (ATCC 700814; e.g. in YieldShield® from Gustafson LLC, TX, USA), and Bacillus pumilus KFP9F (NRRL B-50754) (e.g. in BAC-UP or FUSION-P from Becker Underwood South Africa), B. pumilus QST 2808 (NRRL B-30087) (e.g. Sonata® and Ballad® Plus from AgraQuest Inc., USA), B. subtilis GB03 (e.g. Kodiak® or BioYield® from Gustafson, Inc., USA; or Companion® from Growth Products, Ltd., White Plains, N.Y. 10603, USA), B. subtilis GB07 (Epic® from Gustafson, Inc., USA), B. subtilis QST-713 (NRRL B-21661 in Rhapsody®, Serenade® MAX and Serenade® ASO from AgraQuest Inc., USA), B. subtilis var. amylolique-faciens FZB24 (e.g. Taegro® from Novozyme Biologicals, Inc., USA), B. subtilis var. amyloliquefaciens D747 (e.g. Double Nickel 55 from Certis LLC, USA), B. thuringiensis ssp. aizawai ABTS-1857 (e.g. in XenTari® from BioFa AG, Miunsingen, Germany), B. t. ssp. aizawai SAN 401 l, ABG-6305 and ABG-6346, Bacillus t. ssp. israelensis AM65-52 (e.g. in VectoBac® from Valent BioSciences, IL, USA), Bacillus thuringiensis ssp. kurstaki SB4 (NRRL B-50753; e.g. Beta Pro® from Becker Underwood, South Africa), B. t. ssp. kurstaki ABTS-351 identical to HD-1 (ATCC SD-1275; e.g. in Dipel® DF from Valent BioSciences, IL, USA), B. t. ssp. kurstaki EG 2348 (e.g. in Lepinox® or Rapax® from CBC (Europe) S.r.l., Italy), B. t. ssp. tenebrionis DSM 2803 (EP 0 585 215 B1; identical to NRRL B-15939; Mycogen Corp.), B. t. ssp. tenebrionis NB-125 (DSM 5526; EP 0 585 215 B1; also referred to as SAN 418 I or ABG-6479; former production strain of Novo-Nordisk), B. t. ssp. tenebrionis NB-176 (or NB-176-1) a gamma-irridated, induced high-yielding mutant of strain NB-125 (DSM 5480; EP 585 215 B1; Novodor® from Valent BioSciences, Switzerland), Beauveria bassiana ATCC 74040 (e.g. in Naturalis® from CBC (Europe) S.r.l., Italy), B. bassiana DSM 12256 (US 200020031495; e.g. BioExpert® SC from Live Sytems Technology S.A., Colombia), B. bassiana GHA (BotaniGard® 22WGP from Laverlam Int. Corp., USA), B. bassiana PPRI 5339 (ARSEF number 5339 in the USDA ARS collection of entomopathogenic fungal cultures; NRRL 50757) (e.g. BroadBand® from Becker Underwood, South Africa), B. brongniartii (e.g. in Melocont® from Agrifutur, Agrianello, Italy, for control of cockchafer; J. Appl. Microbiol. 100(5),1063-72, 2006), Bradyrhizobium sp. (e.g. Vault® from Becker Underwood, USA), B. japonicum (e.g. VAULT® from Becker Underwood, USA), Candida oleophila I-182 (NRRL Y-18846; e.g. Aspire® from Ecogen Inc., USA, Phytoparasitica 23(3), 231-234, 1995), C. oleophila strain O (NRRL Y-2317; Biological Control 51, 403-408, 2009), Candida saitoana (e.g. Biocure® (in mixture with lysozyme) and BioCoat® from Micro Flo Company, USA (BASF SE) and Arysta), Chitosan (e.g. Armour-Zen® from BotriZen Ltd., NZ), Clonostachys rosea f. catenulata, also named Gliocladium catenulatum (e.g. isolate J 1446: Prestop® from Verdera Oy, Finland), Chromobacterium subtsugae PRAA4-1 isolated from soil under an eastern hemlock (Tsuga canadensis) in the Catoctin Mountain region of central Maryland (e.g. in GRANDEVO from Marrone Bio Innovations, USA), Coniothyrium minitans CON/M/91-08 (e.g. Contans® WG from Prophyta, Germany), Cryphonectria parasitica (e.g. Endothia parasitica from CNICM, France), Cryptococcus albidus (e.g. YIELD PLUS® from Anchor Bio-Technologies, South Africa), Cryptophlebia leucotreta granulovirus (CrleGV) (e.g. in CRYPTEX from Adermatt Biocontrol, Switzerland), Cydia pomonella granulovirus (CpGV) V03 (DSM GV-0006; e.g. in MADEX Max from Andermatt Biocontrol, Switzerland), CpGV V22 (DSM GV-0014; e.g. in MADEX Twin from Adermatt Biocontrol, Switzerland), Delftia acidovorans RAY209 (ATCC PTA-4249; WO 2003/57861; e.g. in BIOBOOST from Brett Young, Winnipeg, Canada), Dilophosphora alopecuri (Twist Fungus from Becker Underwood, Australia), Ecklonia maxima (kelp) extract (e.g. KELPAK SL from Kelp Products Ltd, South Africa), formononetin (e.g. in MYCONATE from Plant Health Care plc, U.K.), Fusarium oxysporum (e.g. BIOFOX® from S.I.A.P.A., Italy, FUSACLEAN® from Natural Plant Protection, France), Glomus intraradices (e.g. MYC 4000 from ITHEC, France), Glomus intraradices RTI-801 (e.g. MYKOS from Xtreme Gardening, USA or RTI Reforestation Technologies International; USA), grapefruit seeds and pulp extract (e.g. BC-1000 from Chemie S.A., Chile), harpin (alpha-beta) protein (e.g. MESSENGER or HARP-N-Tek from Plant Health Care plc, U.K.; Science 257, 1-132, 1992), Heterorhabditis bacteriophaga (e.g. Nemasys® G from Becker Underwood Ltd., UK), Isaria fumosorosea Apopka-97 (ATCC 20874) (PFR-97™ from Certis LLC, USA), cis-jasmone (U.S. Pat. No. 8,221,736), laminarin (e.g. in VACCIPLANT from Laboratoires Goemar, St. Malo, France or Stähler SA, Switzerland), Lecanicillium longisporum KV42 and KV71 (e.g. VERTALEC® from Koppert BV, Netherlands), L. muscarium KV01 (formerly Verticillium lecanii) (e.g. MYCOTAL from Koppert BV, Netherlands), Lysobacter antibioticus 13-1 (Biological Control 45, 288-296, 2008), L. antibioticus HS124 (Curr. Microbiol. 59(6), 608-615, 2009), L. enzymogenes 3.1T8 (Microbiol. Res. 158, 107-115; Biological Control 31(2), 145-154, 2004), Metarhizium anisopliae var. acridum IMI 330189 (isolated from Ornithacris cavroisi in Niger; also NRRL 50758) (e.g. GREEN MUSCLE® from Becker Underwood, South Africa), M. a. var. acridum FI-985 (e.g. GREEN GUARD® SC from Becker Underwood Pty Ltd, Australia), M. anisopliae FI-1045 (e.g. BIOCANE® from Becker Underwood Pty Ltd, Australia), M. anisopliae F52 (DSM 3884, ATCC 90448; e.g. MET52® Novozymes Biologicals BioAg Group, Canada), M. anisopliae ICIPE 69 (e.g. METATHRIPOL from ICIPE, Nairobe, Kenya), Metschnikowia fructicola (NRRL Y-30752; e.g. SHEMER® from Agrogreen, Israel, now distributed by Bayer CropSciences, Germany; U.S. Pat. No. 6,994,849), Microdochium dimerum (e.g. ANTIBOT® from Agrauxine, France), Microsphaeropsis ochracea P130A (ATCC 74412 isolated from apple leaves from an abandoned orchard, St-Joseph-du-Lac, Quebec, Canada in 1993; Mycologia 94(2), 297-301, 2002), Muscodor albus QST 20799 originally isolated from the bark of a cinnamon tree in Honduras (e.g. in development products Muscudor™ or QRD300 from AgraQuest, USA), Neem oil (e.g. TRILOGY®, TRIACT® 70 EC from Certis LLC, USA), Nomuraea rileyi strains SA86101, GU87401, SR86151, CG128 and VA9101, Paecilomyces fumosoroseus FE 9901 (e.g. NO FLY™ from Natural Industries, Inc., USA), P. lilacinus 251 (e.g. in BioAct®/MeloCon® from Prophyta, Germany; Crop Protection 27, 352-361, 2008; originally isolated from infected nematode eggs in the Philippines), P. lilacinus DSM 15169 (e.g. NEMATA® SC from Live Systems Technology S.A., Colombia), P. lilacinus BCP2 (NRRL 50756; e.g. PL GOLD from Becker Underwood BioAg SA Ltd, South Africa), mixture of Paenibacillus alvei NAS6G6 (NRRL B-50755), Pantoea vagans (formerly agglomerans) C9-1 (originally isolated in 1994 from apple stem tissue; BlightBan C9-1® from NuFrams America Inc., USA, for control of fire blight in apple; J. Bacteriol. 192(24) 6486-6487, 2010), Pasteuria spp. ATCC PTA-9643 (WO 2010/085795), Pasteuria spp. ATCC SD-5832 (WO 2012/064527), P. nishizawae (WO 2010/80169), P. penetrans (U.S. Pat. No. 5,248,500), P. ramose (WO 2010/80619), P. thornea (WO 2010/80169), P. usgae (WO 2010/80169), Penicillium bilaiae (e.g. Jump Start® from Novozymes Biologicals BioAg Group, Canada, originally isolated from soil in southern Alberta; Fertilizer Res. 39, 97-103, 1994), Phlebiopsis gigantea (e.g. RotStop® from Verdera Oy, Finland), Pichia anomala WRL-076 (NRRL Y-30842; U.S. Pat. No. 8,206,972), potassium bicarbonate (e.g. Amicarb® fromm Stähler SA, Switzerland), potassium silicate (e.g. Sil-MATRIX™ from Certis LLC, USA), Pseudozyma flocculosa PF-A22 UL (e.g. Sporodex® from Plant Products Co. Ltd., Canada), Pseudomonas sp. DSM 13134 (WO 2001/40441, e.g. in PRORADIX from Sourcon Padena GmbH & Co. K G, Hechinger Str. 262, 72072 Tubingen, Germany), P. chloraphis MA 342 (e.g. in CERALL or CEDEMON from BioAgri AB, Uppsala, Sweden), P. fluorescens CL 145A (e.g. in ZEQUANOX from Marrone Biolnnovations, Davis, Calif., USA; J. Invertebr. Pathol. 113(1):104-14, 2013), Pythium oligandrum DV 74 (ATCC 38472; e.g. POLYVERSUM® from Remeslo SSRO, Biopreparaty, Czech Rep. and GOWAN, USA; US 2013/0035230), Reynoutria sachlinensis extract (e.g. REGALIA® SC from Marrone Biolnnovations, Davis, Calif., USA), Rhizobium leguminosarum bv. phaseolii (e.g. RHIZO-STICK from Becker Underwood, USA), R. l. trifolii RP113-7 (e.g. DORMAL from Becker Underwood, USA; Appl. Environ. Microbiol. 44(5), 1096-1101), R. l. bv. viciae P1NP3Cst (also referred to as 1435; New Phytol 179(1), 224-235, 2008; e.g. in NODULATOR PL Peat Granule from Becker Underwood, USA; or in NODULATOR XL PL bfrom Becker Underwood, Canada), R. l. bv. viciae SU303 (e.g. NODULAID Group E from Becker Underwood, Australia), R. l. bv. viciae WSM1455 (e.g. NODULAID Group F from Becker Underwood, Australia), R. tropici SEMIA 4080 (identical to PRF 81; Soil Biology & Biochemistry 39, 867-876, 2007), Sinorhizobium meliloti MSDJ0848 (INRA, France) also referred to as strain 2011 or RCR2011 (Mol Gen Genomics (2004) 272: 1-17; e.g. DORMAL ALFALFA from Becker Underwood, USA; NITRAGIN® Gold from Novozymes Biologicals BioAg Group, Canada), Sphaerodes mycoparasitica IDAC 301008-01 (WO 2011/022809), Steinernema carpocapsae (e.g. MILLENIUM® from Becker Underwood Ltd., UK), S. feltiae (NEMASHIELD® from BioWorks, Inc., USA; NEMASYS® from Becker Underwood Ltd., UK), S. kraussei L137 (NEMASYS® L from Becker Underwood Ltd., UK), Streptomyces griseoviridis K61 (e.g. MYCOSTOP® from Verdera Oy, Espoo, Finland; Crop Protection 25, 468-475, 2006), S. lydicus WYEC 108 (e.g. Actinovate® from Natural Industries, Inc., USA, U.S. Pat. No. 5,403,584), S. violaceusniger YCED-9 (e.g. DT-9® from Natural Industries, Inc., USA, U.S. Pat. No. 5,968,503), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. asperellum ICC 012 (e.g. in TENET WP, REMDIER WP, BIOTEN WP from Isagro NC, USA, BIO-TAM from AgraQuest, USA), T. atroviride LC52 (e.g. SENTINEL® from Agrimm Technologies Ltd, NZ), T. atroviride CNCM I-1237 (e.g. in Esquive WG from Agrauxine S.A., France, e.g. against pruning wound diseases on vine and plant root pathogens), T. fertile JM41R (NRRL 50759; e.g. RICHPLUS™ from Becker Underwood Bio Ag SA Ltd, South Africa), T. gamsii ICC 080 (e.g. in TENET WP, REMDIER WP, BIOTEN WP from Isagro NC, USA, BIO-TAM from AgraQuest, USA), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB Bio-lnnovation AB, Sweden), T. stromaticum (e.g. TRICOVAB® from C.E.P.L.A.C., Brazil), T. virens GL-21 (also named Gliocladium virens) (e.g. SOILGARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy) and Ulocladium oudemansii HRU3 (e.g. in BOTRY-ZEN® from Botry-Zen Ltd, NZ).

Strains can be sourced from genetic resource and deposition centers: American Type Culture Collection, 10801 University Blvd., Manassas, Va. 20110-2209, USA (strains with ATCC prefic); CABI Europe—International Mycological Institute, Bakeham Lane, Egham, Surrey, TW20 9TYNRRL, UK (strains with prefices CABI and IMI); Centraalbureau voor Schimmelcultures, Fungal Biodiversity Centre, Uppsalaan 8, PO Box 85167, 3508 AD Utrecht, Netherlands (strains with prefic CBS); Division of Plant Industry, CSIRO, Canberra, Australia (strains with prefix CC); Collection Nationale de Cultures de Microorganismes, Institut Pasteur, 25 rue du Docteur Roux, F-75724 PARIS Cedex 15 (strains with prefix CNCM); Leibniz-lnstitut DSMZ-Deutsche Sammlung von Mikroorganismen und Zellkulturen GmbH, Inhoffenstrale 7 B, 38124 Braunschweig, Germany (strains with prefix DSM); International Depositary Authority of Canada Collection, Canada (strains with prefix IDAC); Interntional Collection of Microorgniasms from Plants, Landcare Research, Private Bag 92170, Auckland Mail Centre, Auckland 1142, New Zealand (strans with prefix ICMP); IITA, PMB 5320, Ibadan, Nigeria (straisn with prefix IITA); The National Collections of Industrial and Marine Bacteria Ltd., Torry Research Station, P.O. Box 31, 135 Abbey Road, Aberdeen, AB9 8DG, Scotland (strains with prefix NCIMB); ARS Culture Collection of the National Center for Agricultural Utilization Research, Agricultural Research Service, U.S. Department of Agriculture, 1815 North University Street, Peoria, Ill. 61604, USA (strains with prefix NRRL); Department of Scientific and Industrial Research Culture Collection, Applied Biochemistry Division, Palmerston North, New Zealand (strains with prefix NZP); FEPAGRO-Fundação Estadual de Pesquisa Agropecuária, Rua Gonralves Dias, 570, Bairro Menino Deus, Porto Alegre/RS, Brazil (strains with prefix SEMIA); SARDI, Adelaide, South Australia (strains with prefix SRDI); U.S. Department of Agriculture, Agricultural Research Service, Soybean and Alfalfa Research Laboratory, BARC-West, 10300 Baltimore Boulevard, Building 011, Room 19-9, Beltsville, Md. 20705, USA (strains with prefix USDA: Beltsville Rhizobium Culture Collection Catalog March 1987 USDA-ARS ARS-30: http://pdf.usaid.gov/pdf_docs/PNAAW891.pdf); and Murdoch University, Perth, Western Australia (strains with prefix WSM). Further strains may be found at the Global catalogue of Microorganisms: http://gcm.wfcc.info/ and http://www.landcareresearch.co.nz/resources/collections/icmp and further references to strain collections and their prefixes at http://refs.wdcm.org/collections.htm. Bacillus amyloliquefaciens subsp. plantarum MBI600 (NRRL B-50595) is deposited under accession number NRRL B-50595 with the strain designation Bacillus subtilis 1430 (and identical to NCIMB 1237). Recently, MBI 600 has been re-classified as Bacillus amyloliquefaciens subsp. plantarum based on polyphasic testing which combines classical microbiological methods relying on a mixture of traditional tools (such as culture-based methods) and molecular tools (such as genotyping and fatty acids analysis). Thus, Bacillus subtilis MBI600 (or MBI 600 or MBI-600) is identical to Bacillus amyloliquefaciens subsp. plantarum MBI600, formerly Bacillus subtilis MBI600. Bacillus amyloliquefaciens MBI600 is known as plant growth-promoting rice seed treatment from Int. J. Microbiol. Res. 3(2) (2011), 120-130 and further described e.g. in US 2012/0149571 A1. This strain MB1600 is e.g. commercially available as liquid formulation product INTEGRAL® (Becker-Underwood Inc., USA).

Bacillus subtilis strain FB17 was originally isolated from red beet roots in North America (System Appl. Microbiol 27 (2004) 372-379). This B. subtilis strain promotes plant health (US 2010/0260735 A1; WO 2011/109395 A2). B. subtilis FB17 has also been deposited at ATCC under number PTA-11857 on Apr. 26, 2011. Bacillus subtilis strain FB17 may be referred elsewhere to as UD1022 or UD10-22.

Bacillus amyloliquefaciens AP-136 (NRRL B-50614), B. amyloliquefaciens AP-188 (NRRL B-50615), B. amyloliquefaciens AP-218 (NRRL B-50618), B. amyloliquefaciens AP-219 (NRRL B-50619), B. amyloliquefaciens AP-295 (NRRL B-50620), B. japonicum SEMIA 5079 (e.g. Gelfix 5 or Adhere 60 from Nitral Urbana Laoboratories, Brazil, a BASF Company), B. japonicum SEMIA 5080 (e.g. GELFIX 5 or ADHERE 60 from Nitral Urbana Laoboratories, Brazil, a BASF Company), B. mojavensis AP-209 (NRRL B50616), B. solisalsi AP-217 (NRRL B-50617), B. pumilus strain INR-7 (otherwise referred to as BU-F22 (NRRL B-50153) and BU-F33 (NRRL B-50185)), B. simplex ABU 288 (NRRL B-50340) and B. amyloliquefaciens subsp. plantarum MBI600 (NRRL B50595) have been mentioned i.a. in US patent appl. 20120149571, U.S. Pat. No. 8,445,255, WO 2012/079073. Bradyrhizobium japonicum USDA 3 is known from U.S. Pat. No. 7,262,151. Jasmonic acid or salts (jasmonates) or derivatives include without limitation potassi-um jasmonate, sodium jasmonate, lithium jasmonate, ammonium jasmonate, dimethyl-ammonium jasmonate, isopropylammonium jasmonate, diolammonium jasmonate, diethtriethanolammonium jasmonate, jasmonic acid methyl ester, jasmonic acid amide, jasmonic acid methylamide, jasmonic acid-L-amino acid (amide-linked) conjugates (e.g., conjugates with L-isoleucine, L-valine, L-leucine, or L-phenylalanine), 12-oxo-phytodienoic acid, coronatine, coronafacoyl-L-serine, coronafacoyl-L-threonine, methyl esters of 1-oxo-indanoyl-isoleucine, methyl esters of 1-oxo-indanoyl-leucine, coronalon (2-[(6-ethyl-1-oxo-indane-4-carbonyl)-amino]-3-methyl-pentanoic acid methyl ester), linoleic acid or derivatives thereof and cis-jasmone, or combinations of any of the above.

Humates are humic and fulvic acids extracted from a form of lignite coal and clay, known as leonardite. Humic acids are organic acids that occur in humus and other organically derived materials such as peat and certain soft coal. They have been shown to increase fertilizer efficiency in phosphate and micro-nutrient uptake by plants as well as aiding in the development of plant root systems.

The compounds of the invention may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same

The invertebrate pest (also referred to as “animal pest”), i.e. the insects, arachnids and nematodes, the plant, soil or water in which the plant is growing or may grow can be contacted with the compounds of the present invention or composition(s) comprising them by any application method known in the art. As such, “contacting” includes both direct contact (applying the compounds/compositions directly on the invertebrate pest or plant—typically to the foliage, stem or roots of the plant) and indirect contact (applying the compounds/compositions to the locus of the invertebrate pest or plant).

The compounds of the present invention or the pesticidal compositions comprising them may be used to protect growing plants and crops from attack or infestation by animal pests, especially insects, acaridae or arachnids by contacting the plant/crop with a pesticidally effective amount of compounds of the present invention. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with an insecticidally effective amount of the active compounds. The application can be carried out both before and after the infection of the plants, plant propagation materials, such as seeds, soil, surfaces, materials or rooms by the insects.

Moreover, invertebrate pests may be controlled by contacting the target pest, its food supply, habitat, breeding ground or its locus with a pesticidally effective amount of compounds of the present invention. As such, the application may be carried out before or after the infection of the locus, growing crops, or harvested crops by the pest.

The compounds of the present invention can also be applied preventively to places at which occurrence of the pests is expected.

The compounds of the present invention may be also used to protect growing plants from attack or infestation by pests by contacting the plant with a pesticidally effective amount of compounds of the present invention. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 5 g to 500 g per hectare, more desirably from 5 g to 200 g per hectare.

The compounds of the present invention are effective through both contact (via soil, glass, wall, bed net, carpet, plant parts or animal parts), and ingestion (bait, or plant part).

The compounds of the present invention may also be applied against non-crop insect pests, such as ants, termites, wasps, flies, mosquitos, crickets, or cockroaches. For use against said non-crop pests, compounds of the present invention are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics. The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active ingredient.

Formulations of compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets. The compounds of the present invention and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of the present invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of the present invention are also suitable for the treatment of plant propagation material, especially seeds, in order to protect them from insect pest, in particular from soil-living insect pests and the resulting plant's roots and shoots against soil pests and foliar insects.

The compounds of the present invention are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the present invention, including a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A 242 236, EP-A 242 246) (WO 92/00377) (EP-A 257 993, U.S. Pat. No. 5,013,659) or in transgenic crop plants, for example cotton, with the capability of producing Bacillus thuringiensis toxins (Bt toxins) which make the plants resistant to certain pests (EP-A 142 924, EP-A 193 259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of the present invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds of the present invention are generally from 0.01 g to 10 kg per 100 kg of seed, preferably from 0.05 g to 5 kg per 100 kg of seed, more preferably from 0.1 g to 1000 g per 100 kg of seed and in particular from 0.1 g to 200 g per 100 kg of seed.

The invention therefore also relates to seed comprising a compound of the present invention, including an agriculturally useful salt of it, as defined herein. The amount of the compound of the present invention, including an agriculturally useful salt thereof will in general vary from 0.01 g to 10 kg per 100 kg of seed, preferably from 0.05 g to 5 kg per 100 kg of seed, in particular from 0.1 g to 1000 g per 100 kg of seed. For specific crops such as lettuce the rate can be higher.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of the present invention into contact with each other, and “seed dressing” to methods of seed treatment which provide the seeds with an amount of the compounds of the present invention, i.e. which generate a seed comprising a compound of the present invention. In principle, the treatment can be applied to the seed at any time from the harvest of the seed to the sowing of the seed. The seed can be treated immediately before, or during, the planting of the seed, for example using the “planter's box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of the present invention, including their stereoisomers, veterinarily acceptable salts or N-oxides, are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions comprising a parasiticidally effective amount of compounds of the present invention, including their stereoisomers, veterinarily acceptable salts or N-oxides, and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of the present invention, including its stereoisomers, veterinarily acceptable salts or N-oxides, or a composition comprising it.

The invention also provides the use of a compound of the present invention, including its stereoisomers, veterinarily acceptable salts or N-oxides, for treating or protecting an animal from infestation or infection by invertebrate pests.

The invention also provides a process for the preparation of a composition for treating, controlling, preventing or protecting animals against infestation or infection by parasites which comprises a parasiticidally effective amount of a compound of the present invention, including its stereoisomers, veterinarily acceptable salts or N-oxides, or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula (I) and their stereoisomers, veterinarily acceptable salts, tautomers and N-oxides, are suitable for combating endo- and ectoparasites in and on animals.

The compounds of the present invention, especially compounds of formula (I) and their stereoisomers, veterinarily acceptable salts, tautomers and N-oxides, and compositions comprising them are preferably used for controlling and preventing infestations of and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels. Compounds of the present invention, including their stereoisomers, veterinarily acceptable salts or N-oxides, and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention, including their stereoisomers, veterinarily acceptable salts or N-oxides, and compositions comprising them are suitable for systemic and/or non-systemic control of ecto- and/or endoparasites. They are active against all or some stages of development.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus, cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp.,
Bugs (Heteropterida): Cimex lectularius, Cimex hemipterus, Reduvius senilis, Triatoma spp., Rhodnius ssp., Panstrongylus ssp. and Arilus critatus,
Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp, Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp.,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp., Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The present invention relates to the therapeutic and the non-therapeutic use of compounds of the present invention and compositions comprising them for controlling and/or combating parasites in and/or on animals. The compounds of the present invention and compositions comprising them may be used to protect the animals from attack or infestation by parasites by contacting them with a parasiticidally effective amount of compounds of the present invention and compositions containing them.

The compounds of the present invention and compositions comprising them can be effective through both contact (via soil, glass, wall, bed net, carpet, blankets or animal parts) and ingestion (e.g. baits). As such, “contacting” includes both direct contact (applying the pesticidal mixtures/compositions containing the compounds of the present invention directly on the parasite, which may include an indirect contact at its locus-P, and optionally also administrating the pesticidal mixtures/composition directly on the animal to be protected) and indirect contact (applying the compounds/compositions to the locus of the parasite). The contact of the parasite through application to its locus is an example of a non-therapeutic use of compounds of the present invention. “Locus-P” as used above means the habitat, food supply, breeding ground, area, material or environment in which a parasite is growing or may grow outside of the animal.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions of the present invention. A parasiticidally effective amount of the compositions will also vary according to the prevailing conditions such as desired parasiticidal effect and duration, target species, mode of application, and the like.

The compounds of the present invention can also be applied preventively to places at which occurrence of the pests or parasites are expected.

Administration can be carried out both prophylactically and therapeutically.

Administration of the active compounds is carried out directly or in the form of suitable preparations, orally, topically/dermally or parenterally.

### EXAMPLES

The present invention is now illustrated in further details by the following examples, without imposing any limitation thereto.

### Preparation Examples

Compounds can be characterized e.g. by coupled High Performance Liquid Chromatography/mass spectrometry (HPLC/MS), by ¹H-NMR and/or by their melting points.

Analytical HPLC column:

Method A: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.20 min; flow: 0.8-1.0 mL/min in 1.50 minutes at 60° C.

Method B: Agilent Eclipse Plus C18, 150 mm×4.6 mm ID, 5 μm: mobile phase: A: 0.1% TFA in water. B: 0.1% TFA in acetonitrile. Gradient:10% B-80% B-5 min, hold for 2 min, 3 min-0.10% B. Flow: 1.4 mL/min. Column temp: 30° C. Run time: 10 min.

Method C: Analytical UPLC column Aquity BEH C18, 1.7 μm, 2.1×50 m; mobile phase A: 0.05% formic acid in water, B: 0.05% formic acid in acetonitrile. Gradient: time/A %: 0/97, 0.3/97, 3.5/2, 4.8/2, 5/97, 5.01/97; flow: 0.6 mL/min; temp: 35° C.

Method D: X Bridge C18 (100 mm×4.6 mm) 3.5 μm; column temp. 35° C.; mobile phase A: 10 mM ammonium acetate; B: acetonitrile; gradient Time/B %: 0/5, 1/5, 2/15, 5/98, 7, 98, 8/5, 10/5; flow rate: 1 mL/min

Method E: Agilent Eclipse Plus C18, 50 mm×4.6 mm D, 5 μm; mobile phase: A: 10 mM ammonium formate (0.1 formic acid), B: acetonitrile (0.1 formic acid). Gradient:10% B-100% B-3 min, hold for 1 min, 1 min-10% B. Flow: 1.2 mL/min Column temp: 30° C. Run time: 5.01 min.

MS-method: ESI positive.

¹H-NMR: The signals are characterized by chemical shift (ppm, δ [delta]) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplett, q=quartett, t=triplett, d=doublet and s=singulett.

Abbreviations used are: d for day(s), h for hour(s), min for minute(s), r.t./room temperature for 20-25° C., THF for tetrahydrofuran, DMF for dimethylformamide, OAc for acetate, HATU for O-(7-Azabenzotriazol-1-yl)-N,N,N′,N′-tetramethyluronium hexafluorophosphate.

### A. Synthesis Examples

### Example P.1

### Ethyl-2-[(3,4-dichlorophenyl)hydrazono]propanoate

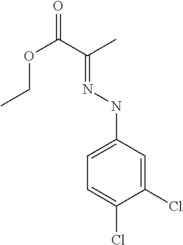 [SMILES: CCOC(=O)/C(C)=N/Nc1ccc(Cl)c(Cl)c1]

A mixture of (3,4-dichlorophenyl)hydrazine hydrochloride (4.3 g, 20 mmol, 1 equiv.), ethyl 2-oxopropanoate (3.74 g, 3.65 mL, 32.2 mmol, 1.6 equiv.), glacial acetic acid (0.25 mL, 0.24 g, 4.0 mmol, 1.05 equiv.) and ethanol (50 mL) were heated at reflux for 5 h, cooled and concentrated in vacuum. The residue was taken up in a minimal amount of ether and stirred under ice-cooling. The resulting solid was collected by filtration and yielded the title compound (3.64 g, 66%) as a solid.

¹H-NMR (400 MHz, CDCl₃): δ [delta]=1.40 (t, 3H), 2.12 (s, 3H), 4.34 (q, 2H), 7.03 (dd, 1H), 7.35 (m, 2H), 7.65 (br. s, 1H).

### Example P.2

### Ethyl-1-(3,4-dichlorophenyl)-4-formyl-pyrazole-3-carboxylate

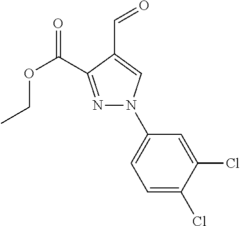 [SMILES: CCOC(=O)c1nn(-c2ccc(Cl)c(Cl)c2)cc1C=O]

To a cooled portion of DMF (2.04 mL, 1.93 g, 26.5 mmol, 2.0 equiv.) was added POCl₃ (3.69 mL, 6.09 g, 45 mmol, 3.0 equiv.) at 0° C. The mixture solidified and was diluted with a minimal amount of DMF. At this temperature, ethyl-2-[(3,4-dichlorophenyl)hydrazono]propanoate (obtained in Example P.1, 3.64 g, 13.2 mmol, 1.00 equiv.) in DMF (10 mL) was added and the mixture was allowed to warm to room temperature. After 16 h, the mixture was poured onto diluted sodium bicarbonate solution, cooled to 0° C. and brought to pH 4 with 2 N NaOH. The resulting precipitate was collected by filtration and dried in vacuum to obtain the title compound (3.5 g, 85%) as a solid.

¹H-NMR (400 MHz, CDCl₃): δ [delta]=1.47 (t, 3H), 4.52 (q, 2H), 7.60 (s, 2H), 7.98 (m, 1H), 8.44 (s, 1H), 10.43 (s, 1H).

### Example P.3

### Ethyl-4-[(4-cyano-1-piperidyl)methyl]-1-(3,4-dichlorophenyl)pyrazole-3-carboxylate

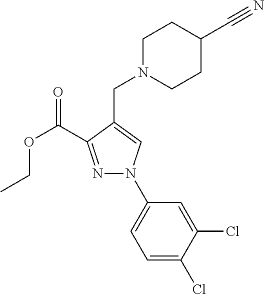 [SMILES: CCOC(=O)c1nn(-c2ccc(Cl)c(Cl)c2)cc1CN1CCC(C#N)CC1]

To a solution of ethyl-1-(3,4-dichlorophenyl)-4-formyl-pyrazole-3-carboxylate (obtained in Example P.2, 1.00 g, 3.19 mmol, 1.00 equiv.) and 4-cyanopiperidine (0.35 g. 3.19 mmol, 1.00 equiv.) in dichloromethane (20 mL) were added 2 drops of glacial acetic acid. After 30 min at room temperature, sodium triacetoxy borohydride (1.02 g, 4.79 mmol. 1-50 equiv.) was added and the mixture was stirred for 1.5 h. Saturated sodium bicarbonate solution was added and the layers were separated. The aqueous layer was extracted with dichloromethane and combined organic layers were dried over sodium sulfate and concentrated in vacuum to obtain the title compound (1.03 g, 79%).

¹H-NMR (400 MHz, CDCl₃): δ [delta]=1.45 (t, 3H), 1.82-2.06 (m, 4H). 2.41 (m, 2H), 2.64 (m, 1H), 2.88 (m, 2H), 3.80 (s, 2H), 4.43 (q, 2H), 7.54 (m, 1H), 7.61 (m, 1H), 7.91 (m, 2H).

### Example P.4

### 1-[[1-(3,4-Dichlorophenyl)-3-(hydroxymethyl)pyrazol-4-yl]methyl]piperidine-4-carbonitrile

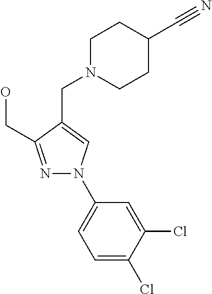 [SMILES: N#CC1CCN(Cc2cn(-c3ccc(Cl)c(Cl)c3)nc2CO)CC1]

To a solution of ethyl-4-[(4-cyano-1-piperidyl)methyl]-1-(3,4-dichlorophenyl)pyrazole-3-carboxylate (obtained in Example P.3, 1.03 g, 2.53 mmol, 1.00 equiv.) in tetrahydrofuran (10 mL) and ethanol (10 mL) was added a solution of lithium borohydride (5.06 mL of a 2 M solution in THF, 10.1 mmol, 4.0 equiv.) at 0° C. The reaction was allowed to reach room temperature and quenched with water after 5 h. The resulting mixture was extracted with ethyl acetate and combined organic layers were dried over sodium sulfate and evaporated to obtain the title compound (0.65 g, 70%).

HPLC-MS: 0.835 min, m/z=365.6

### Example P.5

### 1-[[1-(3,4-Dichlorophenyl)-3-formyl-pyrazol-4-yl]methyl]piperidine-4-carbonitrile

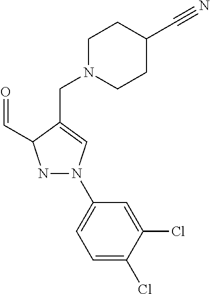 [SMILES: N#CC1CCN(CC2=CN(c3ccc(Cl)c(Cl)c3)NC2C=O)CC1]

To a solution of 1-[[1-(3,4-dichlorophenyl)-3-(hydroxymethyl)pyrazol-4-yl]methyl]-piperidine-4-carbonitrile (0.65 g, 1.8 mmol, 1.0 equiv.) in dichloromethane (20 mL) was added Dess-Martin-periodinane (0.83 g, 2.0 mmol, 1.10 equiv.) and stirred at room temperature for 1.5 h. After completion of the reaction, saturated sodium bicarbonate solution and sodium dithionate solution were added. The layers were separated and the organic layer was dried over sodium sulfate and concentrated in vacuum to obtain the title compound (0.67 g, quant.) which was used in the next step without further purification.

¹H-NMR (400 MHz, CDCl₃, containing impurities in the aliphatic and aromatic region): δ [delta]=1.80-2.10 (m, 4H). 2.42 (m, 2H), 2.66 (m, 1H), 2.82 (m, 2H), 3.83 (m, 2H), 7.56-7.68 (m, 2H), 7.83 (m, 2H), 10.12 (s, 1H).

### Example P.6

### 1-[[1-(3,4-dichlorophenyl)-3-[(E/Z)-methoxyiminomethyl]pyrazol-4-yl]methyl]piperidine-4-carbonitrile (Compound IA-1)

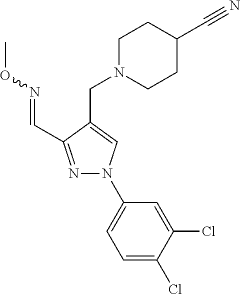 [SMILES: CON=Cc1nn(-c2ccc(Cl)c(Cl)c2)cc1CN1CCC(C#N)CC1]

A solution of 1-[[1-(3,4-dichlorophenyl)-3-formyl-pyrazol-4-yl]methyl]piperidine-4-carbonitrile (obtained in Example P.5, 0.34 g, 0.94 mmol, 1.0 equiv.), methylhydroxylamine hydrochloride (86 mg, 1.03 mmol, 1.1 equiv.) and sodium acetate (84 mg, 1.03 mmol, 1.1 equiv) in ethanol (10 mL) was heated at reflux for 3 h. after cooling, water was added and the mixture was extracted with dichloromethane. Combined organic layers were dried over sodium sulfate and concentrated in vacuum. The residue was purified via column chromatography on silica gel to obtain the title compound (0.18 g, 44%).

¹H-NMR (400 MHz, CDCl₃): δ [delta]=1.82-2.13 (m, 4H). 2.44 (m, 2H), 2.69 (m, 1H), 2.79 (m, 2H), 3.70 (m, 2H), 4.01 (s, 3H), 7.53 (m, 2H), 7.85 (m, 2H), 8.24 (s, 1H).

HPLC-MS: 0.963 min, m/z=392.5

### Example P.7

### 1-[[1-(3,4-dichlorophenyl)-3-[(E/Z)-2,2,2-trifluoroethoxyiminomethyl]pyrazol-4-yl]methyl]piperidine-4-carbonitrile (Compound IA-2)

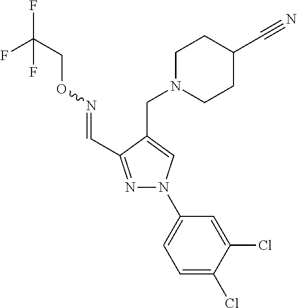 [SMILES: N#CC1CCN(Cc2cn(-c3ccc(Cl)c(Cl)c3)nc2C=NOCC(F)(F)F)CC1]

A solution of 1-[[1-(3,4-dichlorophenyl)-3-formyl-pyrazol-4-yl]methyl]piperidine-4-carbonitrile (obtained in example P.5, 0.34 g, 0.94 mmol, 1.0 equiv.), O-(2,2,2-trifluoroethyl)hydroxylamine hydrochloride (0.15 g, 1.03 mmol, 1.1 equiv.) and sodium acetate (84 mg, 1.03 mmol, 1.1 equiv.) in ethanol (10 mL) was heated at reflux for 3 h. After cooling, water was added and the mixture was extracted with dichloromethane. Combined organic layers were dried over sodium sulfate and concentrated in vacuum. The residue was purified via column chromatography on silica gel to obtain the title compound (0.23 g, 53%).

¹H-NMR (400 MHz, CDCl₃): δ [delta]=1.80-2.05 (m, 4H). 2.41 (m, 2H), 2.68 (m, 1H), 2.79 (m, 2H), 3.69 (m, 2H), 4.52 (q, 2H), 7.55 (m, 2H), 7.87 (m, 2H), 8.36 (s, 1H).

HPLC-MS: 1.042 min, m/z=460.6

By the methods described above for examples P.1 to P-7, the compounds IA-1 to IA-12 and IB-1 to IB-7 of formula (IA) summarized in tables B.1, B.2 and B.3 can be prepared:

### 1) Compounds IA

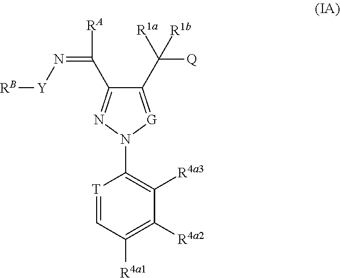

wherein T is CH, Q is Q.1 (4-cyanopiperidin-1-yl), R^{A}, R^{1a} and R^{1b} are H, G is CH and Y is O:

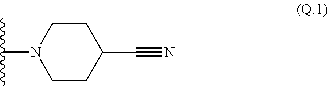 [SMILES: *N1CCC(C#N)CC1]

TABLE B.1

| | | | | | | HPLC- | m/z |
|---|---|---|---|---|---|---|---|
| Compound | | | | | | MS Rₜ | [M + |
| Ex. | R^{B} | R^{4a1} | R^{4a2} | R^{4a3} | Method | (min) | H] |
| IA-1 | CH₃ | Cl | Cl | H | A | 0.963 | 394.5 |
| IA-2 | CH₂CF₃ | Cl | Cl | H | A | 1.042 | 460.6 |
| IA-3 | CH₂CH₃ | Cl | Cl | H | A | 1.028 | 405.8 |
| IA-4 | benzyl | Cl | Cl | H | A | 1.107 | 469.6 |
| IA-5 | CH₃ | CF₃ | H | H | A | 0.954 | 391.9 |
| IA-6 | CH₂CH₃ | CF₃ | H | H | A | 0.999 | 405.9 |
| IA-7 | benzyl | CF₃ | H | H | A | 1.080 | 467.9 |
| IA-8 | CH₂CF₃ | CF₃ | H | H | A | 1.030 | 459.8 |
| IA-9 | H | CF₃ | H | H | A | 0.902 | 377.8 |

2) Compounds of formula IA, wherein T is CH, Q is Q.1 (4-cyanopiperidin-1-yl), R^{1a} and R^{1b} are H, R^{4a3} is H, G is CH and Y is O:

TABLE B.2

| | | | | | | HPLC | | | |
|---|---|---|---|---|---|---|---|---|---|
| Compound | | | | | | Rₜ | | LC-MS | m/z |
| Ex. | R^{A} | R^{B} | R^{4a1} | R^{4a2} | Method | (min) | Method | Rₜ (min) | [M + H] |
| IA-10 | CH₃ | CH₃ | Cl | Cl | B | 5.98 | E | 2.36 | 406 |
| IA-11* | phenyl | CH₃ | Cl | Cl | B | 6.22 | E | 2.47 | 468 |
| IA-12* | phenyl | CH₃ | Cl | Cl | B | 6.28 | E | 2.61 | 468 |

*Compounds IA-11 and IA-12 were obtained as E/Z isomers which could be separated

3) Compounds of formula IA, wherein T is CH, Q is Q.1 (4-cyanopiperidin-1-yl), R^{A}, R^{1a} and R^{1b} are H, G is N and Y is O:

TABLE B.3

| Com- | | | | | | HPLC- | m/z |
|---|---|---|---|---|---|---|---|
| pound | | | | | | MS Rₜ | [M + |
| Ex. | R^{B} | R^{4a1} | R^{4a2} | R^{4a3} | Method | (min) | H] |
| IB-1 | CH₃ | Cl | Cl | H | C | 2.43 | 393.4 |
| IB-2 | CH₂CH₃ | Cl | Cl | H | D | 7.445 | 407.6 |
| IB-3 | CH₃ | CF₃ | H | H | C | 2.38 | 393.5 |
| IB-4 | CH₂CH₃ | CF₃ | H | H | C | 2.54 | 407.5 |
| IB-5 | CH₂CH(CH₃)₂ | Cl | Cl | H | C | 2.98 | 435.4 |
| IB-6 | CH₂CH(CH₃)₂ | CF₃ | H | H | C | 2.88 | 435.4 |
| IB-7 | CH₃ | OCF₃ | H | H | C | 2.49 | 409.5 |

### B. Evaluation of Pesticidal Activity

The activity of the compounds of formula I of the present invention were demonstrated and evaluated by the following biological test.

B.1 Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 25±1° C. and about 75±5% relative humidity for 5 days. Egg and larval mortality was then visually assessed.

In this test, compounds IA-1, IA-2, IA-3, IA-5, IA-6, IA-8, IB-2, IB-3, IB-4 at 2500 ppm showed over 75% mortality in comparison with untreated controls.

### B.2 Orchid Thrips (Dichromothrips Corbetti)

Dichromothrips corbetti adults used for bioassay were obtained from a colony maintained continuously under laboratory conditions. For testing purposes, the test compound is diluted in a 1:1 mixture of acetone:water (vol:vol), plus Kinetic HV at a rate of 0.01% v/v.

Thrips potency of each compound was evaluated by using a floral-immersion technique. All petals of individual, intact orchid flowers were dipped into treatment solution and allowed to dry in Petri dishes. Treated petals were placed into individual resealable plastic along with about 20 adult thrips. All test arenas were held under continuous light and a temperature of about 28° C. for duration of the assay. After 3 days, the numbers of live thrips were counted on each petal. The percent mortality was recorded 72 hours after treatment.

In this test, compounds IA-1, IA-2, IA-3, IA-5, IB-7 at 500 ppm showed over 75% mortality in comparison with untreated controls

### B.3 Cowpea Aphid (Aphis Craccivora)

The active compound is dissolved at the desired concentration in a mixture of 1:1 (vol:vol) distilled water:acetone. Surfactant (Kinetic HV) is added at a rate of 0.01% (vol/vol). The test solution is prepared at the day of use.

Potted cowpea plants were colonized with approximately 50-100 aphids of various stages by manually transferring a leaf tissue cut from infested plant 24 hours before application. Plants were sprayed after the pest population has been recorded. Treated plants are maintained on light carts at about 28° C. Percent mortality was assessed after 72 hours.

In this test, compounds IA-1, IA-2, IA-4, IB-4, IB-7 at 500 ppm showed over 75% mortality in comparison with untreated controls.

B.4 Mediterranean Fruitfly (Ceratitis capitata)

For evaluating control of Mediterranean fruitfly (Ceratitis capitata) the test unit consisted of microtiter plates containing an insect diet and 50-80 C. capitata eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 28±1° C. and about 80±5% relative humidity for 5 days. Egg and larval mortality was then visually assessed.

In this test, compounds IA-1, IA-2, IA-3, IA-5, IA-6, IA-8, IB-1, IB-2 at 2500 ppm showed over 75% mortality in comparison with untreated controls.

B.5 Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28±1° C. and about 80±5% relative humidity for 5 days. Egg and larval mortality was then visually assessed.

In this test, compounds IA-1, IA-2, IA-3, IA-5, IA-6, IA-8, IA-10, IA-11, IA-12, IB-4 at 2500 ppm showed over 75% mortality in comparison with untreated controls.

B.6 Green Peach Aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds IA-1, IA-3, IA-5 at 2500 ppm showed over 75% mortality in comparison with untreated controls.

B.7 Rice Green Leafhopper (Nephotettix virescens)

Rice seedlings were cleaned and washed 24 hours before spraying. The active compounds were formulated in 1:1 acetone:water (vol:vol), and 0.01% vol/vol surfactant (Kinetic HV) was added. Potted rice seedlings were sprayed with 5-6 ml test solution, air dried, covered with Mylar cages cages and inoculated with 10 adults. Treated rice plants were kept at about 28-29° C. and relative humidity of about 50-60%. Percent mortality was recorded after 72 hours.

In this test, compounds IA-1, IA-2, IA-3, IA-5, IB-1, IB-7 at 500 ppm showed over 75% mortality in comparison with untreated controls.

B.8 Diamond Back Moth (Plutella xylostella)

The active compound is dissolved at the desired concentration in a mixture of 1:1 (vol:vol) distilled water:aceteone. Surfactant (Kinetic HV) is added at a rate of 0.01% (vol/vol). The test solution is prepared at the day of use.

Leaves of cabbage were dipped in test solution and air-dried. Treated leaves were placed in petri dishes lined with moist filter paper and inoculated with ten 3^{rd} instar larvae. Mortality was recorded 72 hours after treatment. Feeding damages were also recorded using a scale of 0-100%.

In this test, compounds IA-1, IA-2, IA-3, IA-5, IA-6, IA-8, IB-1, IB-2, IB-4, IB-7 at 500 ppm showed over 75% mortality in comparison with untreated controls.

B.9 Southern Armyworm (Spodoptera eridania), 2nd Instar Larvae

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone:50% water (v/v) into 5 or 10 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, compounds IA-1, IA-2, IA-3, IA-5, IA-6, IA-7, IA-8 IA-10, IB-3, IB-4, IB-6, IB-7 at 300 ppm showed over 75% mortality in comparison with untreated controls.

1-34. (canceled)

35: An azole compound of formula I 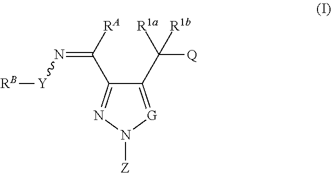
wherein
Q is a radical of formula Q-1, Q-2, Q-3, Q-4 or Q-5 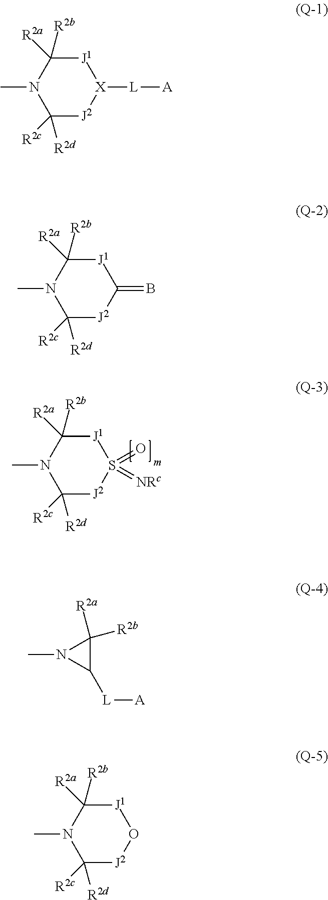
J¹ is a direct bond, —C(R^{3a}R^{3b})—, —C(R^{3a}R^{3b})—C(R^{3a}R^{3b})— or —C(═B)—;
J² is a direct bond, —C(R^{3c}R^{3d})— or —C(═B)—;
X is selected from CR^{3e} and N;
L is a single bond or is selected from the group consisting of C₁-C₁₂-alkylene, C₁-C₁₂-haloalkylene, C₂-C₁₀-alkenylene, C₂-C₁₀-haloalkenylene, C₂-C₁₀-alkynylene, C₂-C₁₀-haloalkynylene, C₃-C₈-cycloalkylene and C₃-C₈-halocycloalkylene, where the 8 aforementioned radicals can be substituted with one or more radicals R¹⁵;
A is selected from the group consisting of hydrogen, halogen, cyano, nitro, —SF₅, —SCN; C₁-C₆-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₆-alkenyl which may be partially or fully halogenated and/or may be substituted by one or more radicals R²⁰; C₂-C₆-alkynyl which may be partially or fully halogenated and/or may be substituted by one or more radicals R²⁰; phenyl which may be substituted by 1, 2, 3, 4 or 5 radicals R²⁴; a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴; —C(═O)R²⁰; —C(═O)OR²¹; —C(═O)N(R²²)R²³; —C(═S)R²⁰; —C(═S)OR²¹;
—C(═S)N(R²²)R²³; —N(R²²)R²³; —NR²²C(═O)R²⁰; —NR²²C(═O)OR²¹; —N═SR³²R³³; —OR²¹; —SR²¹; —S(O)ₚR²¹; —S(O)ₙN(R²²)R²³; —CR^{d}═N—R^{c1}, —C(═N—N(R^{c1})R^{c2})R^{d}, —C(═NR^{c1})N(R^{c2})R^{c3}; —S(═N—R^{c1})R^{a}, —S(═O)(═N—R^{c1})—R^{a}, —C(═O)—N═S(R^{a})₂; —NR^{c1}—S—R^{a}, —NR^{c1}—S(═O)—R^{a},
—NR^{c1}—S(═O)₂—R^{a}; —NR^{c1}—S—N(R^{c2})R^{c3}; —NR^{c1}—S(═O)—N(R^{c2})R^{c3}, —NR^{c1}—S(═O)₂—N(R^{c2})R^{c3}; —N(═C(R^{d})R^{b}), and —N═(C(R^{d})N(R^{c1})R^{c2});
each B is independently selected from O, S, CR^{d1}R^{d2} and NR^{c1};
G is C—R¹⁴ or N;
Z is selected from phenyl which may carry 1, 2, 3, 4 or 5 radicals R^{4a}; a 5- or 6-membered heteroaromatic monocyclic ring and a 8-, 9- or 10-membered heteroaromatic bicyclic ring, the heteroaromatic mono- and bicyclic ring containing 1, 2, 3 or 4 heteroatoms selected from O, N and S as ring members, where the heteroaromatic mono- or bicyclic ring may carry 1, 2 or 3 radicals R^{4a};
Y is O, N—R^{y}, S(O)ₙ or a chemical bond;
R^{A} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₁-C₁₀-alkoxy; C₁-C₁₀-haloalkoxy; C₁-C₁₀-alkylthio; C₁-C₁₀-haloalkylthio; —C(═O)R²⁰; —C(═O)OR²¹; —C(═O)N(R²²)R²³; phenyl which may be substituted by 1, 2, 3, 4 or 5 radicals R²⁴; and a C-bound 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
R^{B} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰;
—N(R²²)R²³; —N(R²²)C(═O)R²⁰; —OR²¹; —SR²¹; —S(O)ₚR²¹; —S(O)ₙN(R²²)R²³; —C(═O)R²⁰; —C(═O)OR²¹; —C(═O)N(R²²)R²³; —C(═S)R²⁰; —C(═S)OR²¹, —C(═S)N(R²²)R²³; —C(═NR²²)R²⁰; phenyl which may be substituted by 1, 2, 3, 4 or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
with the proviso that R^{B} is not —OR²¹ if Y is O;
R^{y} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰;
—N(R²²)R²³; —Si(R²⁶)₂R²⁵; —OR²¹; —SR²¹; —S(O)ₚR²¹; —S(O)ₙN(R²²)R²³; —C(═O)R²⁰; —C(═O)OR²¹; —C(═O)N(R²²)R²³; —C(═S)R²⁰; —C(═S)OR²¹; —C(═S)N(R²²)R²³; —C(═NR²²)R²⁰; phenyl which may be substituted by 1, 2, 3, 4 or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
or R^{B} and R^{y} together form a group ═NR²² or ═NOR²¹;
or R^{B} and R^{y} together form a C₂-C₇ alkylene chain, thus forming, together with the nitrogen atom to which they are bound, a 3-, 4-, 5-, 6-, 7- or 8-membered ring, where the alkylene chain may be interrupted by 1 or 2 O, S and/or NR²⁹ and/or 1 or 2 of the CH₂ groups of the alkylene chain may be replaced by a group C═O, C═S and/or C═NR²⁹; and/or the alkylene chain may be substituted by one or more radicals selected from the group consisting of halogen, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
each R^{a} is independently selected from the group consisting of hydrogen, C₁-C₆-alkyl, C₂-C₆-alkenyl, C₂-C₆-alkynyl, C₃-C₈-cycloalkyl, C₁-C₆-alkylsulfinyl, C₁-C₆-alkylsulfonyl, wherein the aliphatic and cycloaliphatic moieties in the 6 aforementioned radicals may be partially or fully halogenated and/or may carry 1 or 2 substituents selected from C₁-C₄-alkoxy and oxo;
phenyl, benzyl and pyridyl, wherein the three last-mentioned radicals may carry one or more substituents selected from halogen, C₁-C₆-alkyl, C₁-C₆-haloalkyl, C₁-C₆-alkoxy, C₁-C₆-haloalkoxy and C₁-C₆-alkoxycarbonyl;
each R^{b} is independently selected from the group consisting of hydrogen, C₁-C₆-alkyl, C₂-C₆-alkenyl, C₂-C₆-alkynyl, C₃-C₈-cycloalkyl, C₁-C₆-alkoxy, C₁-C₆-alkylthio, C₁-C₆-alkylsulfinyl, C₁-C₆-alkylsulfonyl, wherein the aliphatic and cycloaliphatic moieties in the 8 aforementioned radicals may be partially or fully halogenated and/or may carry 1 or 2 substituents selected from C₁-C₄-alkoxy and oxo;
phenyl, benzyl, pyridyl and phenoxy, wherein the four last-mentioned radicals may carry one or more substituents selected from halogen, C₁-C₆-alkyl, C₁-C₆-haloalkyl, C₁-C₆-alkoxy, C₁-C₆-haloalkoxy, C₁-C₆-alkoxycarbonyl, C₁-C₆-alkylamino and di-(C₁-C₆-alkyl)amino;
R^{c}, R^{c1}, R^{c2} and R^{c3}, independently of each other and independently of each occurrence, are selected from the group consisting of hydrogen, cyano, C₁-C₆-alkyl, C₂-C₆-alkenyl, C₂-C₆-alkynyl, C₃-C₈-cycloalkyl, C₁-C₆-alkoxy, C₁-C₆-alkylthio, C₁-C₆-alkylsulfinyl, C₁-C₆-alkylsulfonyl, wherein the aliphatic and cycloaliphatic moieties in the 8 last-mentioned radicals may be partially or fully halogenated and/or may carry 1 or 2 substituents R¹⁸;
phenyl, benzyl, pyridyl and phenoxy, wherein the four last-mentioned radicals may carry one or more substituents selected from halogen, cyano, C₁-C₆-alkyl, C₁-C₆-haloalkyl, C₂-C₆-alkenyl, C₂-C₆-alkynyl, C₃-C₈-cycloalkyl, C₁-C₆-alkoxy, C₁-C₆-haloalkoxy, C₁-C₆-alkoxycarbonyl, C₁-C₆-alkylamino and di-(C₁-C₆-alkyl)amino; or
R^{c1} and R^{c2}, or R^{c2} and R^{c3}, together with the nitrogen atom to which they are bound, form a 3-, 4-, 5-, 6- or 7-membered saturated, partially unsaturated or maximally unsaturated heterocyclic ring which may additionally contain 1 or 2 further heteroatoms or heteroatom groups selected from N, O, S, NO, SO and SO₂ as ring members, where the heterocyclic ring may be substituted with one or more substituents selected from halogen, C₁-C₄-haloalkyl, C₁-C₄-alkoxy and C₁-C₄-haloalkoxy;
R^{d}, R^{d1} and R^{d2}, independently of each other and independently of each occurrence, are selected from the group consisting of hydrogen, halogen, cyano, nitro, —OH, —SH, —SCN, C₁-C₆-alkyl, C₂-C₆-alkenyl, C₂-C₆-alkynyl, C₃-C₅-cycloalkyl, C₁-C₆-alkoxy, C₁-C₆-alkylthio, C₁-C₆-alkylsulfinyl, C₁-C₆-alkylsulfonyl, wherein the aliphatic and cycloaliphatic moieties in the 8 last-mentioned radicals may be partially or fully halogenated and/or may carry 1 or 2 substituents selected from C₁-C₄-alkoxy and oxo;
—OR^{a}, —NR^{c1}R^{c2}, —S(O)ₙR^{a}, —S(O)ₙNR^{c1}R^{c2}, —C(═O)R^{b}, —C(═O)NR^{c1}R^{c2}, —C(═O)OR^{a}, —C(═S)R^{b}, —C(═S)NR^{c1}R^{c2}, —C(═S)OR^{a}, —C(═S)SR^{a}, —C(═NR^{c1})R^{b}, —C(═NR^{c1})NR^{c2}R^{c3}, phenyl, benzyl, pyridyl and phenoxy, wherein the four last-mentioned radicals may carry one or more substituents selected from halogen, C₁-C₆-alkyl, C₁-C₆-haloalkyl, C₁-C₆-alkoxy and C₁-C₆-haloalkoxy;
R^{1a} is selected from hydrogen, cyano, C₁-C₆-alkyl, C₁-C₆-haloalkyl, C₃-C₆-cycloalkyl, C₃-C₆-halocycloalkyl, carboxyl, —C(═O)R^{5a}, —C(═O)OR^{6a} and —C(═O)NR^{7a}R^{8a};
R^{1b} is selected from hydrogen and C₁-C₆-alkyl;
R^{2a} and R^{2c}, independently of each other and independently of each occurrence, are selected from the group consisting of hydrogen, halogen, cyano, —C(═O)R⁵, —C(═S)R⁵, —C(═O)OR⁶, —C(═O)NR⁷R⁸; —C(═S)NR⁷R⁸, S(O)ₙR¹¹, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷;
R^{2b} and R^{2d}, independently of each other and independently of each occurrence, are selected from the group consisting of hydrogen, halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷; phenyl which may carry 1, 2 or 3 substituents R⁴; and a 5- or 6-membered heteroaromatic ring containing 1, 2 or 3 heteroatoms selected from O, N and S as ring members, where the heteroaromatic ring may carry 1, 2 or 3 substituents R⁴;
R^{3a} and R^{3c}, independently of each other and independently of each occurrence, are selected from the group consisting of hydrogen, halogen, cyano, —C(═O)R⁵, —C(═S)R⁵, —C(═O)OR⁶, —C(═O)NR⁷R⁸; —C(═S)NR⁷R⁸, OR¹², S(O)ₙR¹¹, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷;
R^{3b}, R^{3d} and R^{3e}, independently of each other and independently of each occurrence, are selected from the group consisting of hydrogen, halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, OR¹², S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl, C₃-C₈-cycloalkenyl, wherein the aliphatic and cycloaliphatic moieties in the 6 last-mentioned radicals may be partially or fully halogenated and/or may carry 1 or 2 substituents R¹⁷; phenyl which may carry 1, 2 or 3 substituents R⁴; and a 5- or 6-membered heteroaromatic ring containing 1, 2 or 3 heteroatoms selected from O, N and S as ring members, where the heteroaromatic ring may carry 1, 2 or 3 substituents R⁴;
or R^{2a} and R^{2b}, or R^{2a} and R^{2c}, or R^{2c} and R^{2d}, or R^{3a} and R^{3b}, or R^{3a} and R^{3c}, or R^{3c} and R^{3d}, or R^{2a} and R^{3a}, or R^{2a} and R^{3c}, or R^{2c} and R^{3c}, or R^{2a} and R^{3e}, or R^{2c} and R^{3e} or R^{3a} and R^{3e}, or R^{3c} and R^{3e}, together with the carbon atoms they are bound to, may form a 3- 4-, 5- or 6-membered saturated, partially unsaturated or maximally unsaturated carbocyclic or heterocyclic ring containing 1, 2 or 3 heteroatoms or heteroatom groups selected from N, O, S, NO, SO, SO₂, C(O) and C(S) as ring members, where the carbocyclic or heterocyclic ring may carry 1, 2 or 3 substituents selected from halogen, cyano, C₁-C₄-alkyl, C₁-C₄-haloalkyl, C₁-C₄-alkoxy and C₁-C₄-haloalkoxy;
R⁴ and R^{4a}, independently of each other and independently of each occurrence, are selected from the group consisting of halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, OR¹², S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
or two radicals R⁴ or two radical R^{4a}, when bound to adjacent carbon atoms, together with these carbon atoms may form a 3-, 4-, 5- or 6-membered saturated, partially unsaturated or maximally unsaturated carbocyclic or heterocyclic ring containing 1, 2 or 3 heteroatoms or heteroatom groups selected from N, O, S, NO, SO, SO₂, C(O) and C(S) as ring members, where the carbocyclic or heterocyclic ring may carry 1, 2 or 3 substituents selected from halogen, cyano, C₁-C₄-alkyl, C₁-C₄-haloalkyl, C₁-C₄-alkoxy and C₁-C₄-haloalkoxy;
each R⁵ is independently selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{5a} is independently selected from the group consisting of hydrogen, C₁-C₆-alkyl, C₁-C₆-haloalkyl, C₃-C₈-cycloalkyl and C₃-C₈-halocycloalkyl;
each R⁶ is independently selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{6a} is independently selected from the group consisting of hydrogen, C₁-C₆-alkyl and C₁-C₆-haloalkyl;
R⁷ and R⁸, independently of each other and independently of each occurrence, are selected from the group consisting of hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
or R⁷ and R⁸, together with the nitrogen atom they are bound to, form a 5-, 6- or 7-membered saturated, partially unsaturated or maximally unsaturated heterocyclic ring, where the heterocyclic ring may additionally contain 1 or 2 heteroatoms or heteroatom groups selected from O, N, S, NO, SO, SO₂, C(O) and C(S) as ring members; where the heterocyclic ring may carry 1, 2 or 3 substituents R¹³;
R^{7a} and R^{8a}, independently of each other and independently of each occurrence, are selected from the group consisting of hydrogen, C₁-C₆-alkyl and C₁-C₆-haloalkyl;
R⁹ and R¹⁰, independently of each other and independently of each occurrence, are selected from the group consisting of hydrogen, —C(═O)R⁵, —C(═S)R⁵, —C(═O)OR⁶, —C(═O)NR⁷R⁸; —C(═S)NR⁷R⁸, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
or R⁹ and R¹⁰, together with the nitrogen atom they are bound to, form a 5-, 6- or 7-membered saturated, partially unsaturated or maximally unsaturated heterocyclic ring, where the heterocyclic ring may additionally contain 1 or 2 heteroatoms or heteroatom groups selected from O, N, S, NO, SO, SO₂, C(O) and C(S) as ring members; where the heterocyclic ring may carry 1, 2 or 3 substituents R¹³;
R^{9a} and R^{10a}, independently of each other and independently of each occurrence, are selected from the group consisting of hydrogen, —C(═O)R^{5a}, —C(═S)R^{5a}, —C(═O)OR^{6a}, —C(═O)NR^{7a}R^{8a}; —C(═S)NR^{7a}R^{8a}, C₁-C₆-alkyl and C₁-C₆-haloalkyl;
each R¹¹ is independently selected from C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{11a} is independently selected from C₁-C₆-alkyl and C₁-C₆-haloalkyl;
each R¹² is independently selected from hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷; phenyl which may carry 1, 2 or 3 substituents R¹³; and a 5- or 6-membered heteroaromatic ring containing 1, 2 or 3 heteroatoms selected from O, N and S as ring members, where the heteroaromatic ring may carry 1, 2 or 3 substituents R¹³;
each R^{12a} is independently selected from hydrogen, C₁-C₆-alkyl and C₁-C₆-haloalkyl;
each R¹³ is independently selected from the group consisting of halogen, cyano, nitro, —C(═O)R^{5a}, —C(═S)R^{5a}, —C(═O)OR^{6a}, —C(═O)NR^{7a}R^{8a}; —C(═S)NR^{7a}R^{8a}, NR^{9a}R^{10a}, OR^{12a}, S(O)ₙR^{11a}, S(O)₂NR^{9a}R^{10a}, C₁-C₆-alkyl, C₂-C₆-alkenyl, C₂-C₆-alkynyl, C₃-C₈-cycloalkyl and C₃-C₈-cycloalkyl-C₁-C₄-alkyl, wherein the aliphatic and cycloaliphatic moieties in the 5 last-mentioned radicals may be partially or fully halogenated and/or may carry 1 or 2 substituents R¹⁷;
or two radicals R¹³, when bound to adjacent carbon atoms, together with these carbon atoms may form a 3- 4-, 5- or 6-membered saturated, partially unsaturated or maximally unsaturated carbocyclic or heterocyclic ring containing 1, 2 or 3 heteroatoms or heteroatom groups selected from N, O, S, NO, SO, SO₂, C(O) and C(S) as ring members, where the carbocyclic or heterocyclic ring may carry 1, 2 or 3 substituents selected from halogen, cyano, C₁-C₄-alkyl, C₁-C₄-haloalkyl, C₁-C₄-alkoxy and C₁-C₄-haloalkoxy;
R¹⁴ is selected from the group consisting of hydrogen, halogen, cyano, nitro, —C(═O)R⁵, —C(═S)R⁵, —C(═O)OR⁶, —C(═O)NR⁷R⁸; —C(═S)NR⁷R⁸, NR⁹R¹⁰, OR¹², S(O)ₙR¹¹, S(O)₂NR⁹R¹⁰, C₁-C₆-alkyl, C₂-C₆-alkenyl, C₂-C₆-alkynyl, C₃-C₈-cycloalkyl, C₃-C₈-cycloalkyl-C₁-C₄-alkyl and C₃-C₈-cycloalkenyl, wherein the aliphatic and cycloaliphatic moieties in the 6 last-mentioned radicals may be partially or fully halogenated and/or may carry 1 or 2 substituents R¹⁷;
each R¹⁵ is independently selected from cyano, nitro, —OH, —SH, —SCN, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-dialkylamino, C₁-C₆-alkylaminocarbonyl, C₁-C₆-dialkylaminocarbonyl, phenyl which may carry 1, 2, 3, 4 or 5 radicals R¹⁶; and a 3-, 4-, 5-, 6-, 7- or 8-membered saturated, partially unsaturated or maximally unsaturated heterocyclic ring containing 1, 2, 3 or 4 heteroatoms or heteroatom groups selected from N, S, O, NO, SO and SO₂ and optionally also 1 or 2 groups C(═O) or C(═S) as ring members, where the heterocyclic ring may be substituted by one or more radicals R¹⁶; and
as a substituent on a cycloalkylene or halocycloalkylene moiety, R¹⁵ is additionally selected from C₁-C₆-alkyl, C₁-C₆-haloalkyl, C₂-C₆-alkenyl, C₂-C₆-haloalkenyl, C₂-C₆-alkynyl and C₂-C₆-haloalkynyl;
each R¹⁶ is independently selected from halogen, nitro, C₁-C₄-alkyl, C₁-C₄-haloalkyl, C₁-C₄-alkoxy, C₁-C₄-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-dialkylamino, C₁-C₆-alkylaminocarbonyl and C₁-C₆-dialkylaminocarbonyl;
each R¹⁷ is independently selected from cyano, nitro, OR^{12a} and S(O)ₙR^{11a}; and as a substituent on a cycloalkyl, cycloalkyl-alkyl or cycloalkenyl moiety, R¹⁷ is additionally selected from C₁-C₄-alkyl and C₁-C₄-haloalkyl;
each R¹⁸ is independently selected from cyano, nitro, OR^{12a}, oxo, S(O)ₙR^{11a}, C₃-C₈-cycloalkyl, C₃-C₈-halocycloalkyl, where the cycloaliphatic moieties in the two last-mentioned radicals may be substituted by one or more radicals selected oxo and cyano; C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, C₁-C₆-dialkylamino, C₁-C₆-alkylaminocarbonyl, C₁-C₆-dialkylaminocarbonyl, phenyl which may carry 1, 2, 3, 4 or 5 radicals R¹⁶; and a 3-, 4-, 5-, 6-, 7- or 8-membered saturated, partially unsaturated or maximally unsaturated heterocyclic ring containing 1, 2, 3 or 4 heteroatoms or heteroatom groups selected from N, S, O, NO, SO and SO₂ and optionally also 1 or 2 groups C(═O) or C(═S) as ring members, where the heterocyclic ring may be substituted by one or more radicals R¹⁶;
and as a substituent on a cycloalkyl moiety, R¹⁸ is additionally selected from C₁-C₄-alkyl and C₁-C₄-haloalkyl;
each R²⁰ is independently selected from the group consisting of cyano, azido, nitro, —SCN, SF₅, C₃-C₈-cycloalkyl, C₃-C₈-halocycloalkyl, —Si(R²⁶)₂R²⁵, —OR²¹, —OSO₂R²¹, —SR²¹, —S(O)ₚR²¹, —S(O)ₙN(R²²)R²³, —N(R²²)R²³, —C(═O)N(R²²)R²³, —C(═S)N(R²²)R²³, —C(═O)OR²¹, —C(═O)R³⁰, phenyl which may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
and, in case R²⁰ is bound to a cycloalkyl group or to a heterocyclic ring formed by R^{A} and R^{B} together with the atoms to which they are bound, R²⁰ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl in which the phenyl moiety may be substituted by 1, 2, 3, 4 or 5 radicals R²⁴;
and in groups —C(═O)R²⁰, —C(═S)R²⁰, —C(═NR²²)R²⁰ and —N(R²²)C(═O)R²⁰, R²⁰ may additionally be selected from hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, C₂-C₆-haloalkynyl and benzyl in which the phenyl moiety may be substituted by 1, 2, 3, 4 or 5 radicals R²⁴;
or two geminally bound radicals R²⁰ together form a group selected from ═CR²⁷R²⁸, ═S(O)ₘR²¹, ═S(O)ₘN(R²²)R²³, ═NR²², ═NOR²¹ and ═NNR²²;
or two radicals R²⁰, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;
each R²¹ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R²⁶)₂R²⁵, —SR³¹, —S(O)ₚR³¹, —S(O)ₙN(R²²)R²³, —N(R²²)R²³, —N═CR³²R³³, —C(═O)R³⁴, —C(═O)N(R²²)R²³, —C(═S)N(R²²)R²³, —C(═O)OR³⁴, phenyl which may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
with the proviso that R²¹ is not C₁-C₆-alkoxy or C₁-C₆-haloalkoxy if it is bound to an oxygen atom;
R²² and R²³, independently of each other and independently of each occurrence, are selected from the group consisting of hydrogen, cyano, C₁-C₆-alkyl which may be partially or fully halogenated and/or may be substituted by one or more radicals R³⁰, C₁-C₆-alkoxy, C₁-C₆-haloalkoxy, C₁-C₆-alkylthio, C₁-C₆-haloalkylthio, where the alkyl moiety in the four last-mentioned radicals may be substituted by one or more radicals R³⁰, C₃-C₈-cycloalkyl which may be partially or fully halogenated and/or may be substituted by one or more radicals R³⁰, C₃-C₈-cycloalkyl-C₁-C₄-alkyl where the cycloalkyl moiety may be partially or fully halogenated and/or may be substituted by one or more radicals R³⁰, C₂-C₆-alkenyl which may be partially or fully halogenated and/or may be substituted by one or more radicals R³⁰, C₂-C₆-alkynyl which may be partially or fully halogenated and/or may be substituted by one or more radicals R³⁰, —S(O)ₚR³¹, —S(O)ₙN(R³⁵)R³⁶, phenyl which may be substituted by 1, 2, 3, 4 or 5 radicals R²⁴, benzyl in which the phenyl moiety may be substituted by 1, 2, 3, 4 or 5 radicals R²⁴, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴;
or R²² and R²³ together form a group ═CR²⁷R²⁸;
or R²² and R²³, together with the nitrogen atom to which they are bound, may form a 3-, 4-, 5-, 6- or 7-membered saturated, partially unsaturated or maximally unsaturated heterocyclic ring which may additionally containing 1 or 2 further heteroatoms or heteroatom groups selected from N, O, S, NO, SO and SO₂, as ring members, where the heterocyclic ring may be substituted by one or more radicals R²⁴;
each R²⁴ is independently selected from the group consisting of halogen, cyano, azido, nitro, —SCN, SF₅, C₁-C₁₀-alkyl which may be partially or fully halogenated and/or may be substituted by one or more radicals R³⁰, C₃-C₈-cycloalkyl which may be partially or fully halogenated and/or may be substituted by one or more radicals R³⁰, C₂-C₁₀-alkenyl which may be partially or fully halogenated and/or may be substituted by one or more radicals R³⁰, C₂-C₁₀-alkynyl which may be partially or fully halogenated and/or may be substituted by one or more radicals R³⁰, —Si(R²⁶)₂R²⁵, —OR³¹, —OS(O)ₙR³¹,
—SR³¹, —S(O)ₚR³¹, —S(O)ₙN(R³⁵)R³⁶, —N(R³⁵)R³⁶, C(═O)R³⁰, —C(═O)OR³¹, —C(═NR³⁵)R³⁶, —C(═O)N(R³⁵)R³⁶, —C(═S)N(R³⁵)R³⁶, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;
or two radicals R²⁴ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR³⁵—, —CH₂CH═N—, —CH═CH—NR³⁵—, —OCH═N— and —SCH═N—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R²⁵ and R²⁶, independently of each other and independently of each occurrence, are selected from the group consisting of C₁-C₄-alkyl, C₃-C₆-cycloalkyl, C₁-C₄-alkoxy-C₁-C₄-alkyl, phenyl and benzyl;
R²⁷ and R²⁸, independently of each other and independently of each occurrence, are selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, —C(═O)R³⁰, —C(═O)OR³¹, —C(═NR³⁵)R³⁶, —C(═O)N(R³⁵)R³⁶, —C(═S)N(R³⁵)R³⁶, phenyl which may be substituted by 1, 2, 3, 4, or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R²⁴;
each R²⁹ is independently defined like R^{y};
each R³⁰ is independently selected from the group consisting of cyano, azido, nitro, —SCN, SF₅, C₃-C₈-cycloalkyl, C₃-C₈-halocycloalkyl, —Si(R²⁶)₂R²⁵, —OR³¹, —OSO₂R³¹, —SR³¹, —S(O)ₚR³¹, —S(O)ₙN(R³⁵)R³⁶, —N(R³⁵)R³⁶, —C(═O)N(R³⁵)R³⁶, —C(═S)N(R³⁵)R³⁶, —C(═O)OR³¹, —C(═O)R³¹, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
and, in case R³⁰ is bound to a cycloalkyl group, R³⁰ may additionally be selected from the group consisting of C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl and C₂-C₆-haloalkynyl; and in groups —C(═O)R³⁰, R³⁰ may additionally be selected from hydrogen, halogen, C₁-C₆-alkyl, C₁-C₆-haloalkyl, C₁-C₆-alkoxy-C₁-C₆-alkyl, C₂-C₆-alkenyl, C₂-C₆-haloalkenyl, C₂-C₆-alkynyl, and C₂-C₆-haloalkynyl;
or two geminally bound radicals R³⁰ together form a group selected from ═CR³⁷R³⁸, ═S(O)ₘR³¹, ═S(O)ₘN(R³⁵)R³⁶, ═NR³⁵, ═NOR³¹ and ═NNR³⁵;
or two radicals R³⁰, together with the carbon atoms to which they are bound, form a 3-, 4-, 5-, 6-, 7- or 8-membered saturated or partially unsaturated carbocyclic or heterocyclic ring, where the heterocyclic ring contains 1, 2 or 3 heteroatoms or heteroatom groups selected from N, O, S, NO, SO and SO₂, as ring members;
each R³¹ is independently selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-cycloalkyl-C₁-C₄-alkyl, C₃-C₈-halocycloalkyl, C₂-C₆-alkenyl, C₂-C₆-haloalkenyl, C₂-C₆-alkynyl, C₂-C₆-haloalkynyl, —Si(R²⁶)₂R²⁵, C₁-C₆-alkylaminosulfonyl, amino, C₁-C₆-alkylamino, di-(C₁-C₆-alkyl)-amino, C₁-C₆-alkylcarbonyl, C₁-C₆-haloalkylcarbonyl, aminocarbonyl, C₁-C₆-alkylaminocarbonyl, di-(C₁-C₆-alkyl)aminocarbonyl, C₁-C₆-alkoxycarbonyl, C₁-C₆-haloalkoxycarbonyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; benzyl in which the phenyl moiety may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
with the proviso that R³¹ is not C₁-C₆-alkoxy or C₁-C₆-haloalkoxy if it is bound to an oxygen atom;
R³² and R³³, independently of each other and independently of each occurrence, are selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R²⁴;
with the proviso that R³² and R³³ are not hydrogen if bound to a sulfur atom (i.e. in —N═SR³²R³³, R³² and R³³ are not hydrogen);
each R³⁴ is independently selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl and benzyl;
R³⁵ and R³⁶, independently of each other and independently of each occurrence, are selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; benzyl in which the phenyl moiety may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
or R³⁵ and R³⁶, together with the nitrogen atom to which they are bound, may form a 3-, 4-, 5-, 6- or 7-membered saturated, partially unsaturated or maximally unsaturated heterocyclic ring which may additionally contain 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy;
R³⁷ and R³⁸, independently of each other and independently of each occurrence, are selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, C₁-C₆-alkoxy, C₁-C₆-haloalkoxy, phenyl which may be substituted by 1, 2, 3, 4, or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, which may be substituted by one or more radicals R²⁴;
m is 0 or 1;
n is 0, 1 or 2; and
p is 1 or 2;
or the N-oxides or the tautomers or the agriculturally acceptable salts thereof.

36: The compound of claim 35, where Q is a radical of formula Q-1.

37: The compound of claim 35, where X is CR^{3e}.

38: The compound of claim 37, where CR^{3e} is selected from hydrogen, halogen and C₁-C₆-alkyl, and is in particular hydrogen.

39: The compound of claim 35, where L is selected from a single bond, C₁-C₁₂-alkylene, C₁-C₁₂-haloalkylene, C₂-C₁₀-alkenylene and C₂-C₁₀-haloalkenylene, and in particular from a single bond, C₁-C₁₂-alkylene and C₁-C₁₂-haloalkylene.

40: The compound of claim 39, where L is a single bond.

41: The compound of claim 35, where A is selected from cyano, C₁-C₆-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₆-alkenyl which may be partially or fully halogenated and/or may be substituted by one or more radicals R²⁰; C₂-C₆-alkynyl which may be partially or fully halogenated and/or may be substituted by one or more radicals R²⁰; phenyl which may be substituted by 1, 2, 3, 4 or 5 radicals R²⁴; a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴; —C(═O)R²⁰; —C(═O)OR²¹; —C(═O)N(R²²)R²³; —C(═S)R²⁰;
—C(═S)OR²¹; —C(═S)N(R²²)R²³; —N(R²²)R²³; —NR²²C(═O)R²⁰; —NR²²C(═O)OR²¹; —OR²¹; —SR²¹; —S(O)ₚR²¹; and —S(O)ₙN(R²²)R²³;
where R²⁰, R²¹, R²², R²³, R²⁴, n and p are as defined in claim 1.

42: The compound of claim 41, where A is selected from cyano, C₁-C₆-alkyl which may be partially or fully halogenated and/or may be substituted by one or more radicals R²⁰; C₃-C₈-cycloalkyl which may be partially or fully halogenated and/or may be substituted by one or more radicals R²⁰; phenyl which may be substituted by 1, 2, 3, 4 or 5 radicals R²⁴; a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴; —C(═O)R²⁰; —C(═O)OR²¹; —C(═O)N(R²²)R²³; —N(R²²)R²³,
—NR²²C(═O)R²⁰; —NR²²C(═O)OR²¹; and —OR²¹.

43: The compound of claim 42, where A is selected from cyano, —NR²²C(═O)R²⁰ and —NR²²C(═O)OR²¹; and where R²⁰ is preferably selected from hydrogen, C₁-C₆-alkyl and C₁-C₆-haloalkyl; and is in particular C₁-C₆-alkyl; R²¹ is in particular C₁-C₆-alkyl and R²² is in particular hydrogen; and where A is in particular cyano.

44: The compound of claim 35, where J¹ is
—C(R^{3a}R^{3b})— or —C(═O)— and J² is —C(R^{3c}R^{3d})— or —C(═O)—, where R^{3a}, R^{3b}, R^{3c} and R^{3d} are independently selected from hydrogen and methyl.

45: The compound of claim 44, where J¹ and J² are CH₂.

46: The compound of claim 35, where R^{A} is selected from the group consisting of hydrogen; cyano; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰; C₁-C₁₀-alkoxy; C₁-C₁₀-haloalkoxy; —C(═O)R²⁰;
—C(═O)OR²¹; —C(═O)N(R²²)R²³; phenyl which may be substituted by 1, 2, 3, 4 or 5 radicals R²⁴; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R²⁴.

47: The compound of claim 46, where R^{A} is selected from the group consisting of hydrogen; cyano; C₁-C₆-alkyl; C₁-C₄-haloalkyl; C₁-C₄-alkoxy; C₁-C₄-haloalkoxy; and —C(═O)R²⁰.

48. The compound of claim 46, where R^{A} is hydrogen, C₁-C₄-alkyl or benzyl, and is in particular hydrogen.

49: The compound of claim 35, where Y is O or NR^{y}, where R^{y} is hydrogen or C₁-C₄-alkyl, and where Y is in particular O.

50: The compound of claim 35, where R^{B} is selected from the group consisting of hydrogen; C₁-C₁₀-alkyl which may be partially or fully halogenated and/or may be substituted by one or more radicals R²⁰; C₃-C₈-cycloalkyl which may be partially or fully halogenated and/or may be substituted by one or more radicals R²⁰; C₂-C₁₀-alkenyl which may be partially or fully halogenated and/or may be substituted by one or more radicals R²⁰; C₂-C₁₀-alkynyl which may be partially or fully halogenated and/or may be substituted by one or more radicals R²⁰;
—C(═O)R²⁰; —C(═O)OR²¹; —C(═O)N(R²²)R²³; —C(═S)R²⁰; —C(═S)OR²¹, —C(═S)N(R²²)R²³; —C(═NR²²)R²⁰; phenyl which may be substituted by 1, 2, 3, 4 or 5 radicals R²⁴; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R²⁴.

51: The compound of claim 50, where R^{B} is selected from the group consisting of hydrogen, C₁-C₆-alkyl which may be partially or fully halogenated and/or may be substituted by one radical R²⁰; —C(═O)R²⁰; —C(═O)OR²¹; —C(═O)N(R²²)R²³; —C(═S)R²⁰; —C(═S)OR²¹, —C(═S)N(R²²)R²³; and phenyl which may be substituted by 1, 2 or 3 radicals R²⁴.

52: The compound of claim 51, where the combination of —Y—R^{B} is selected from
—O—R^{B1}, —NR^{y}—C(═O)R^{20a}, —NR^{y}—C(═O)N(R²²)R²³ and —NR^{y}—C(═S)N(R²²)R²³; where
R^{B1} is selected from hydrogen, C₁-C₆-alkyl, C₁-C₆-haloalkyl and a methyl group substituted by one radical R^{20b};
R^{y} is selected from hydrogen and C₁-C₆-alkyl, and is in particular hydrogen;
R^{20a} is selected from hydrogen, C₁-C₄-alkyl, C₁-C₄-haloalkyl, C₃-C₆-cycloalkyl and phenyl, where the phenyl ring may carry 1, 2 or 3 substituents selected from halogen, CN, C₁-C₄-alkyl, C₁-C₄-haloalkyl, C₁-C₄-alkoxy and C₁-C₄-haloalkoxy;
R^{20b} is selected from cyano, C₃-C₆-cycloalkyl and phenyl, where the phenyl ring may carry 1, 2 or 3 substituents selected from halogen, CN, C₁-C₄-alkyl, C₁-C₄-haloalkyl, C₁-C₄-alkoxy and C₁-C₄-haloalkoxy;
R²² is selected from hydrogen, C₁-C₄-alkyl and C₁-C₄-haloalkyl; and
R²³ is selected from hydrogen, CN, C₁-C₆-alkyl; C₁-C₆-haloalkyl; C₃-C₆-cycloalkyl; C₃-C₆-halocycloalkyl; C₃-C₆-cycloalkyl-C₁-C₄-alkyl; C₃-C₆-halocycloalkyl-C₁-C₄-alkyl; C₁-C₄-alkylcarbonyl, C₁-C₄-haloalkylcarbonyl, C₁-C₄-alkoxycarbonyl, C₁-C₄-haloalkoxycarbonyl, phenyl; benzyl and a 5- or 6-membered saturated, partially unsaturated or maximally unsaturated heterocyclic ring containing 1, 2 or 3 heteroatoms or heteroatom groups selected from N, O, S, NO, SO and SO₂, as ring members, where the phenyl or heterocyclyl rings in the three last-mentioned radicals may carry 1, 2 or 3 substituents selected from halogen, CN, C₁-C₄-alkyl, C₁-C₄-haloalkyl, C₁-C₄-alkoxy and C₁-C₄-haloalkoxy.

53: The compound of claim 52, where
R^{B1} is selected from hydrogen, C₁-C₄-alkyl, C₁-C₄-haloalkyl and benzyl;
R^{y} is selected from hydrogen and C₁-C₄-alkyl, and is in particular hydrogen;
R^{20a} is C₁-C₄-alkyl;
R²² is hydrogen; and
R²³ is selected from hydrogen, C₁-C₆-alkyl and C₁-C₆-haloalkyl.

54: The compound of claim 35, where G is C—R¹⁴, where R¹⁴ is hydrogen.

55: The compound of claim 35, where Z is selected from phenyl which may carry 1, 2 or 3 radicals R^{4a}; and pyridyl which may carry 1, 2 or 3 radicals R^{4a}; and is in particular phenyl which carries 1 or 2 radicals R^{4a}.

56: The compound of claim 55, where each R^{4a} is independently selected from halogen, C₁-C₄-alkyl, C₁-C₄-haloalkyl, C₁-C₄-alkoxy and C₁-C₄-haloalkoxy, and in particular from halogen, trifluoromethyl and trifluoromethoxy.

57: The compound of claim 35, where R^{2a}, R^{2b}, R^{2c} and R^{2d} are hydrogen.

58: The compound of claim 35, where R^{1a} and R^{1b} are hydrogen.

59: The compound according to claim 35 of the formula I-A

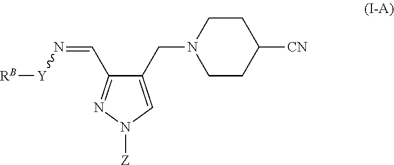

60: The compound of claim 59, where Z is 3,4-dichlorophenyl or 4-(trifluoromethyl)-phenyl; and
Y is O and R^{B} is hydrogen, methyl, ethyl, 2,2,2-trifluoroethyl or benzyl; or
Y is NH and R^{B} is acetyl, —C(═O)NHR²³ or —C(═S)NHR²³, where R²³ is methyl, ethyl or 2,2,2-trifluoroethyl.

61: The compound of claim 59, where Z is 3,4-dichlorophenyl, 4-(trifluoromethyl)-phenyl or 4-(trifluoromethoxy)-phenyl; Y is O; and R^{B} is hydrogen, C₁-C₆-alkyl, 2,2,2-trifluoroethyl or benzyl.

62: An agricultural or veterinary composition comprising at least one compound of the formula I, as defined in claim 35, a stereoisomer thereof and/or at least one agriculturally or veterinarily acceptable salt thereof, and at least one inert liquid and/or solid agriculturally or veterinarily acceptable carrier.

63: A method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of at least one imine compound of the formula I as defined in claim 35, a stereoisomer thereof and/or at least one agriculturally acceptable salt thereof.

64: The method of claim 65, for protecting plants from attack or infestation by invertebrate pests, which method comprises treating the plants with a pesticidally effective amount of at least one compound of the formula I as defined in claim 35, a stereoisomer thereof and/or at least one agriculturally acceptable salt thereof.

65: The method of claim 66, for protecting plant propagation material and/or the plants which grow therefrom from attack or infestation by invertebrate pests, which method comprises treating the plant propagation material with a pesticidally effective amount of at least one compound of the formula I as defined in claim 35, a stereoisomer thereof and/or at least one agriculturally acceptable salt thereof.

66: Plant propagation material, comprising at least one compound of the formula I as defined in claim 35, a stereoisomer thereof and/or at least one agriculturally acceptable salt thereof.
